(19) United States
(12) Patent Application Publication
(10) Pub. No.: US 2022/0252576 A1
(43) Pub. Date: Aug. 11, 2022

(54) Assay

(71) Applicant: The University of Manchester, Manchester, Greater Manchester (GB)

(72) Inventors: Susan Kimber, Manchester, Greater Manchester (GB); Philip Lewis, Manchester, Greater Manchester (GB)

(21) Appl. No.: 17/629,189

(22) PCT Filed: Jul. 22, 2020
(86) PCT No.: PCT/GB2020/051752

§ 371 Date: Jan. 21, 2022

(30) Foreign Application Priority Data
Jul. 22, 2019 (GB) 1910477.7
Oct. 16, 2019 (GB) 1914959.0

(51) Int. Cl. G01N 33/50

(52) U.S. Cl. CPC G01N 33/5073

(57) ABSTRACT

The present invention relates to methods for assessing the pluripotent state of a stem cell culture by determining the presence of marker proteins, related kits and uses.

### INTRODUCTION

Human pluripotent stem cells (hPSCs), have the potential to be an important source of virtually any cell type for basic research, drug development, and clinical cell therapies. Although the clinical potential of regenerative medicine, including approaches using human pluripotent stem cells, remains unrivalled, major hurdles exist in translating promising treatments into scalable, reproducible commercial processes. In particular, maintaining hPSCs in their pluripotent state has proven challenging and labour intensive. Whilst advances have been made in automating the culture and expansion of high quality PSCs, such as by the development of the SelecT (TAP biosystems—Hertfordshire, UK), Freedom EVO (TECAN Trading AG, Switzerland) systems, or technologies developed by Tokyo Electron (Kyoto, Japan); an issue which remains to be overcome in PSC culture of all scales is the absence of a rapid, reproducible, non-invasive and quantitative metric for pluripotent cultures.

The most commonly used techniques all have major drawbacks when it comes to their use in PSC manufacture. For instance, using immunofluorescence for assessing the core pluripotency transcription factors and cell surface markers does not accurately pick up initial loss of pluripotency in entire cultures, but rather the loss of pluripotency in individual cells. Immunofluorescence is also imprecise, non- quantitative, cannot be integrated into the manufacturing process, and the tested cells cannot be reintroduced back into the process. Using flow cytometry for identifying loss of pluripotency markers shows run variability and sacrifices cell product. Currently the most quantifiable metrics of pluripotency available are RNA-based assays on cells e.g. Pluri-test [1] and ScoreCard [2], or teratoma assays [3]. However, these metrics are not currently cost-effective, require sacrifice of cell product, and take too long to provide results on at-risk cultures which have likely been permanently compromised by the time the results are available. The most rapid and cost-effective tool researchers currently have is analysis of cell morphology, however this is inherently subjective. While attempts have been made to automate and quantify the characteristics of PSC morphology [4, 5], it remains a low-resolution way in which to measure pluripotency [6]. There are thus no convenient quantitative assays which can pick up very early loss of pluripotency.

It is an object of the present invention to address one or more problems associated with the current assessment of loss of pluripotency. It would be desirable to identify one or more biomarkers which could be used to identify the loss, immanent loss, or potential loss, of pluripotency. It would be further desirable if the one or more biomarkers were detectable in the culture media so as to minimise the disruption to cell growth and to circumvent the use of any cell lysate in the analysis. Furthermore, it would be desirable to provide a method of assessing or detecting the loss of pluripotency which is quick, reliable and easy to undertake.

### SUMMARY

The invention relates to a method for assessing the pluripotent state of a stem cells culture. The inventors have shown that there are a number of proteins in the secretome of PSCs that are rapidly decreased or increased during dissolution of pluripotency. Thus, several high confidence protein biomarkers were identified which demonstrated consistent correlation with the pluripotent cell state. These protein biomarkers may be utilised as a rapid diagnostic for pluripotency loss.

Additionally, the inventors sought to overcome the absence of a functional normalisation method for secreted protein quantification by developing a novel paired-protein ratio metric. This was based on the observation that proteins which increased or decreased in response to the same stimulus would change more consistently and detectibly in relation to each other, than to other normalisation standards. A number of protein biomarkers in the secretome of PSCs were identified that gave protein-pair ratios that consistently and robustly distinguished the pluripotent from non-pluripotent stem cell cultures, irrespective of the type of cells and culture used.

The assessment of protein pairs to determine the pluripotent state of stem cell cultures provides additional benefits over assessing the abundance of individual proteins in the stem cell culture. Assessment of protein pairs gives a more robust, statistically significant, reliable and rapid quantitative readout of pluripotent stem cell state. This can be used as an early warning of culture quality degradation at a stage when the loss of pluripotency can be recovered without sacrifice of stem cell product. The assessment of the protein biomarkers of the invention, in particular the protein pairs of the invention, also allows intermittent or continuous culture monitoring during the scale up and manufacture of PSCs for cell therapy or for their use in pharmaceutical drug development and toxicity testing.

Thus, in one aspect, the invention relates to a method for determining the pluripotent state of a stem cell culture said method comprising culturing stem cells and

a) measuring the relative abundance of two proteins selected from: Argininosuccinate synthetase 1 (ASS1), Creatine kinase B-type (CKB), chondromodulin-1 (CNMD), Cochlin (COCH), Fibroblast Growth Factor Receptor 1 (FGFR1), Follistatin (FST), Olfactomedin-like 3 (OLFML3), Serpin B1 (SERPINB1), Stomatin (STOM), A disintegrin and metalloproteinase with thrombospondin motifs 8 (ADAMTS8), Amyloid Beta Precursor Protein (APP), UDP-GlcNAc:BetaGal Beta-1,3-N-Acetylglucosaminyltransferase 7 (B3GNT7), Chromogranin A (CHGA), EGF-containing fibulin-like extracellular matrix protein 1 (EFEMP1), Ephrin type-A receptor 1 (EPHA1), Exostosin-like 2 (EXTL2), Fibulin 2 (FBLN2), Fibroblast Growth Factor 2 (FGF2), Plasma alpha-L-fucosidase (FUCA2), Polypeptide N-acetylgalactosaminyltransferase 1 (GALNT), Gamma-Glutamyl Hydrolase (GGH), Beta-hexosaminidase subunit beta (HEXB), Insulin-like growth factor-binding protein 4 (IGFBP4), Nidogen 1 (NID1), Neuronal Pentraxin-2 (NPTX2), Neurotensin (NTS), Protocadherin beta-5 (PCDHB5), Semaphorin 3A (SEMA3A), Semaphorin 3F (SEMA3F), Secreted Frizzled Related Protein 2 (SFRP2), Metalloproteinase inhibitor 4 (TIMP4), Tumor necrosis factor receptor superfamily member 8 (TNFRSF8) and WAP four-disulfide core domain protein 2 (WFDC2) and comparing said relative abundance to a reference value and/or

b) measuring the absolute abundance of one or more protein selected from Argininosuccinate synthetase 1 (ASS1), Creatine kinase B-type (CKB), chondromodulin-1 (CNMD), Cochlin (COCH), Fibroblast Growth Factor Receptor 1 (FGFR1), Follistatin (FST), Olfactomedin-like 3 (OLFML3), Serpin B1 (SERPINB1), Stomatin (STOM), A disintegrin and metalloproteinase with thrombospondin motifs 8 (ADAMTS8), Amyloid Beta Precursor Protein (APP), UDP-GlcNAc:BetaGal Beta-1,3-N-Acetylglucosaminyltransferase 7 (B3GNT7), Chromogranin A (CHGA), EGF-containing fibulin-like extracellular matrix protein 1 (EFEMP1), Ephrin type-A receptor 1 (EPHA1), Exostosin-like 2 (EXTL2), Fibulin 2 (FBLN2), Fibroblast Growth Factor 2 (FGF2), Plasma alpha-L-fucosidase (FUCA2), Polypeptide N-acetylgalactosaminyltransferase 1 (GALNT), Gamma-Glutamyl Hydrolase (GGH), Beta-hexosaminidase subunit beta (HEXB), Insulin-like growth factor-binding protein 4 (IGFBP4), Nidogen 1 (NID1), Neuronal Pentraxin-2 (NPTX2), Neurotensin (NTS), Protocadherin beta-5 (PCDHB5), Semaphorin 3A (SEMA3A), Semaphorin 3F (SEMA3F), Secreted Frizzled Related Protein 2 (SFRP2), Metalloproteinase inhibitor 4 (TIMP4), Tumor necrosis factor receptor superfamily member 8 (TNFRSF8) and WAP four-disulfide core domain protein 2 (WFDC2) and comparing said absolute abundance to a reference value.

The invention also relates to the use of one or more protein biomarker selected from Argininosuccinate synthetase 1 (ASS1), Creatine kinase B-type (CKB), chondromodulin-1 (CNMD), Cochlin (COCH), Fibroblast Growth Factor Receptor 1 (FGFR1), Follistatin (FST), Olfactomedin-like 3 (OLFML3), Serpin B1 (SERPINB1), Stomatin (STOM), A disintegrin and metalloproteinase with thrombospondin motifs 8 (ADAMTS8), Amyloid Beta Precursor Protein (APP), UDP-GlcNAc:BetaGal Beta-1,3-N-Acetylglucosaminyltransferase 7 (B3GNT7), Chromogranin A (CHGA), EGF-containing fibulin-like extracellular matrix protein 1 (EFEMP1), Ephrin type-A receptor 1 (EPHA1), Exostosin-like 2 (EXTL2), Fibulin 2 (FBLN2), Fibroblast Growth Factor 2 (FGF2), Plasma alpha-L-fucosidase (FUCA2), Polypeptide N-acetylgalactosaminyltransferase 1 (GALNT), Gamma-Glutamyl Hydrolase (GGH), Beta-hexosaminidase subunit beta (HEXB), Insulin-like growth factor-binding protein 4 (IGFBP4), Nidogen 1 (NID1), Neuronal Pentraxin-2 (NPTX2), Neurotensin (NTS), Protocadherin beta-5 (PCDHB5), Semaphorin 3A (SEMA3A), Semaphorin 3F (SEMA3F), Secreted Frizzled Related Protein 2 (SFRP2), Metalloproteinase inhibitor 4 (TIMP4), Tumor necrosis factor receptor superfamily member 8 (TNFRSF8) and WAP four-disulfide core domain protein 2 (WFDC2) in assessing the pluripotent state of a stem cell culture. For example, a pair as exemplified herein is used.

The invention also relates to a kit comprising:

a) one or more components which can bind to or otherwise detect one or more of the following proteins: ASS1, CKB, CNMD, COCH, FGFR1, FST, OLFML3, SERPINB1, STOM, ADAMTS8, APP, B3GNT7, CHGA, EFEMP1, EPHA1, EXTL2, FBLN2, FGF2, FUCA2, GALNT, GGH, HEXB, IGFBP4, NID1, NPTX2, NTS, PCDHB5, SEMA3A, SEMA3F, SFRP2, TIMP4, TNFRSF8 and/or WFDC2; and optionally

b) means for quantifying binding and/or detection of the one or more components with the one or more proteins, such as an antibody or antibody fragment.

### Figures

The invention is further described in the following non-limiting figures:

FIG. 1. Experimental design for sample collection and data processing. A) Cells were cultured in E8 for at least two passages prior to the onset of the experiment. Cells were then plated in E8 medium containing Rock inhibitor onto Vitronectin-N coated flasks and were allowed to settle for 24 hours before onset of experimental conditions. Cells were washed in PBS, then half were cultured in E8, and half in E6, with full media replacement after 24 hours. After 48 hours of culture under experimental conditions the media was collected, spun, concentrated and processed for LC-MS/MS. The cells were collected and processed for quality control, and RNA-Seq analysis. These data were then analysed by multiple-integrated methods before a subset of proteins were chosen for further, targeted analyses. B) Immunocytochemistry of Man-13 cells cultured in E8 (control) or after 48 hours of culture in E6. C) Flow cytometry of Rebl.PAT and Man-13 cells cultured in E8, or after 48 hours of culture in E6. For Rebl.PAT cells, extra flasks were cultured alongside experimental flasks to allow for observation of cell state at the 24-hour time-point (shown for Rebl.PAT).

FIG. 2. Proteomics summary. A) Summary of the proteomics experimental inputs and outputs. B) Pie charts demonstrating the proportion of proteins which are identified as secreted by SignalP 4.0 (Classical) or SecretomeP 2.0 (Non-classical) or by their Uniprot peptide sequence. Extracellular Gene Ontology (GO) terms used for classification of proteins were extracellular matrix, extracellular space, extracellular vesicle, cell surface and extracellular region. All proteins used for this analysis had one or more unique peptides, and a confidence score of 30 or more in all three proteomics runs. C) GoSlim Cellular Component summary from WebGestalt of the popular GO terms for each of the categories described in C. Enrichment to p<0.05. Extracellular GO terms are highlighted with black bars.

FIG. 3. Correlation between inter-condition log fold-changes between mRNA and protein data. The E6/E8 inter-condition log-fold change was compared across secretomics and corresponding RNA-Seq data where an Entrez ID was readily available for the proteins identified by proteomics. These data show that there is good agreement in the direction of change between proteomics data and the corresponding RNA-seq data. Only IDs which were q<0.05 significant by secretomics and RNA-Seq data were used for this comparison to protect against stochastic changes with little biological relevance. The same RNA-Seq dataset was used for comparison against both Man-13 proteomics runs.

FIG. 4. Protein-pair ratio methodology. A) Secreted proteomics is highly vulnerable to changes in total protein abundance. On top of biological and technical variance, the relative contributions of different protein sources demonstrated in (A) can affect the normalisation of proteins of interest, increasing statistical noise. Whilst normalised individual abundances may vary, the relative abundance of proteins responding robustly to the same stimulus of interest should remain relatively consistent between those conditions. For example, the two marker proteins presented in B), show a significant change E8 and E6 in each Mass spectrometry run (q<0.05), however the individual abundance of these proteins changes drastically between runs. Box plot whiskers represent minimum and maximum values with the exception of outliers (shown as dots). C) by investigating the relationship between these proteins, it becomes apparent that across all runs, with the exception of outliers, in all instances NPTX2 individual abundance is less than 3× that of OLFML3 in E6, however it is over 9× the individual abundance of OLFML3 in E8. D) By calculating the relative abundances of these proteins in every sample, the differences between conditions by this metric are larger than when comparing individual abundances, and more consistent between MS/MS runs.

FIG. 5. Identification of robust marker proteins through paired-protein ratio analysis. A) Ratios of the relative abundances of proteins were calculated by compared every against another protein. These ratios were tested for FDR adjusted statistical significance between E8 and E6. Protein-pair combinations demonstrating q<0.05 across Man-13 run 1 (M13 R1), Man-13 Run 2 (M13 R2) and Rebl.PAT, were used to create a network; proteins acting as nodes, and significant changes between the relative abundances of those proteins between E8 and E6 as edges between pairs of proteins. B) Proteins which demonstrated the most consistent changes against other proteins were isolated by identifying the 30 most interconnected proteins. C) Data summary for 8 proteins selected for antibody assays based upon both their statistical and biological properties.

FIG. 6. MS/MS individual abundance and relative abundance box plots for the marker proteins chosen for confirmation via Western blot. Individual abundances (left column and top row) and relative abundance protein pairs (intersection of the corresponding row/column) shown are of the marker proteins selected for confirmation by Western blot based upon their paired relative abundances. Box plot y axes either show Log2-transformed individual abundance (for individual markers) or relative abundance expressed as the Log2-transformed E6 marker individual abundance subtracted from the Log2-transformed E8 marker individual abundance. Box plot whiskers represent minimum and maximum values with the exception of outliers (shown as dots).

FIG. 7. A) Re-establishment of marker relative abundances after a 7-day rescue. Man-1 cells were cultured as described in (1.A), but after media collection at 48 hours, E6 cultured cells were passaged and returned to E8 for 7 days. Each protein pair was tested on a single membrane. Representative Western blot bands of spent media from the rescue experiment with corresponding densitometry measurements for (B) OLFML3/NID1 and (C) FST/NPTX2. Densitometry was calculated in ImageJ. Membranes were imaged using the LICOR Odyssey system.

FIG. 8. A) Western blots of marker-proteins in conditioned media. Equal quantities of concentrated conditioned media were run for each condition, and the same membrane probed with both antibodies for each pair. Membranes were imaged using the LICOR Odyssey system. Quantification for these blots is show in in FIG. 7A. B) Paraffin sections (7 μm) of teratomas generated by subcutaneous implantation of human pluripotent stem cells cultured in in E8 (Man13) or after 48 h in E6 (Man13 or ReblPat), followed by 7 days recovery in E8; stained for morphological and protein markers of the 3 germ layers. Paraffin section were stained with Haematoxalin and eosin or with antibodies to early endoderm (GATA6) neurectoderm (Beta3 tubulin) or smooth muscle actin in mesoderm (alpha SMA). Scale bar [=⅙th width=]100 um.

FIG. 9. A) ADAMTS8/E6 Marker Proteins—Relative abundances. Box plots showing the relative abundances calculated by dividing the ADAMTS8 individual abundance by the individual abundances of each of the selected E6 marker proteins. Box plot whiskers represent minimum and maximum values with the exception of outliers (shown as dots). B) Table showing the relative abundances of each protein-pair in each condition and cell line and the corresponding inter-condition fold-change and T-test for the significance of the difference between conditions.

FIG. 10. A) APP/E6 Marker Proteins—Relative abundances. Box plots showing the relative abundances calculated by dividing APP individual abundances by the individual abundances of each of the selected E6 marker proteins. Box plot whiskers represent minimum and maximum values with the exception of outliers (shown as dots). B) Table showing the relative abundances of each protein-pair in each condition and cell line and the corresponding inter-condition fold-change and T-test for the significance of the difference between conditions.

FIG. 11. A) B3GNT7/E6 Marker Proteins—Relative abundances. Box plots showing the relative abundances calculated by dividing B3GNT7 individual abundances by the individual abundances of each of the selected E6 marker proteins. Box plot whiskers represent minimum and maximum values with the exception of outliers (shown as dots). B) Table showing the relative abundances of each protein-pair in each condition and cell line and the corresponding inter-condition fold-change and T-test for the significance of the difference between conditions.

FIG. 12. A) CHGA/E6 Marker Proteins—Relative abundances. Box plots showing the relative abundances calculated by dividing CHGA individual abundances by the individual abundances of each of the selected E6 marker proteins. Box plot whiskers represent minimum and maximum values with the exception of outliers (shown as dots). B) Table showing the relative abundances of each protein-pair in each condition and cell line and the corresponding inter-condition fold-change and T-test for the significance of the difference between conditions.

FIG. 13. A) EFEMP1/E6 Marker Proteins—Relative abundances. Box plots showing the relative abundances calculated by dividing EFEMP1 individual abundances by the individual abundances of each of the selected E6 marker proteins. Box plot whiskers represent minimum and maximum values with the exception of outliers (shown as dots). B) Table showing the relative abundances of each protein-pair in each condition and cell line and the corresponding inter-condition fold-change and T-test for the significance of the difference between conditions.

FIG. 14. A) EPHA1/E6 Marker Proteins—Relative abundances. Box plots showing the relative abundances calculated by dividing EPHA1 individual abundances by the individual abundances of each of the selected E6 marker proteins. Box plot whiskers represent minimum and maximum values with the exception of outliers (shown as dots). B) Table showing the relative abundances of each protein-pair in each condition and cell line and the corresponding inter-condition fold-change and T-test for the significance of the difference between conditions.

FIG. 15. A) EXTL2/E6 Marker Proteins—Relative abundances. Box plots showing the relative abundances calculated by dividing EXTL2 individual abundances by the individual abundances of each of the selected E6 marker proteins. Box plot whiskers represent minimum and maximum values with the exception of outliers (shown as dots). B) Table showing the relative abundances of each protein-pair in each condition and cell line and the corresponding inter-condition fold-change and T-test for the significance of the difference between conditions.

FIG. 16. A) FBLN2/E6 Marker Proteins—Relative abundances. Box plots showing the relative abundances calculated by dividing FBLN2 individual abundances by the individual abundances of each of the selected E6 marker proteins. Box plot whiskers represent minimum and maximum values with the exception of outliers (shown as dots). B) Table showing the relative abundances of each protein-pair in each condition and cell line and the corresponding inter-condition fold-change and T-test for the significance of the difference between conditions.

FIG. 17. A) FGF2/E6 Marker Proteins—Relative abundances. Box plots showing the relative abundances calculated by dividing FGF2 individual abundances by the individual abundances of each of the selected E6 marker proteins. Box plot whiskers represent minimum and maximum values with the exception of outliers (shown as dots). B) Table showing the relative abundances of each protein-pair in each condition and cell line and the corresponding inter-condition fold-change and T-test for the significance of the difference between conditions.

FIG. 18. A) FUCA2/E6 Marker Proteins—Relative abundances. Box plots showing the relative abundances calculated by dividing FUCA2 individual abundances by the individual abundances of each of the selected E6 marker proteins. Box plot whiskers represent minimum and maximum values with the exception of outliers (shown as dots). B) Table showing the relative abundances of each protein-pair in each condition and cell line and the corresponding inter-condition fold-change and T-test for the significance of the difference between conditions.

FIG. 19. A) GALNT1/E6 Marker Proteins—Relative abundances. Box plots showing the relative abundances calculated by dividing GALNT1 individual abundances by the individual abundances of each of the selected E6 marker proteins. Box plot whiskers represent minimum and maximum values with the exception of outliers (shown as dots). B) Table showing the relative abundances of each protein-pair in each condition and cell line and the corresponding inter-condition fold-change and T-test for the significance of the difference between conditions.

FIG. 20. A) GGH/E6 Marker Proteins—Relative abundances. Box plots showing the relative abundances calculated by dividing GGH individual abundances by the individual abundances of each of the selected E6 marker proteins. Box plot whiskers represent minimum and maximum values with the exception of outliers (shown as dots). B) Table showing the relative abundances of each protein-pair in each condition and cell line and the corresponding inter-condition fold-change and T-test for the significance of the difference between conditions.

FIG. 21. A) HEXB/E6 Marker Proteins—Relative abundances. Box plots showing the relative abundances calculated by dividing HEXB individual abundances by the individual abundances of each of the selected E6 marker proteins. Box plot whiskers represent minimum and maximum values with the exception of outliers (shown as dots). B) Table showing the relative abundances of each protein-pair in each condition and cell line and the corresponding inter-condition fold-change and T-test for the significance of the difference between conditions.

FIG. 22. A) IGFBP4/E6 Marker Proteins—Relative abundances. Box plots showing the relative abundances calculated by dividing IGFBP4 individual abundances by the individual abundances of each of the selected E6 marker proteins. Box plot whiskers represent minimum and maximum values with the exception of outliers (shown as dots). B) Table showing the relative abundances of each protein-pair in each condition and cell line and the corresponding inter-condition fold-change and T-test for the significance of the difference between conditions.

FIG. 23. A) NID1/E6 Marker Proteins—Relative abundances. Box plots showing the relative abundances calculated by dividing NID1 individual abundances by the individual abundances of each of the selected E6 marker proteins. Box plot whiskers represent minimum and maximum values with the exception of outliers (shown as dots). B) Table showing the relative abundances of each protein-pair in each condition and cell line and the corresponding inter-condition fold-change and T-test for the significance of the difference between conditions.

FIG. 24. A) NPTX2/E6 Marker Proteins—Relative abundances. Box plots showing the relative abundances calculated by dividing NPTX2 individual abundances by the individual abundances of each of the selected E6 marker proteins. Box plot whiskers represent minimum and maximum values with the exception of outliers (shown as dots). B) Table showing the relative abundances of each protein-pair in each condition and cell line and the corresponding inter-condition fold-change and T-test for the significance of the difference between conditions.

FIG. 25. A) NTS/E6 Marker Proteins—Relative abundances. Box plots showing the relative abundances calculated by dividing NTS individual abundances by the individual abundances of each of the selected E6 marker proteins. Box plot whiskers represent minimum and maximum values with the exception of outliers (shown as dots). B) Table showing the relative abundances of each protein-pair in each condition and cell line and the corresponding inter-condition fold-change and T-test for the significance of the difference between conditions.

FIG. 26. A) PCDHBS/E6 Marker Proteins—Relative abundances. Box plots showing the relative abundances calculated by dividing PCDHBS individual abundances by the individual abundances of each of the selected E6 marker proteins. Box plot whiskers represent minimum and maximum values with the exception of outliers (shown as dots). B) Table showing the relative abundances of each protein-pair in each condition and cell line and the corresponding inter-condition fold-change and T-test for the significance of the difference between conditions.

FIG. 27. A) SEMA3A/E6 Marker Proteins—Relative abundances. Box plots showing the relative abundances calculated by dividing SEMA3A individual abundances by the individual abundances of each of the selected E6 marker proteins. Box plot whiskers represent minimum and maximum values with the exception of outliers (shown as dots). B) Table showing the relative abundances of each protein-pair in each condition and cell line and the corresponding inter-condition fold-change and T-test for the significance of the difference between conditions.

FIG. 28. A) SEMA3F/E6 Marker Proteins—Relative abundances. Box plots showing the relative abundances calculated by dividing SEMA3F individual abundances by the individual abundances of each of the selected E6 marker proteins. Box plot whiskers represent minimum and maximum values with the exception of outliers (shown as dots). B) Table showing the relative abundances of each protein-pair in each condition and cell line and the corresponding inter-condition fold-change and T-test for the significance of the difference between conditions.

FIG. 29. A) SFRP2/E6 Marker Proteins—Relative abundances. Box plots showing the relative abundances calculated by dividing SFRP2 individual abundances by the individual abundances of each of the selected E6 marker proteins. Box plot whiskers represent minimum and maximum values with the exception of outliers (shown as dots). B) Table showing the relative abundances of each protein-pair in each condition and cell line and the corresponding inter-condition fold-change and T-test for the significance of the difference between conditions.

FIG. 30. A) TIMP4/E6 Marker Proteins—Relative abundances. Box plots showing the relative abundances calculated by dividing TIMP4 individual abundances by the individual abundances of each of the selected E6 marker proteins. Box plot whiskers represent minimum and maximum values with the exception of outliers (shown as dots). B) Table showing the relative abundances of each protein-pair in each condition and cell line and the corresponding inter-condition fold-change and T-test for the significance of the difference between conditions.

FIG. 31. A) TNFRSF8/E6 Marker Proteins—Relative abundances. Box plots showing the relative abundances calculated by dividing TNFRSF8 individual abundances by the individual abundances of each of the selected E6 marker proteins. Box plot whiskers represent minimum and maximum values with the exception of outliers (shown as dots). B) Table showing the relative abundances of each protein-pair in each condition and cell line and the corresponding inter-condition fold-change and T-test for the significance of the difference between conditions.

FIG. 32. A) WFDC2/E6 Marker Proteins—Relative abundances. Box plots showing the relative abundances calculated by dividing WFDC2 individual abundances by the individual abundances of each of the selected E6 marker proteins. Box plot whiskers represent minimum and maximum values with the exception of outliers (shown as dots). B) Table showing the relative abundances of each protein-pair in each condition and cell line and the corresponding inter-condition fold-change and T-test for the significance of the difference between conditions.

FIG. 33. Box plots of individual abundances of E8 marker proteins in the proteomics data (1), that is proteins that have a greater abundance in the pluripotency medium E8 than in the E6 medium. Box plot whiskers represent minimum and maximum values with the exception of outliers (shown as dots).

FIG. 34. Box plots of individual abundances of E8 marker proteins in the proteomics data (2) that is proteins that have a greater abundance in the pluripotency medium E8 than in the E6 medium. Box plot whiskers represent minimum and maximum values with the exception of outliers (shown as dots).

FIG. 35. Box plots of individual abundances of E8 marker proteins in the proteomics data (3) that is proteins that have a greater abundance in the pluripotency medium E8 than in the E6 medium. Box plot whiskers represent minimum and maximum values with the exception of outliers (shown as dots).

FIG. 36. Box plots of individual abundances of E6 marker proteins in the proteomics data that is proteins that have a greater abundance in the loss of pluripotency medium E6 than in the pluripotency E8 medium. Box plot whiskers represent minimum and maximum values with the exception of outliers (shown as dots).

FIG. 37. Table showing the inter-condition log fold-changes and FDR adjusted p-values for the RNA and protein individual abundances.

FIG. 38. Assessment of the relative abundance of NPTX2 /FST in stem cell cultures cultured in alternative media. A) shows the detection of NPTX2, FST and NID protein in different concentrations of the medium TeSR1. Concentrations of the proteins in E6 medium are also shown as a control. B) shows TESR1 grown cells and cells grown in Stage 1 Chondrocyte differentiation media after 24 and 48 h. The increased Follistin after 24 then 48 h in differentiation medium with loss of NPTX2 is clearly observed. C) shows NPTX2/FST ratio in media concentrated from cells grown in TeSR1 pluripotency media versus Stagel Chondrocyte differentiation media. This shows that a reduction in the relative abundance of NPTX2/FST indicates a loss of pluripotency. This finding is in line with the finding in the media using E8/E6 media as shown in FIG. 24A) and B) thus demonstrating that the relative abundance of protein pairs described herein can be used as an indicator of the pluripotency state using different media.

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous. The practice of the present invention will employ, unless otherwise indicated, conventional techniques of cell biology, microbiology, tissue culture, molecular biology, chemistry, biochemistry, recombinant DNA technology, bioinformatics which are within the skill of the art. Such techniques are explained fully in the literature.

The inventors have identified protein biomarkers in the secretome of pluripotent stem cells which can be used as markers to provide an indication of the pluripotent state of the stem cell culture, e.g. an indication of loss of pluripotency. The biomarkers were identified in a proteomic analysis of the spent medium from cultured embryonic (Man-13) and induced (Rebl.PAT) pluripotent stem cells. Cells were grown in Essential 8 medium (E8), a medium that maintains pluripotency, and then transferred to E6 medium (E6) for 48 hours to replicate an early, undirected dissolution of pluripotency. E6 is identical to E8 medium but lacks the growth factors FGF2 and TGF6 and thus induces differentiation. It can thus be described as a differentiation medium. Therefore, E6 medium can be used to test which proteins are differentially produced in cells that have lost pluripotency compared to cells that are pluripotent. Medium was harvested from both E8 and E6 culture and changes in the transcriptome between the two media were analysed to identify pluripotency markers. Protein biomarkers were identified that are present at different levels in the E8 media (“the pluripotency medium”) and the E6 media (“the differentiation medium”). Thus, protein markers could be determined that are associated with early loss of pluripotency.

Additionally, the ratio of specific proteins pairs was assessed. 4 E8-enriched and 4 E6-enriched proteins which were demonstrated to give 16 protein-pair ratios that consistently and robustly distinguished the two cell states (i.e. pluripotent v. differentiating). The inventors further demonstrated that returning the 48 hour, E6-cultured cells to E8 for 7 days recovered healthy levels of pluripotency and that the ratios of our pluripotency associated markers in the secretome reflected this. Subsequent experiments identified further protein pairs that are biomarkers for the pluripotent state of a cell culture. Assessing the ratio of two proteins, i.e. the relative abundance of the proteins, thus provides a reliable and robust way of assessing the pluripotent state of a stem cell culture. Therefore, according to the methods of the invention, the state of a stem cell culture in any given stem cell line and medium can be monitored by establishing a reference or baseline value of pluripotent cell culture (e.g. a baseline value of the absolute or relative abundance of the proteins) and then monitoring the absolute and/or relative abundance of the proteins. A deviation from the reference value indicates a change of the pluripotency state. The terms reference or baseline value are used interchangeably herein.

In a first aspect, the invention thus relates to a method for determining the pluripotent state of a stem cell culture; said method comprising culturing stem cells and

a) measuring the relative abundance of two proteins selected from: Argininosuccinate synthetase 1 (ASS1), Creatine kinase B-type (CKB), chondromodulin-1 (CNMD), Cochlin (COCH), Fibroblast Growth Factor Receptor 1 (FGFR1), Follistatin (FST), Olfactomedin-like 3 (OLFML3), Serpin B1 (SERPINB1), Stomatin (STOM), A disintegrin and metalloproteinase with thrombospondin motifs 8 (ADAMTS8), Amyloid Beta Precursor Protein (APP), UDP-GlcNAc:BetaGal Beta-1,3-N-Acetylglucosaminyltransferase 7 (B3GNT7), Chromogranin A (CHGA), EGF-containing fibulin-like extracellular matrix protein 1 (EFEMP1), Ephrin type-A receptor 1 (EPHA1), Exostosin-like 2 (EXTL2), Fibulin 2 (FBLN2), Fibroblast Growth Factor 2 (FGF2), Plasma alpha-L-fucosidase (FUCA2), Polypeptide N-acetylgalactosaminyltransferase 1 (GALNT), Gamma-Glutamyl Hydrolase (GGH), Beta-hexosaminidase subunit beta (HEXB), Insulin-like growth factor-binding protein 4 (IGFBP4), Nidogen 1 (NID1), Neuronal Pentraxin-2 (NPTX2), Neurotensin (NTS), Protocadherin beta-5 (PCDHB5), Semaphorin 3A (SEMA3A), Semaphorin 3F (SEMA3F), Secreted Frizzled Related Protein 2 (SFRP2), Metalloproteinase inhibitor 4 (TIMP4), Tumor necrosis factor receptor superfamily member 8 (TNFRSF8) and WAP four-disulfide core domain protein 2 (WFDC2) and comparing said relative abundance to a reference value and/or

b) measuring the absolute abundance of one or more protein selected from Argininosuccinate synthetase 1 (ASS1), Creatine kinase B-type (CKB), chondromodulin-1 (CNMD), Cochlin (COCH), Fibroblast Growth Factor Receptor 1 (FGFR1), Follistatin (FST), Olfactomedin-like 3 (OLFML3), Serpin B1 (SERPINB1), Stomatin (STOM), A disintegrin and metalloproteinase with thrombospondin motifs 8 (ADAMTS8), Amyloid Beta Precursor Protein (APP), UDP-GlcNAc:BetaGal Beta-1,3-N-Acetylglucosaminyltransferase 7 (B3GNT7), Chromogranin A (CHGA), EGF-containing fibulin-like extracellular matrix protein 1 (EFEMP1), Ephrin type-A receptor 1 (EPHA1), Exostosin-like 2 (EXTL2), Fibulin 2 (FBLN2), Fibroblast Growth Factor 2 (FGF2), Plasma alpha-L-fucosidase (FUCA2), Polypeptide N-acetylgalactosaminyltransferase 1 (GALNT), Gamma-Glutamyl Hydrolase (GGH), Beta-hexosaminidase subunit beta (HEXB), Insulin-like growth factor-binding protein 4 (IGFBP4), Nidogen 1 (NID1), Neuronal Pentraxin-2 (NPTX2), Neurotensin (NTS), Protocadherin beta-5 (PCDHB5), Semaphorin 3A (SEMA3A), Semaphorin 3F (SEMA3F), Secreted Frizzled Related Protein 2 (SFRP2), Metalloproteinase inhibitor 4 (TIMP4), Tumor necrosis factor receptor superfamily member 8 (TNFRSF8) and WAP four-disulfide core domain protein 2 (WFDC2) and comparing said absolute abundance to a reference value.

As used herein the term “stem cell” (“SC”) refers to cells that can self-renew and differentiate into multiple lineages. A stem cell is a developmentally pluripotent or multipotent cell. A stem cell can divide to produce two daughter stem cells, or one daughter stem cell and one progenitor (“transit”) cell, which then proliferates into the tissue's mature, fully formed cells. Stem cells may be derived, for example, from embryonic sources (“embryonic stem cells”) or derived from adult sources. According to the invention, different stem cell lines may be used, including but not limited to Man-13 and Rebl.PAT.

Examples of adult stem cells include, but are not limited to, hematopoietic stem cells, neural stem cells, mesenchymal stem cells, and bone marrow stromal cells. These stem cells have demonstrated the ability to differentiate into a variety of cell types including adipocytes, chondrocytes, osteocytes, myocytes, bone marrow stromal cells, and thymic stroma (mesenchymal stem cells); hepatocytes, vascular cells, and muscle cells (hematopoietic stem cells); myocytes, hepatocytes, and glial cells (bone marrow stromal cells) and, indeed, cells from all three germ layers (adult neural stem cells).

As used herein, the term “pluripotent cell” or “pluripotent stem cell” (PSCs) refers to a cell that has complete differentiation versatility, e.g., the capacity to grow into any of the mammalian body's approximately 260 cell types. A pluripotent cell can be self-renewing in vitro and transiently in the embryo,. The term pluripotent stem cell includes induced pluripotent stem cells reprogrammed from somatic cells to have pluripotent stem cell properties. Unlike a totipotent cell (e.g., a fertilized, diploid egg cell), a pluripotent cell, even a pluripotent embryonic stem cell, cannot usually form a new blastocyst. According to the various aspects of the invention, the pluripotent stem cell may be a human pluripotent stem cell.

Mammalian embryonic stem (ES) cells are pluripotent cells derived from the inner cell mass of a blastocyst. ES cells can be isolated by removing the outer trophectoderm layer of a developing embryo, then culturing the inner mass cells on a feeder layer of non-growing cells. Under appropriate conditions, colonies of proliferating, undifferentiated ES cells are produced. The colonies can be removed, dissociated into individual cells, then replated on a fresh feeder layer. The replated cells can continue to proliferate, producing new colonies of undifferentiated ES cells. The new colonies can then be removed, dissociated, replated again and allowed to grow. This process of “subculturing” or “passaging” undifferentiated ES cells can be repeated a number of times to produce cell lines containing undifferentiated ES cells.

As used herein, the term “embryonic stem cell” (“ES cell” or ESC”) refers to a pluripotent cell that is derived from the inner cell mass of a blastocyst (e.g., a 4- to 5-day-old human embryo), and has the ability to yield many or all of the cell types present in a mature animal. Non-limiting examples of human embryonic stem cells include HI, H9, hES2, hES3, hES4, hESS, hES6, BGOI, BG02, BG03, HSFI, HSF6, HI, H7, H9, H13B, MAN1-16 and H14. The pluripotent cells may be induced pluripotent cells (iPSC), as discussed in greater detail below. Non-limiting examples of iPSC include ReblPat.

Methods for obtaining mouse ES cells are well known. In one method, a preimplantation blastocyst from the 129 strain of mice is treated with mouse antiserum to remove the trophoectoderm, and the inner cell mass is cultured on a feeder cell layer of chemically inactivated mouse embryonic fibroblasts in medium containing fetal calf serum. Colonies of undifferentiated ES cells that develop are subcultured on mouse embryonic fibroblast feeder layers in the presence of fetal calf serum to produce populations of ES cells. In some methods, mouse ES cells can be grown in the absence of a feeder layer by adding the cytokine leukemia inhibitory factor (LIF) to serum-containing culture medium.

Another source of ES cells are established ES cell lines. Various mouse cell lines and human ES cell lines are known and conditions for their growth and propagation have been defined in the art. By no means does the practice of this invention require that a human blastocyst be disaggregated in order to produce the hES or embryonic stem cells for practice of this invention. Indeed, hES cells can be obtained from established lines obtainable from public depositories (for example, the WiCell Research Institute, Madison Wis. U.S.A., or the American Type Culture Collection, Manassas Va., U.S.A.).

The PSCs may be “induced pluripotent stem cells” (“iPSCs”), that is stem cells induced by reprogramming from a somatic cell, e.g., a differentiated somatic cell. iPS cells are capable of self-renewal and differentiation into mature cells in a similar way to ESCs.

As used herein the term “feeder cells” refers to cells used as a growth support in some tissue culture systems. Feeder cells may be embryonic striatum cells or stromal cells.

The present invention is not limited to certain culture media used for culturing the stem cells and a skilled person would know that a number of different media as disclosed in the art can be used. Suitable medium supports the pluripotency and maintain self-renewal of the PSCs. In certain aspects, cells may be grown with feeder cells such a fibroblasts or in fibroblast conditioned media. However, preferably, it may be preferred that stem cells are grown in the absence of feeder cells. In some aspects, cells may be grown in a defined media such as TeSR1 media (see US2006/084168). Such media may be used for serum free culture of ES cells. In some embodiments, media is supplemented with bovine or human serum to supply the necessary growth factors.

For example, the culture medium can be selected from DMEM/F12, RPMI 1640, GMEM, or neurobasal medium, TeSR1, TeSR2, TeSR-E8, RSeT and NaiveCult (STEMCELL Technologies), Stem Pro, Essential 8 (Thermo Fisher Scientific), StemFlex (Thermo Fisher Scientific), Pluripro (Cell Guidance Systems), PluriSTEM (Millipore), StemFit (Ajinomoto), or Nutristem (Corning). In one embodiment, the culture media is Essential 8 medium (E8). Some of these media such as TeSR2 contain human serum albumin (HSA), whereas the more recent and widely used E8 medium, which is derived from TeSR2, does not contain either HSA or bovine serum albumin (BSA). E8 medium is a xeno-free and feeder-free medium specially formulated for the growth and expansion of human pluripotent stem cells [18].

The stem cell culture medium can contain serum, or can be a serum- free medium. The serum-free medium can be used without the addition of an exogenous growth factor, or can be supplemented with a growth factor such as basic fibroblast growth factor (bFGF), insulin-like growth factor-2 (IGF-2), epidermal growth factor (EGF), fibroblast growth factor 8 (FGF8), Sonic hedgehog (Shh), brain derived neurotrophic factor (BDNF), glial cell line-derived neurotrophic factor (GDNF), or Vitamin C. The non-adherent surface can be low-attachment tissue culture plastic.

The media may comprise additional factors, such as a Rock inhibitor. It is known that inhibiting Rho kinase (ROCK) activity with a Rock inhibitor such as Y-27632, significantly enhances recovery of hES cells from cryopreserved stocks and increases the number of colonies and colony size of hES cells. The culture media may also contain salts, vitamins, amino acids, glucose, a fibroblast growth factor, gamma amino butyric acid, pipecholic acid, lithium and transforming growth factor beta, in sufficient amounts to maintain the human stem cells in an undifferentiated state through multiple successive culture passages.

The terms “absolute abundance” or “individual abundance” as used herein refers to the level, i.e. concentration of an individual protein of interest in a test sample, i.e. in a stem cell culture. Therefore, this represents the quantity of an individual protein present in a sample. It can be determined by methods known in the art which are further exemplified below and include Mass spec based methods. The value can be a number provided by either ProgenesisIQ proteomics software [1] or ImageJ image analysis software [2] which represents the quantity of an individual protein

The term “relative abundance” as used herein refers to the abundance of protein X /abundance of protein Y in same sample. Thus, to assess relative abundance, a protein pair is analysed. Relative abundance is calculated by dividing protein X individual abundance by the individual abundance of protein Y. This is further illustrated in the examples and figures. Thus, the relative abundance value represents the difference in abundance between two proteins of interest. Measuring relative protein abundance provides a more reliable, statistically significant and reproducible comparison compared to measuring absolute abundance of individual proteins. By assessing relative abundance, marker expression is “normalised”, which compensates for intra- and inter-kinetic variations (sample-to-sample and run-to-run variations). Normalised data are particularly useful when quantitating protein expression. The method of the invention thus does not rely on quantitative expression of protein (as in disease body fluid biomarker discovery and assessment) but rather it relies on ratios between controlled conditions applied to individual cell lines: the pluripotent maintenance condition and an early spontaneous or induced differentiation induction condition. The method thus substantially avoids the difficulties inherent in quantitation of novel (e.g. disease) biomarkers in body fluids/tissue and comparing between completely different individuals with different sex, age, environmental influences and co morbidities etc where modifications and isoforms will obscure/distort data. Median (not mean) based statistics designating differences between the peptide ratios using the median (i.e. not relying on just one ion or an average) under different conditions are used in the calculations.

The terms “reference value” or “baseline value” as used herein refer to a value established in a stem cell reference culture. A “reference culture” may be a positive control or a negative control. When the reference culture is a positive control, the reference culture of cells comprises PSCs. Preferably, such a reference culture comprises a known proportion of PSCs. Thus, depending on the protein marker tested, lower marker level in the culture of cells being tested than marker level in the reference culture of cells indicates absence or presence of residual, undifferentiated PSCs in the cultured cells. In one example, lower marker level in the culture of cells being tested than marker level in the reference culture of cells indicates presence of residual, undifferentiated PSCs in the cultured cells, but presence at a proportion lower than the known proportion of PSCs in the reference culture of cells. The reference culture comprises the same cell line as the test culture and is cultured using the same media as the test culture. For example, the test culture may be derived from the reference culture where the reference culture has been maintained in culture for a period of time so as to assess whether the cells have lost their pluripotency. Pluripotency of the reference culture to establish the pluripotency of the reference culture can be assessed using standard methods by flow cytometry [e.g. using Tra 181 and Nanog], immuno-cytochemistry, culturing cells on, to evaluate with growth and further passage their response to differentiation cues and 3 germ layer embryoid body formation.

According to the method of the invention, a deviation away from the reference value may indicate the loss of pluripotency or potential loss of pluripotency or recovery of pluripotency as explained further herein.

The term “inter-condition log fold-change” is intended to mean a number representing the difference in protein abundance between the two conditions of interest, for example a culture in E8 and E6 cell medium.

The term “pluripotent state” as used herein refers to pluripotency of stem cells in the stem cell culture; i.e. whether the stem cells are pluripotent or not.

The terms test protein or test protein pair refer to the protein /protein pair of interest which is being analysed. Similarly, the term test culture refers to the stem cell culture of interest which is being assessed for its pluripotency state. In one embodiment, the method comprises measuring the relative abundance of one or more protein pair wherein the protein pair is formed by two proteins listed in a single group selected from one of the following groups:

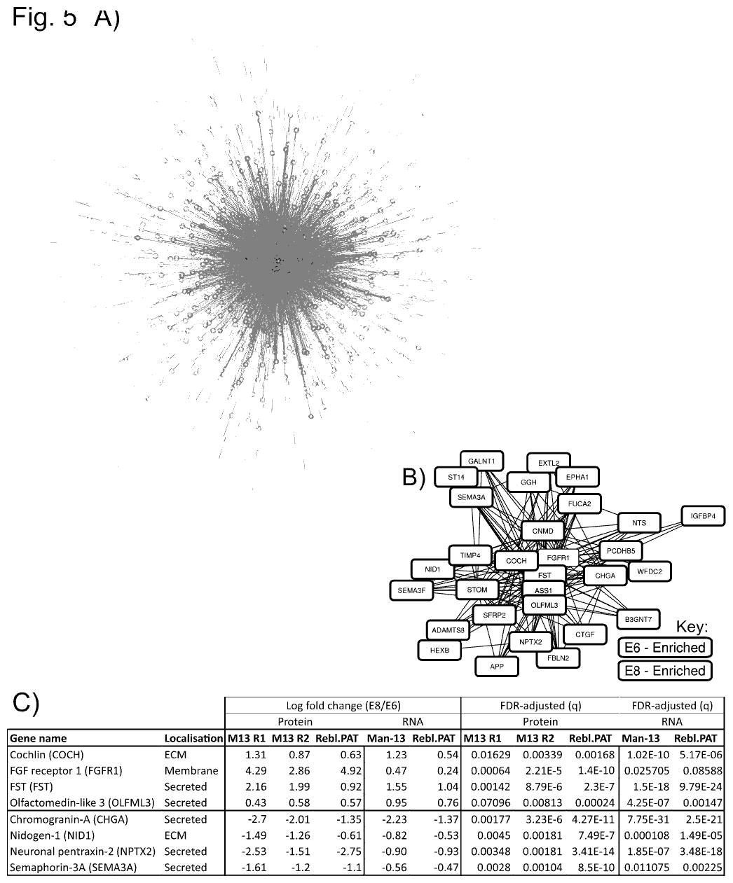

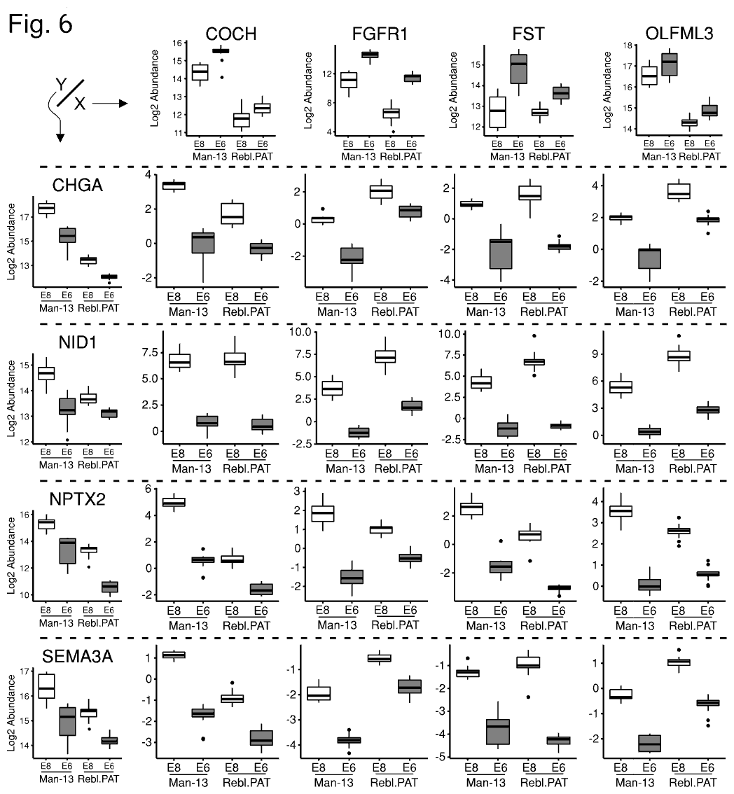

a) COCH, FGFR1, FST, OLFML3, CHGA, NID1, NPTX2, SEMA3A (i.e. 16 possible pairs as shown in FIG. 6), preferably a first protein is selected from a first set of proteins comprising: COCH, FGFR1, FST and OLFML3; and a second protein is selected from a second set of proteins comprising: CHGA, NID1, NPTX2 and SEMA3A

b) ADAMTS8 paired with ASS1, CKB, CNMD, COCH, FGFR1, FST, OLFML3, SERPINB9 or STOM;

c) APP paired with ASS1, CKB, CNMD, COCH, FGFR1, FST, OLFML3, SERPINB9 or STOM;

d) B3GNT7 paired with ASS1, CKB, CNMD, COCH, FGFR1, FST, OLFML3, SERPINB9 or STOM;

e) CHGA paired with ASS1, CKB, CNMD, COCH, FGFR1, FST, OLFML3, SERPINB9 or STOM;

f) EFEMP1 paired with ASS1, CKB, CNMD, COCH, FGFR1, FST, OLFML3, SERPINB9 or STOM;

g) EPHA1 paired with ASS1, CKB, CNMD, COCH, FGFR1, FST, OLFML3, SERPINB9 or STOM;

h) EXTL2 paired with ASS1, CKB, CNMD, COCH, FGFR1, FST, OLFML3, SERPINB9 or STOM;

i) FBLN2 paired with ASS1, CKB, CNMD, COCH, FGFR1, FST, OLFML3, SERPINB9 or STOM;

j) FGF2 paired with ASS1, CKB, CNMD, COCH, FGFR1, FST, OLFML3, SERPINB9 or STOM;

k) FUCA2 paired with ASS1, CKB, CNMD, COCH, FGFR1, FST, OLFML3, SERPINB9 or STOM;

l) GALNT1 paired with ASS1, CKB, CNMD, COCH, FGFR1, FST, OLFML3, SERPINB9 or STOM;

m) GGH paired with ASS1, CKB, CNMD, COCH, FGFR1, FST, OLFML3, SERPINB9 or STOM;

n) HEXB paired with ASS1, CKB, CNMD, COCH, FGFR1, FST, OLFML3, SERPINB9 or STOM;

o) IGFBP4 paired with ASS1, CKB, CNMD, COCH, FGFR1, FST, OLFML3, SERPINB9 or STOM;

p) NID1 paired with ASS1, CKB, CNMD, COCH, FGFR1, FST, OLFML3, SERPINB9 or STOM;

q) NPTX2 paired with ASS1, CKB, CNMD, COCH, FGFR1, FST, OLFML3, SERPINB9 or STOM;

r) NTS paired with ASS1, CKB, CNMD, COCH, FGFR1, FST, OLFML3, SERPINB9 or STOM;

s) PCDHB5 paired with ASS1, CKB, CNMD, COCH, FGFR1, FST, OLFML3, SERPINB9 or STOM;

t) SEMA3A paired with ASS1, CKB, CNMD, COCH, FGFR1, FST, OLFML3, SERPINB9 or STOM;

u) SEMA3F paired with ASS1, CKB, CNMD, COCH, FGFR1, FST, OLFML3, SERPINB9 or STOM;

v) SFRP2 paired with ASS1, CKB, CNMD, COCH, FGFR1, FST, OLFML3, SERPINB9 or STOM;

w) TIMP4 paired with ASS1, CKB, CNMD, COCH, FGFR1, FST, OLFML3, SERPINB9 or STOM;

x) TNFRSF8 paired with ASS1, CKB, CNMD, COCH, FGFR1, FST, OLFML3, SERPINB9 or STOM;

y) WFDC2 paired with ASS1, CKB, CNMD, COCH, FGFR1, FST, OLFML3, SERPINB9 or STOM.

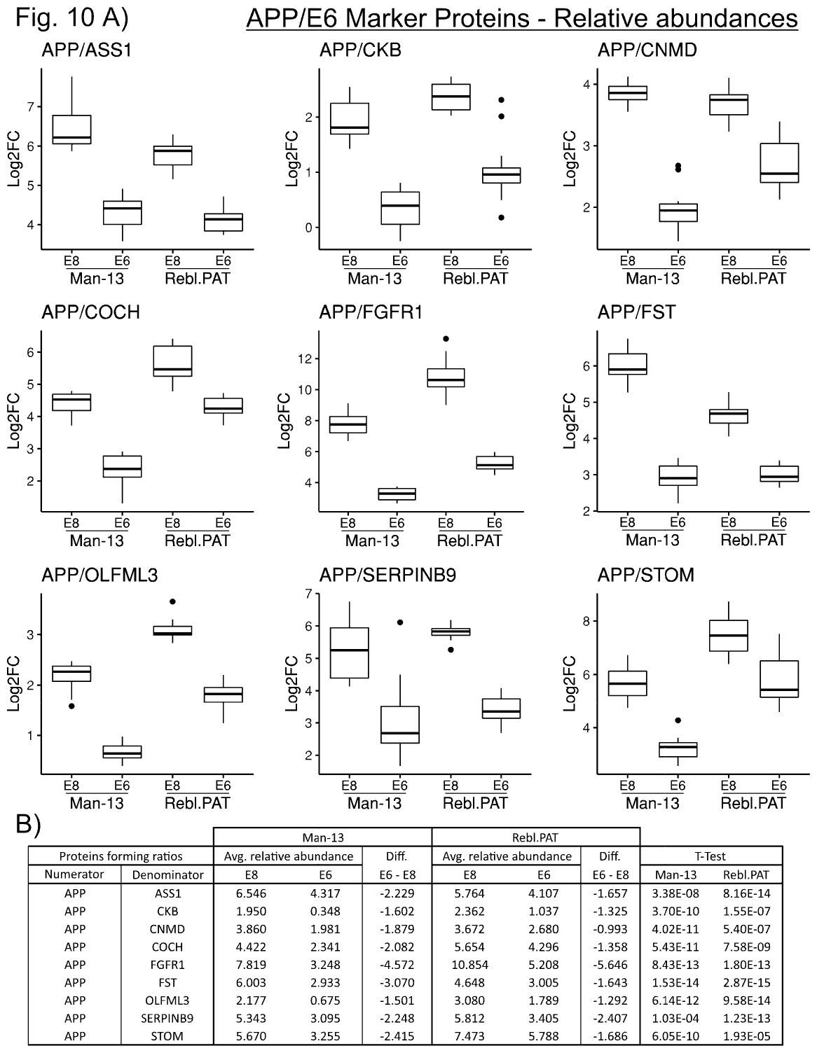

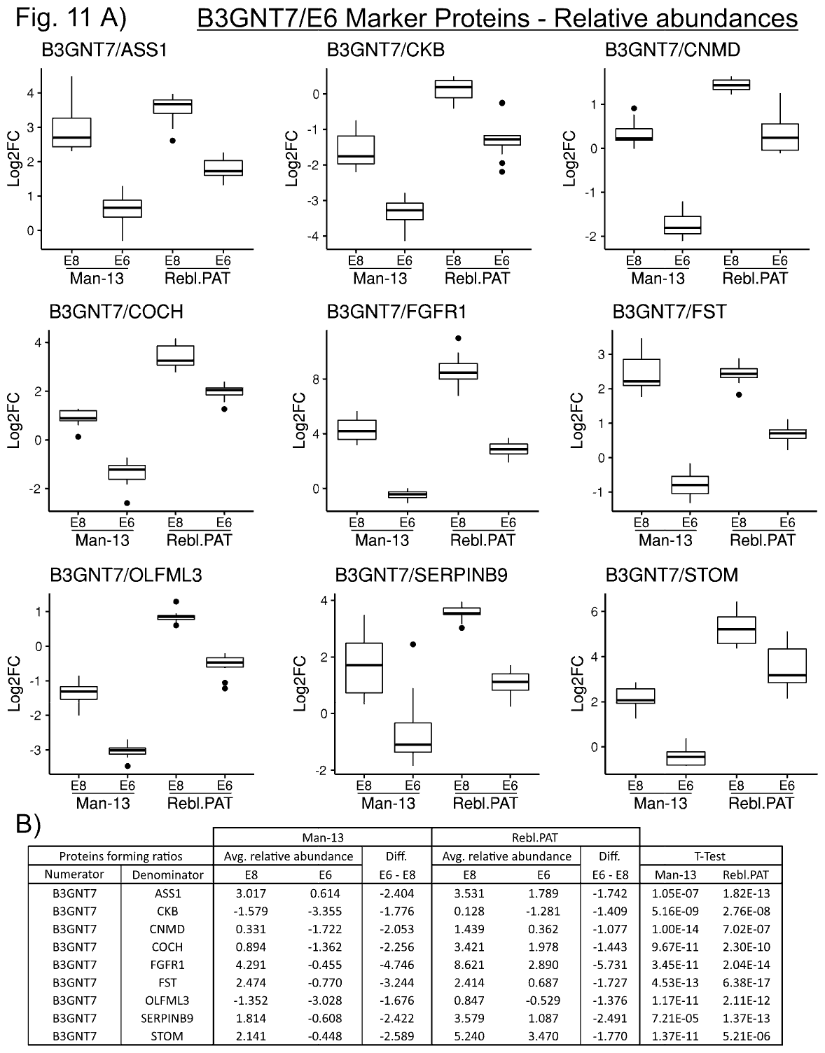

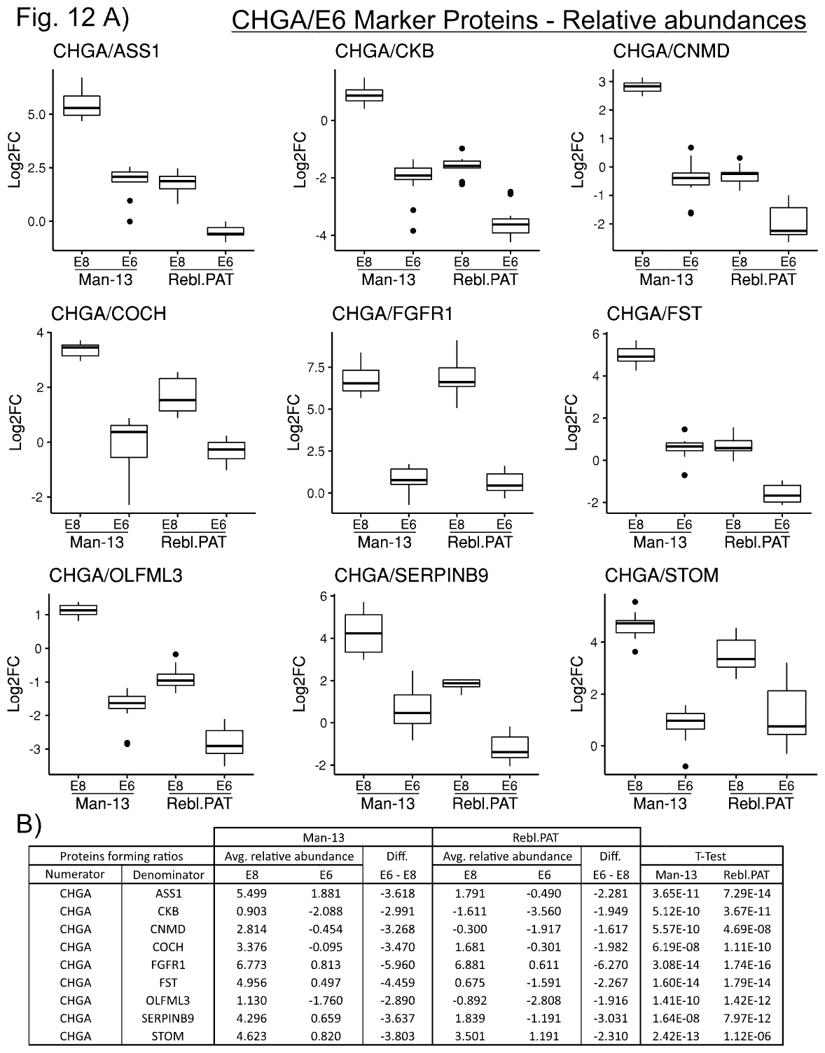

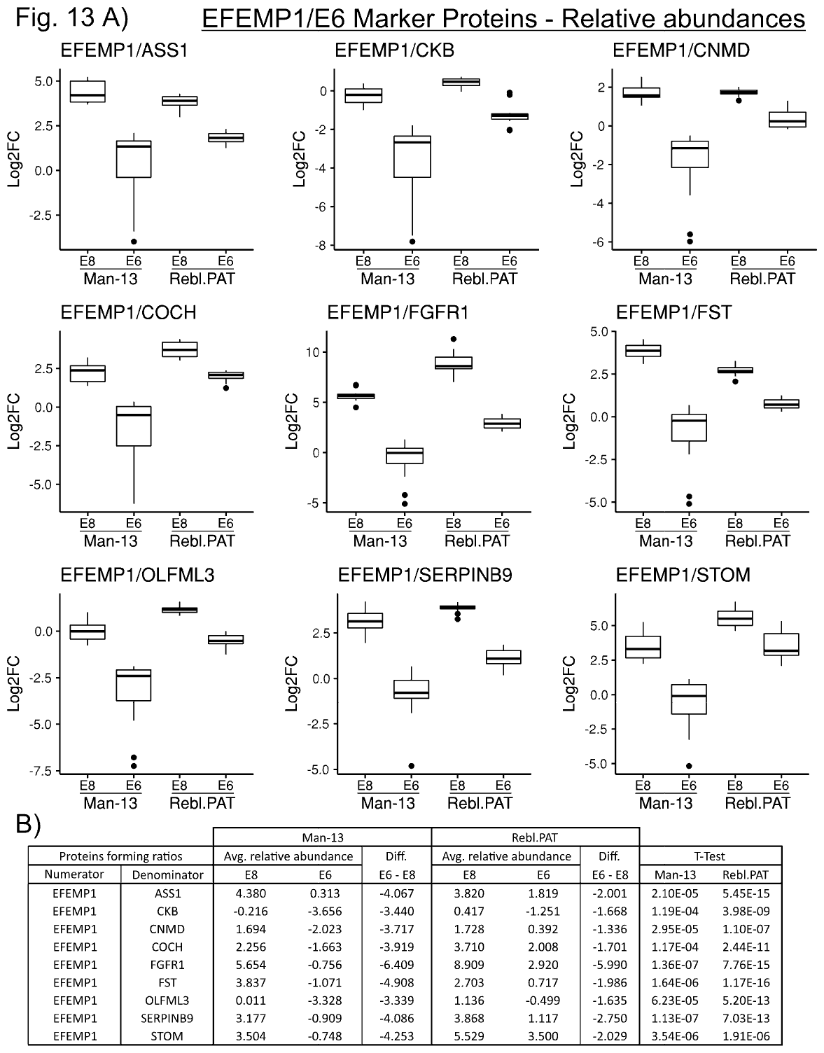

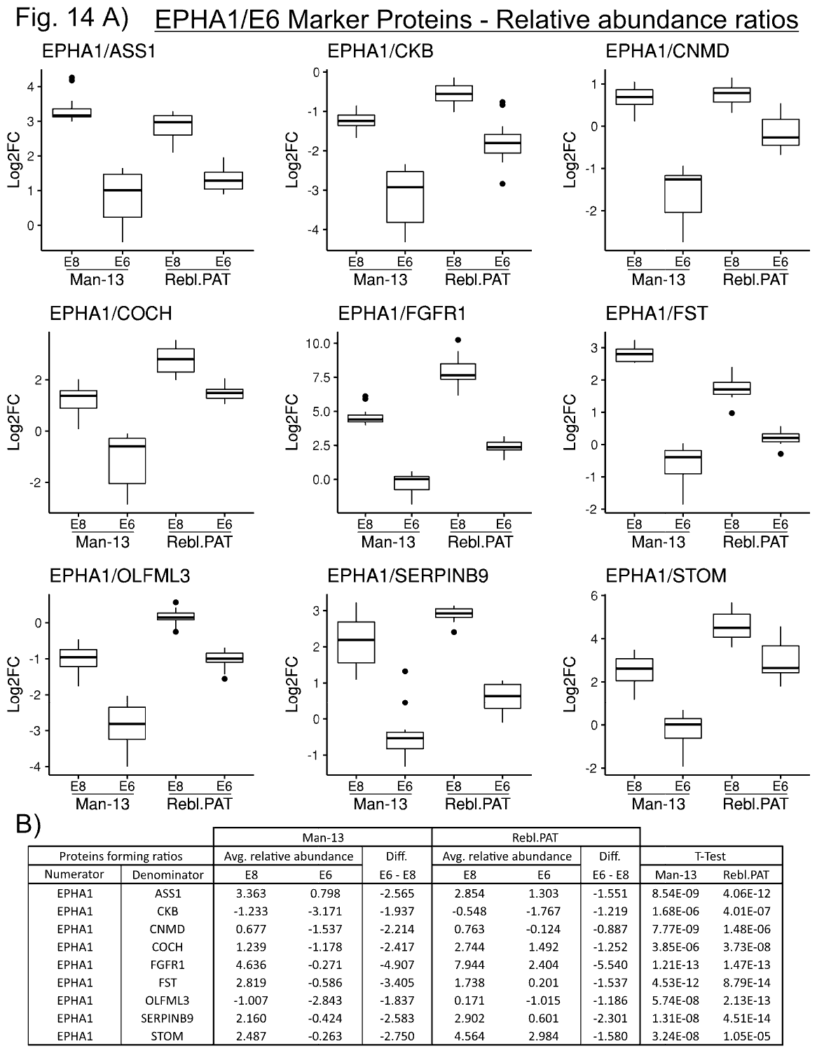

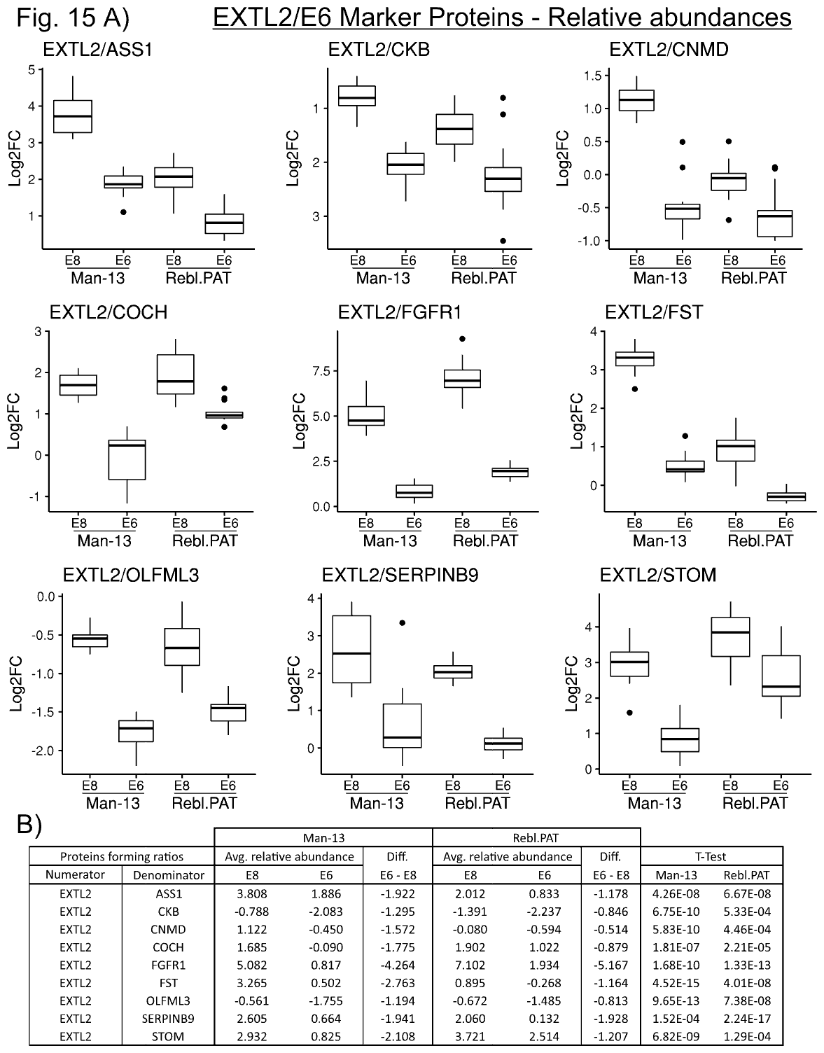

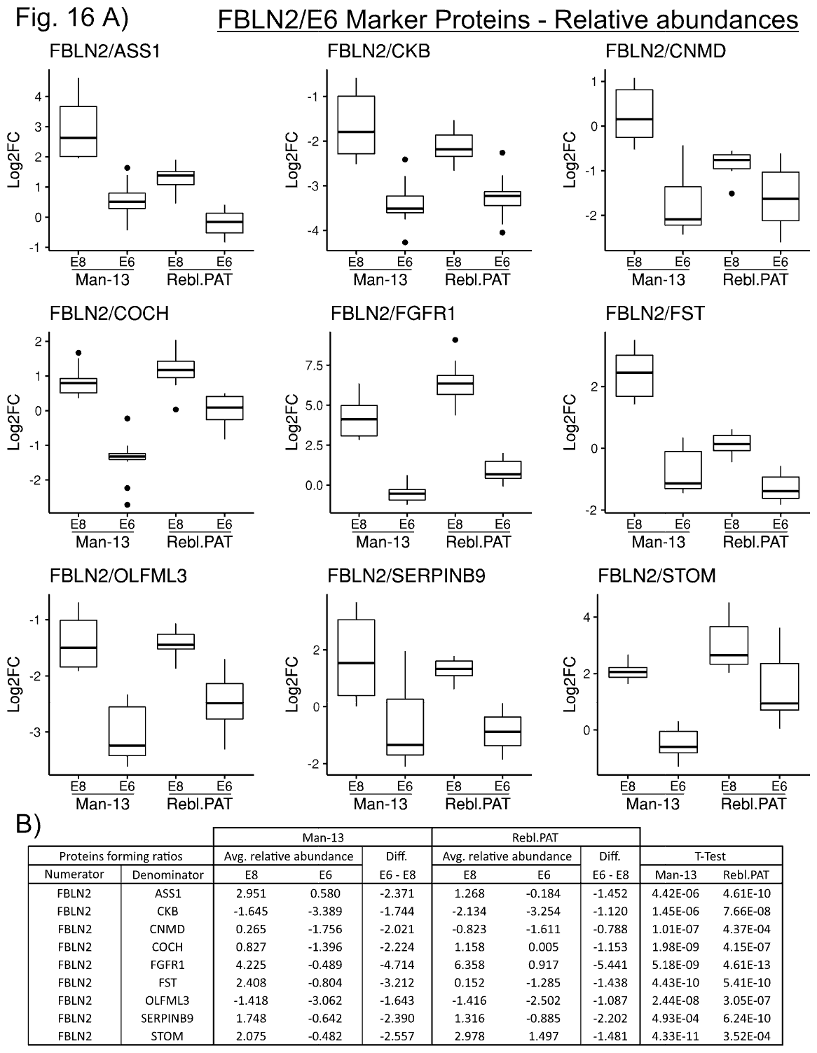

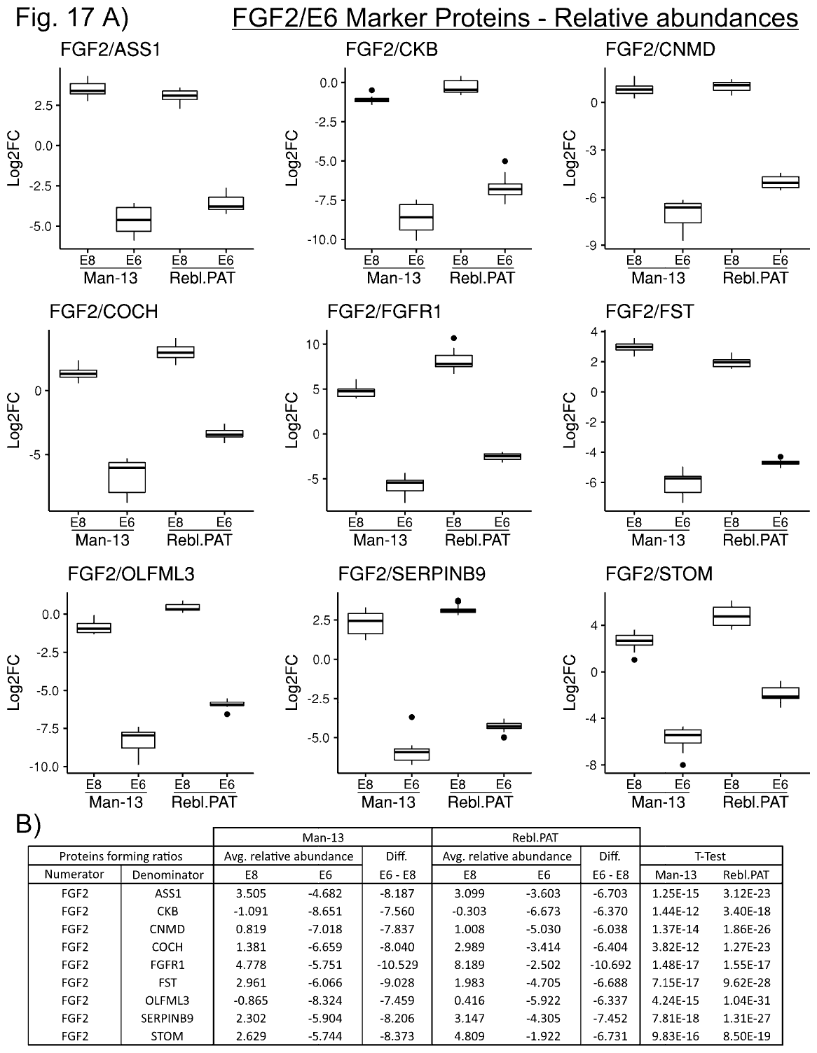

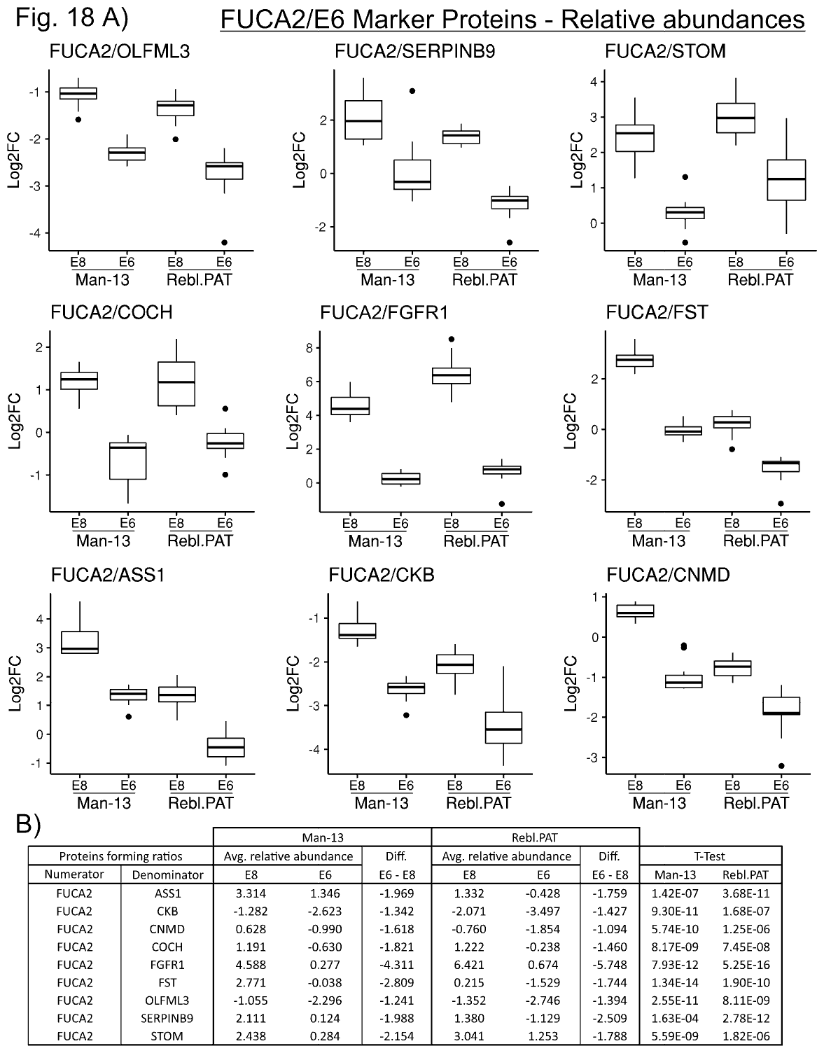

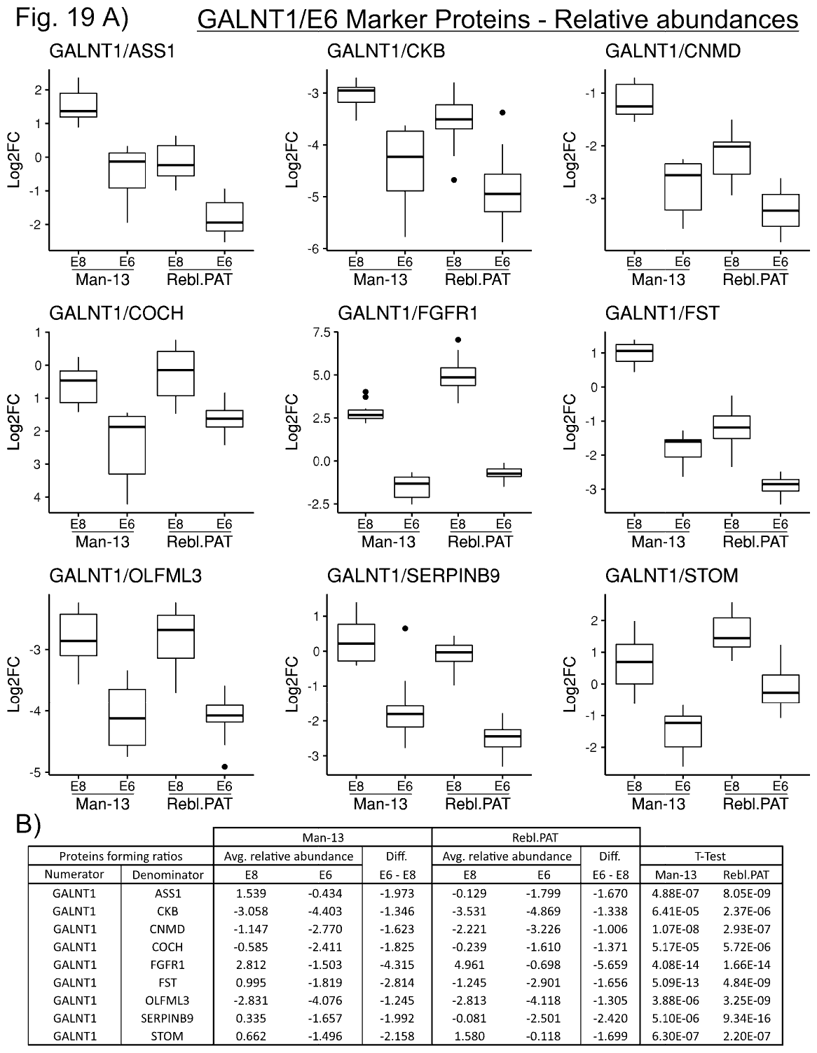

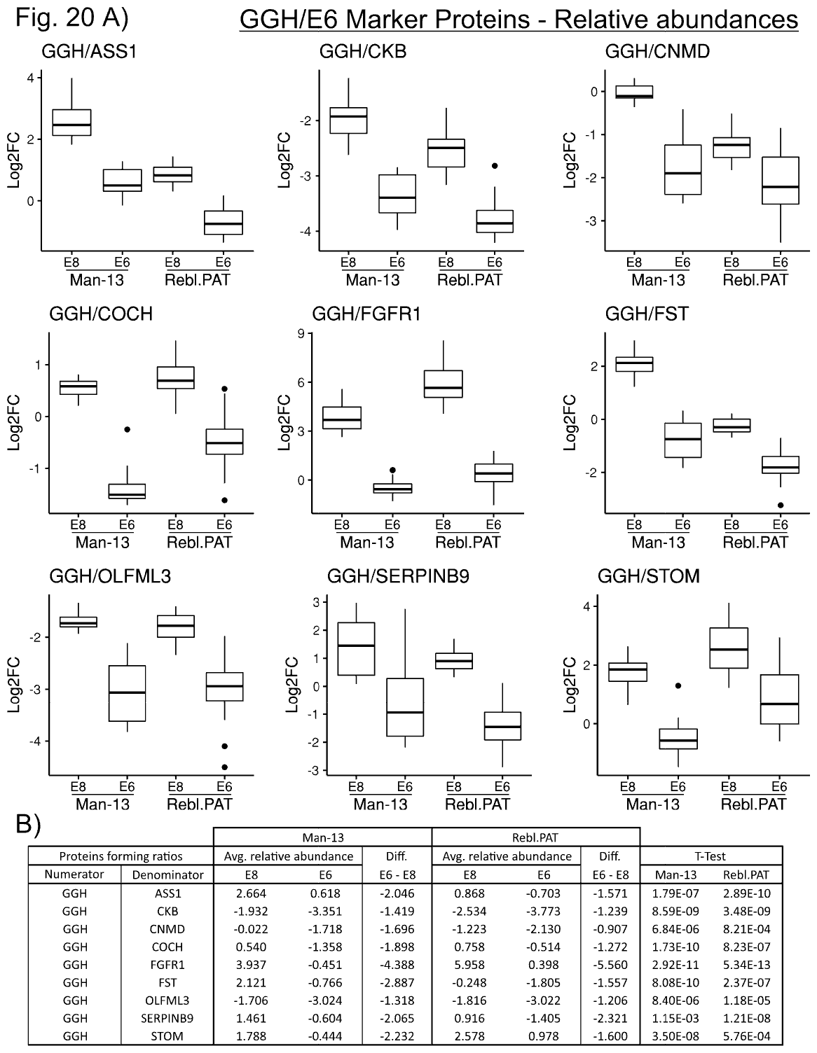

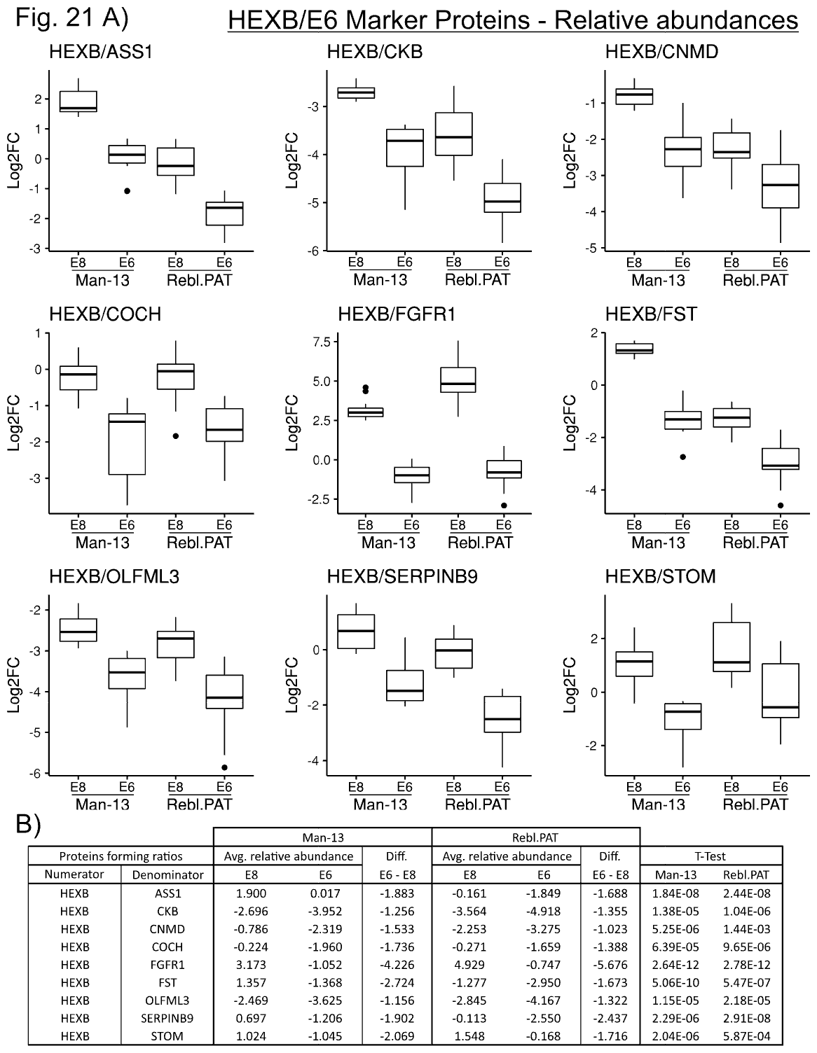

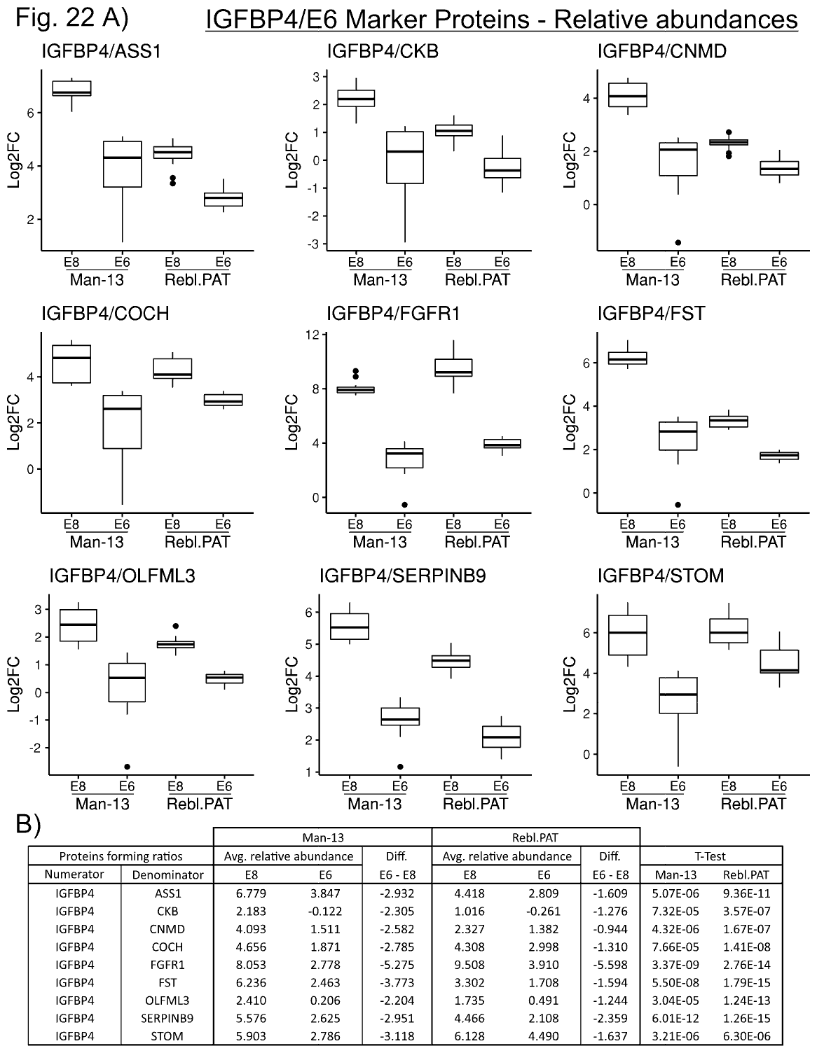

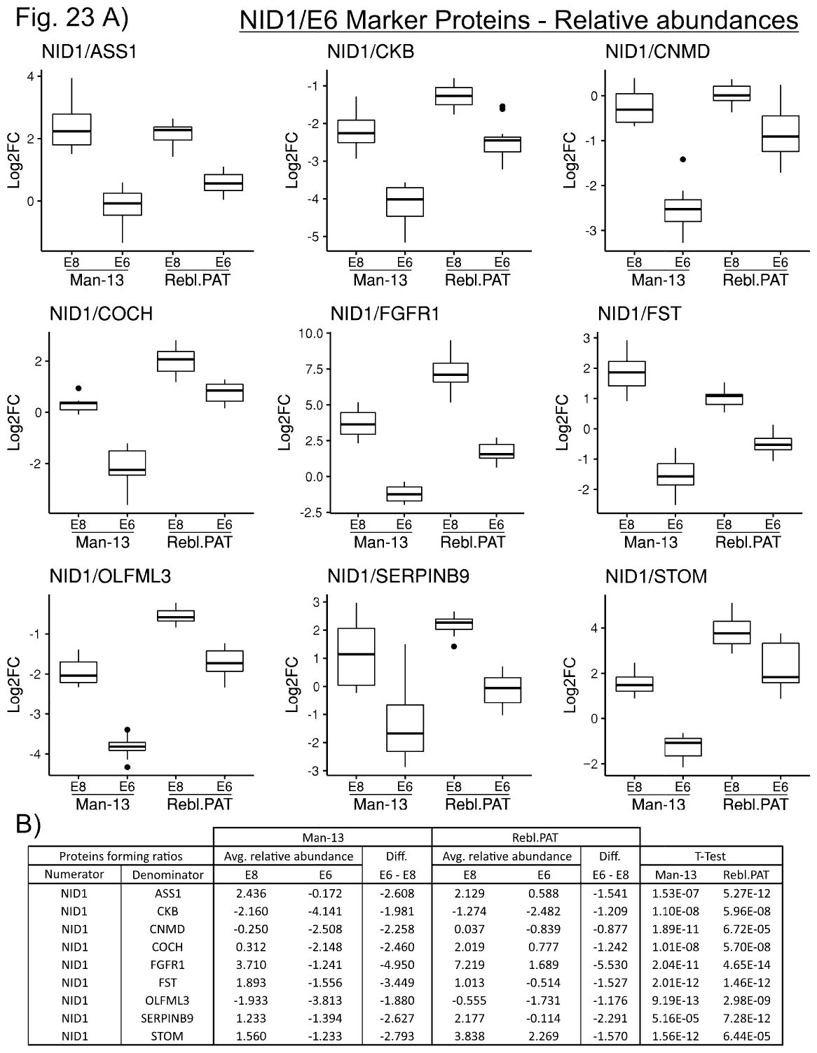

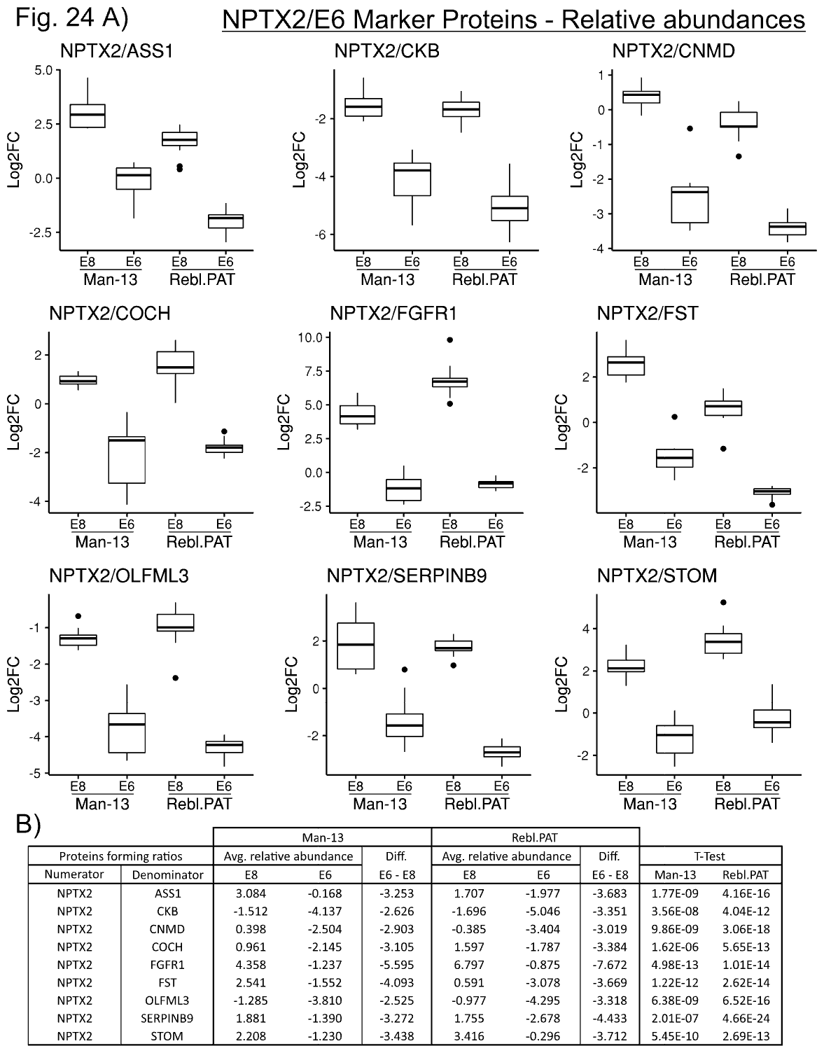

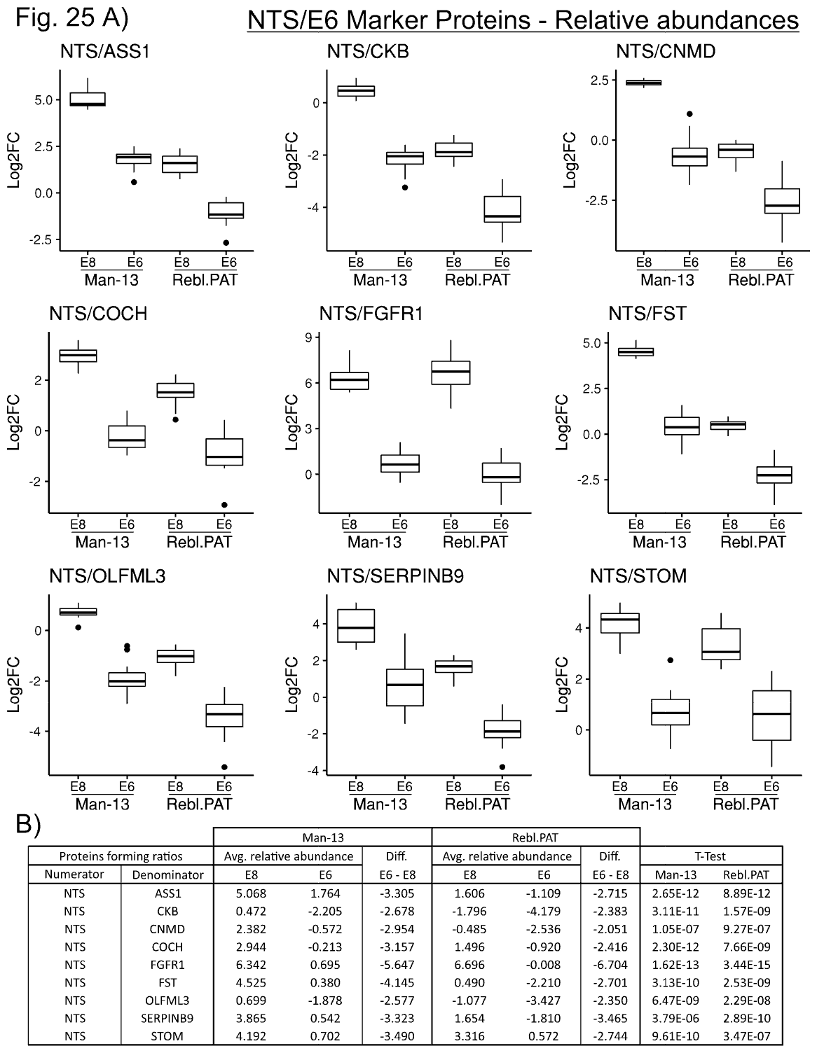

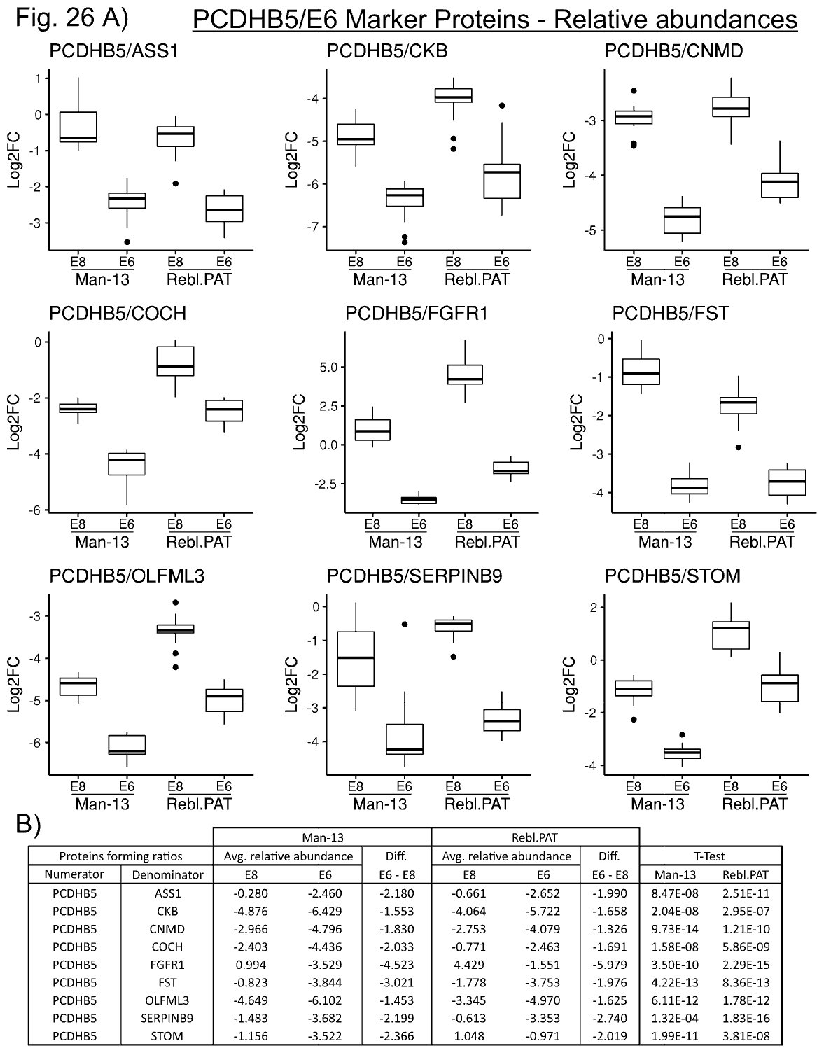

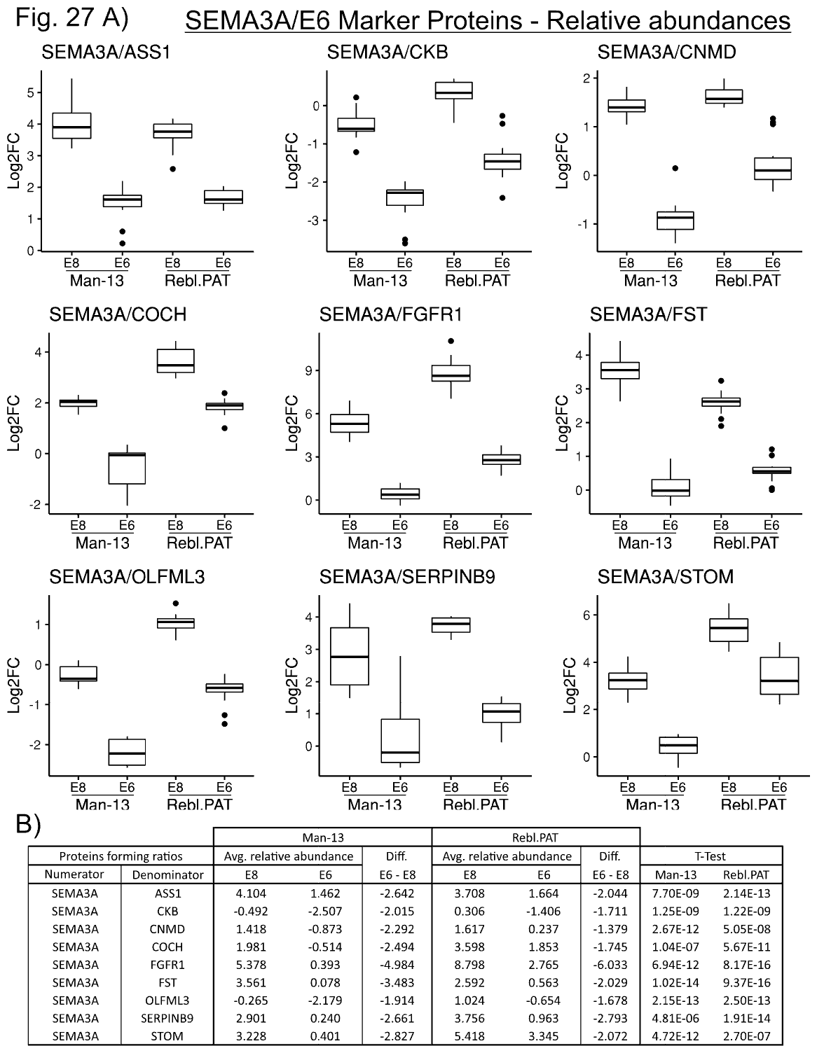

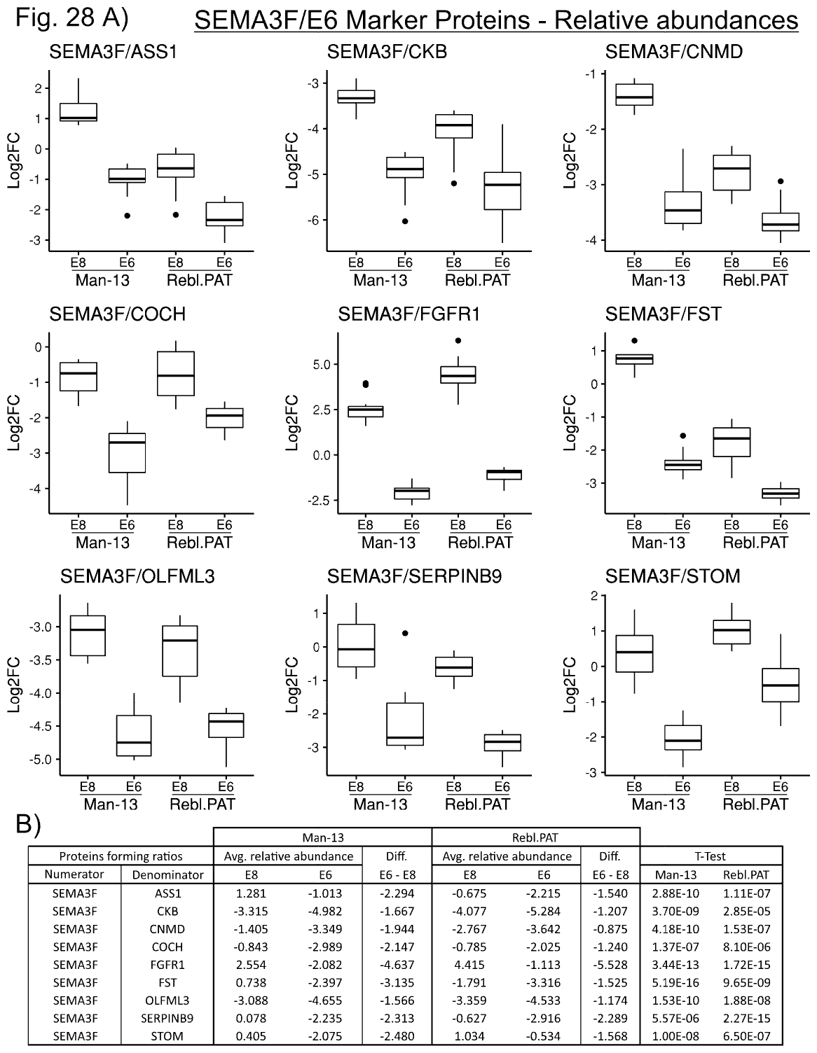

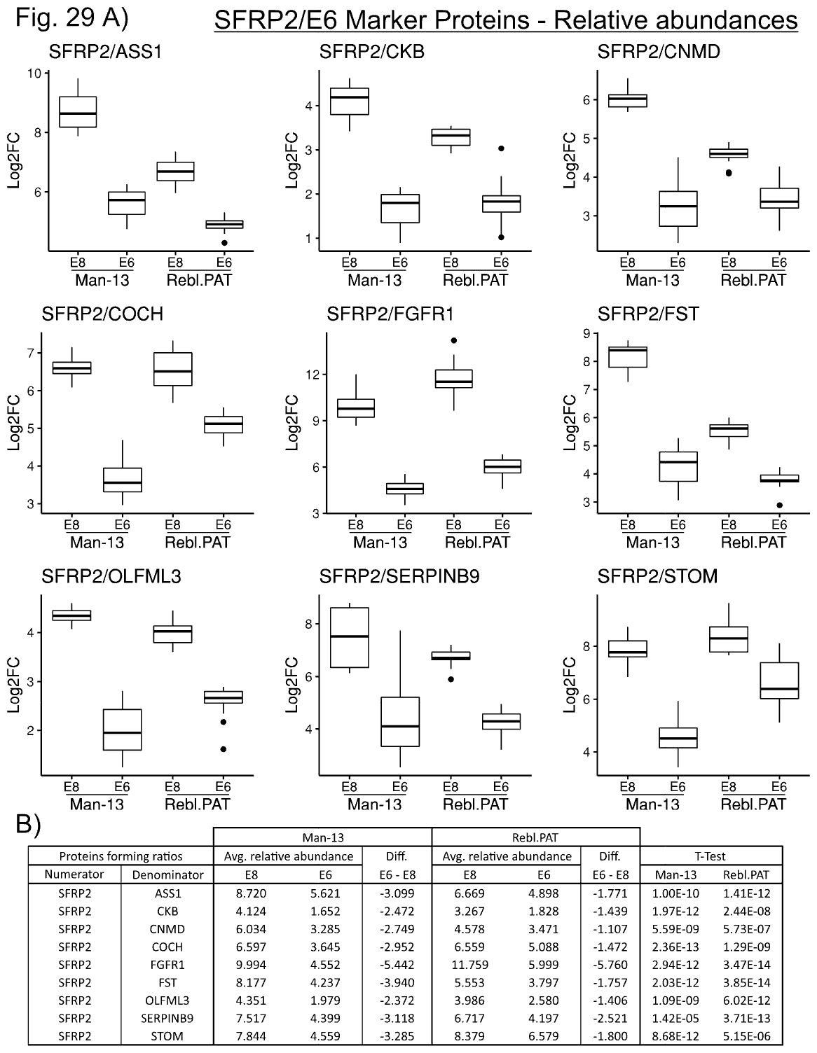

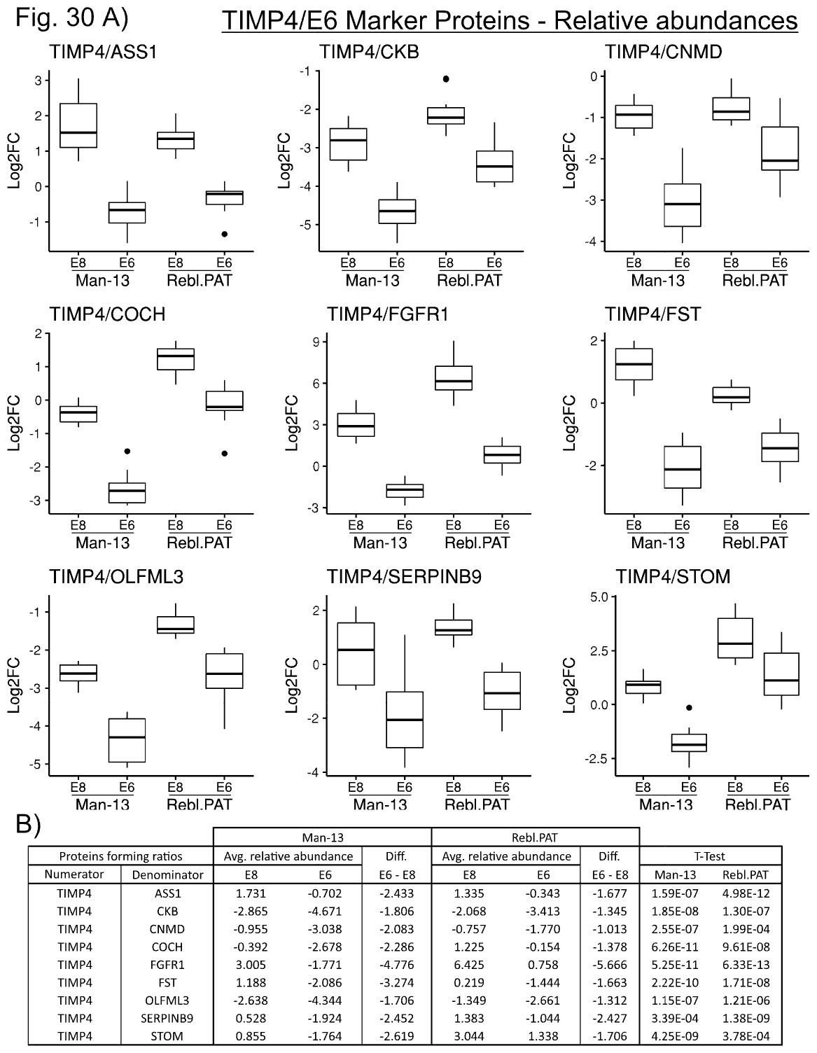

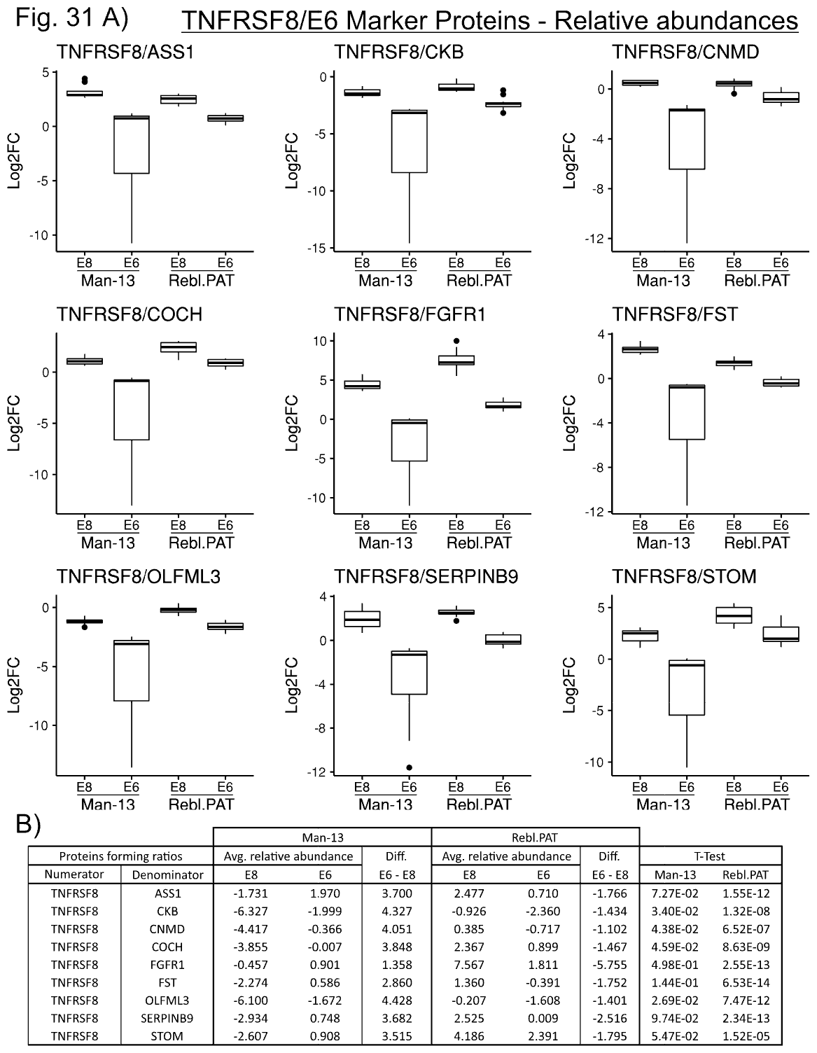

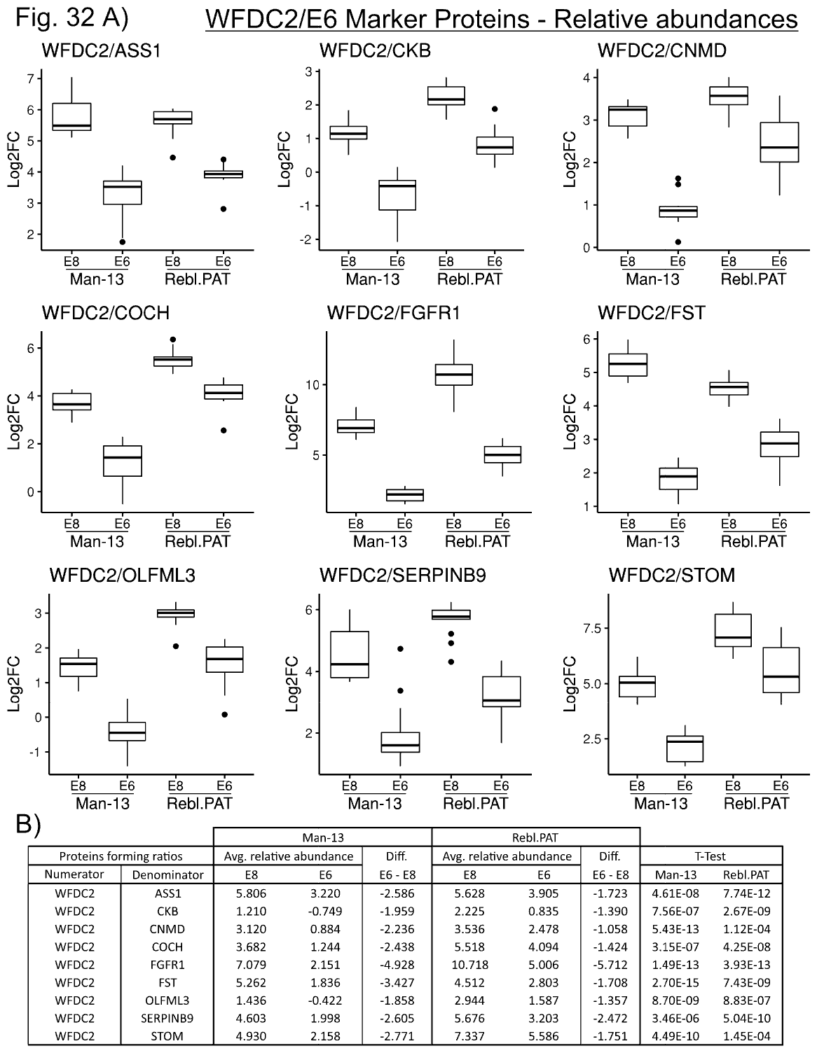

The relative abundance of two proteins measured is compared to a reference value. In one embodiment, the reference value is the relative abundance of the two proteins as measured in a pluripotent stem cell (PSCs) culture, that is a stem cell culture comprising a known proportion of PSCs, preferably a stem cell culture comprising a high proportion of PSCs or more preferably a stem cell culture comprising a pure culture of pristine pSCs. The difference in relative abundance indicates whether the stem cells have lost pluripotency, that is whether the tested stem cells comprise undifferentiated cells at a proportion lower than the proportion of PSCs in the reference culture of cells (and thus differentiated cells at a proportion higher than the proportion of PSCs in the reference culture of cells). Applying these method steps, the relative abundance of the one or more of the following protein pairs is measured and the pluripotency state of the stem cell culture is determined as shown in FIGS. 6, 7, 9-32:

a) COCH/CHGA; COCH/NID1; COCH/NPTX2; COCH/SEMA3A; FGFR1/CHGA; FGFR1/NID1; FGFR1/NPTX2; FGFR1/SEMAS3A; FST/CHGA; FST/NID1; FST/NPTX2; FST/SEMAA3; OLFML3/CHGA; OLFML/NID1; OLFML3/NPTX2; or OLFML3/SEMA3A wherein, for each pair, an increase in the relative abundance indicates a loss of pluripotency.

b) ADAMTS8/ASS1; ADAMTS8/CKB; ADAMTS8/CNMD; ADAMTS8/COCH; ADAMTS8/FGFR1; ADAMTS8/FST; ADAMTS8/OLFML3; ADAMTS8/SERPINB9 or ADAMTS8/STOM wherein, for each pair, a reduction in the relative abundance indicates a loss of pluripotency.

c) APP/ASS1; APP/CKB; APP/CNMD; APP/COCH; APP/FGFR1; APP/FST; APP/OLFML3; APP/SERPINB9 or APP/STOM wherein for each pair, a reduction in the relative abundance indicates a loss of pluripotency.

d) B3GNT7/ASS1; B3GNT7/CKB; B3GNT7/CNMD; B3GNT7/COCH; B3GNT7/FGFR1; B3GNT7/FST; B3GNT7/OLFML3; B3GNT7/SERPINB9 or B3GNT7/STOM wherein for each pair, a reduction in the relative abundance indicates a loss of pluripotency.

e) CHGA/ASS1; CHGA/CKB; CHGA/CNMD; CHGA/COCH; CHGA/FGFR1; CHGA/FST; CHGA/OLFML3; CHGA/SERPINB9; or CHGA/STOM wherein for each pair, a reduction in the relative abundance indicates a loss of pluripotency.

f) EFEMP1/ASS1; EFEMP1/CKB, EFEMP1/CNMD, EFEMP1/COCH, EFEMP1/FGFR1, EFEMP1/FST, EFEMP1/OLFML3, EFEMP1/SERPINB9 or EFEMP1/STOM wherein for each pair, a reduction in the relative abundance indicates a loss of pluripotency.

g) EPHA1/ASS1, EPHA1/CKB, EPHA1/CNMD, EPHA1/COCH, EPHA1/FGFR1, EPHA1/FST, EPHA1/OLFML3, EPHA1/SERPINB9 or EPHA1/STOM wherein for each pair, a reduction in the relative abundance indicates a loss of pluripotency.

h) EXTL2/ASS1, EXTL2/CKB, EXTL2/CNMD, EXTL2/COCH, EXTL2/FGFR1, EXTL2/FST, EXTL2/OLFML3, EXTL2/SERPINB9 or EXTL2/STOM wherein for each pair, a reduction in the relative abundance indicates a loss of pluripotency.

i) FBLN2/ASS1, FBLN2/CKB, FBLN2/CNMD, FBLN2/COCH, FBLN2/FGFR1, FBLN2/FST, FBLN2/OLFML3, FBLN2/SERPINB9 or FBLN2/STOM wherein for each pair, a reduction in the relative abundance indicates a loss of pluripotency.

j) FGF2/ASS1, FGF2/CKB, FGF2/CNMD, FGF2/COCH, FGF2/FGFR1, FGF2/FST, FGF2/OLFML3, FGF2/SERPINB9 or FGF2/STOM wherein for each pair, a reduction in the relative abundance indicates a loss of pluripotency.

k) FUCA2/ASS1, FUCA2/CKB, FUCA2/CNMD, FUCA2/COCH, FUCA2/FGFR1, FUCA2/FST, FUCA2/OLFML3, FUCA2/SERPINB9 or FUCA2/STOM wherein for each pair, a reduction in the relative abundance indicates a loss of pluripotency;

l) GALNT1/ASS1, GALNT1/CKB, GALNT1/CNMD, GALNT1/COCH, GALNT1/FGFR1, GALNT1/FST, GALNT1/OLFML3, GALNT1/SERPINB9 or GALNT1/STOM wherein for each pair, a reduction in the relative abundance indicates a loss of pluripotency.

m) GGH/ASS1, GGH/CKB, GGH/CNMD, GGH/COCH, GGH/FGFR1, GGH/FST, GGH/OLFML3, GGH/SERPINB9 or GGH/STOM wherein for each pair, a reduction in the relative abundance indicates a loss of pluripotency.

n) HEXB/ASS1, HEXB/CKB, HEXB/HEXB/CNMD, HEXB/COCH, HEXB/FGFR1, HEXB/FST, HEXB/OLFML3, HEXB/SERPINB9 or HEXB/STOM wherein for each pair, a reduction in the relative abundance indicates a loss of pluripotency.

o) IGFBP4/ASS1, IGFBP4/CKB, IGFBP4/CNMD, IGFBP4/COCH, IGFBP4/FGFR1, IGFBP4/FST, IGFBP4/OLFML3, IGFBP4/SERPINB9 or IGFBP4/STOM wherein for each pair, a reduction in the relative abundance indicates a loss of pluripotency.

p) NID1/ASS1, NID1/CKB, NID1/CNMD, NID1/COCH, NID1/FGFR1, NID1/FST, NID1/OLFML3, NID1/SERPINB9 or NID1/STOM wherein for each pair, a reduction in the relative abundance indicates a loss of pluripotency.

q) NPTX2/ASS1, NPTX2/CKB, NPTX2/CNMD, NPTX2/COCH, NPTX2/FGFR1, NPTX2/FST, NPTX2/OLFML3, NPTX2/SERPINB9 or NPTX2/STOM wherein for each pair, a reduction in the relative abundance indicates a loss of pluripotency.

r) NTS/ASS1, NTS/CKB, NTS/CNMD, NTS/COCH, NTS/FGFR1, NTS/FST, NTS/OLFML3, NTS/SERPINB9 or NTS/STOM wherein for each pair, a reduction in the relative abundance indicates a loss of pluripotency.

s) PCDHB5/ASS1, PCDHB5/CKB, PCDHB5/CNMD, PCDHB5/COCH, PCDHB5/FGFR1, PCDHB5/FST, PCDHB5/OLFML3, PCDHB5/SERPINB9 or PCDHB5/STOM wherein for each pair, a reduction in the relative abundance indicates a loss of pluripotency.

t) SEMA3A/ASS1, SEMA3A/CKB, SEMA3A/CNMD, SEMA3A/COCH, SEMA3A/FGFR1, SEMA3A/FST, SEMA3A/OLFML3, SEMA3A/SERPINB9 or SEMA3A/STOM wherein for each pair, a reduction in the relative abundance indicates a loss of pluripotency.

u) SEMA3F/ASS1, SEMA3F/CKB, SEMA3F/CNMD, SEMA3F/COCH, SEMA3F/FGFR1, SEMA3F/FST, SEMA3F/OLFML3, SEMA3F/SERPINB9 or SEMA3F/STOM wherein for each pair, a reduction in the relative abundance indicates a loss of pluripotency.

v) SFRP2/ASS1, SFRP2/CKB, SFRP2/CNMD, SFRP2/COCH, SFRP2/FGFR1, SFRP2/FST, SFRP2/OLFML3, SFRP2/SERPINB9 or SFRP2/STOM wherein for each pair, a reduction in the relative abundance indicates a loss of pluripotency.

w) TIMP4/ASS1, TIMP4/CKB, TIMP4/CNMD, TIMP4/COCH, TIMP4/FGFR1, TIMP4/FST, TIMP4/OLFML3, TIMP4/SERPINB9 or TIMP4/STOM wherein for each pair, a reduction in the relative abundance indicates a loss of pluripotency.

x) TNFRSF8/ASS1, TNFRSF8/CKB, TNFRSF8/CNMD, TNFRSF8/COCH, TNFRSF8/FGFR1, TNFRSF8/FST, TNFRSF8/OLFML3, TNFRSF8/SERPINB9 or TNFRSF8/STOM wherein for each pair, a reduction in the relative abundance indicates a loss of pluripotency.

y) WFDC2/ASS1, WFDC2/CKB, WFDC2/CNMD, WFDC2/COCH, WFDC2/FGFR1, WFDC2/FST, WFDC2/OLFML3, WFDC2/SERPINB9 or WFDC2/STOM wherein for each pair, a reduction in the relative abundance indicates a loss of pluripotency.

Reduction or increase as mentioned by reference to a reference value as explained herein.

In the different embodiments set out above, the relative protein abundance of one or more pair from a single group listed above is measured, e.g. 1, 2, 3, 4, 5, 6, 7, 8, 9, 10, 11, 12, 13, 14, 15 or 16 pairs from group a or 1, 2, 3, 4, 5, 6, 7, 8 or 9 pairs from any of groups b) to y) respectively.

In another embodiment, the method comprises measuring the relative protein abundance of one, two or more pairs selected from different groups, e.g. one, two or more pairs selected from each of groups a) to y) or one, two or more pairs selected from combinations of groups a) to y).

In one embodiment, one, two or more pairs selected from group a) are measured together with one, two or more pairs selected from selected from one or more of groups b) to y).

In one embodiment, all of the pairs listed above are measured. The method can thus be a multiplex method.

A skilled person would appreciate that the pairs above are shown with protein X (e.g. NTS) as the numerator and protein Y (e.g. E6 markers, such as ASS1) as the denominator. The invention is not limited to designating the numerator and denominator in this way and of course it is also possible to measure the relative abundance of these pairs differently by measuring protein Y/protein X.

In another aspect, the invention relates to measuring the absolute abundance of one or more protein selected from Argininosuccinate synthetase 1 (ASS1), Creatine kinase B-type (CKB), chondromodulin-1 (CNMD), Cochlin (COCH), Fibroblast Growth Factor Receptor 1 (FGFR1), Follistatin (FST), Olfactomedin-like 3 (OLFML3), Serpin B1 (SERPINB1), Stomatin (STOM), A disintegrin and metalloproteinase with thrombospondin motifs 8 (ADAMTS8), Amyloid Beta Precursor Protein (APP), UDP-GlcNAc:BetaGal Beta-1,3-N-Acetylglucosaminyltransferase 7 (B3GNT7), Chromogranin A (CHGA), EGF-containing fibulin-like extracellular matrix protein 1 (EFEMP1), Ephrin type-A receptor 1 (EPHA1), Exostosin-like 2 (EXTL2), Fibulin 2 (FBLN2), Fibroblast Growth Factor 2 (FGF2), Plasma alpha-L-fucosidase (FUCA2), Polypeptide N-acetylgalactosaminyltransferase 1 (GALNT), Gamma-Glutamyl Hydrolase (GGH), Beta-hexosaminidase subunit beta (HEXB), Insulin-like growth factor-binding protein 4 (IGFBP4), Nidogen 1 (NID1), Neuronal Pentraxin-2 (NPTX2), Neurotensin (NTS), Protocadherin beta-5 (PCDHBS), Semaphorin 3A (SEMA3A), Semaphorin 3F (SEMA3F), Secreted Frizzled Related Protein 2 (SFRP2), Metalloproteinase inhibitor 4 (TIMP4), Tumor necrosis factor receptor superfamily member 8 (TNFRSF8) and WAP four-disulfide core domain protein 2 (WFDC2) and comparing said absolute abundance to a reference value. For example, the abundance of at least 1, 2, 3, 4, 5, 6, 7, 8, 9, 10, 11,12, 13, 14, 15, 16, 17, 18, 19, 20, 21, 22, 23, 24, 25, 26, 27, 28, 29, 30, 31, 32 or 33 proteins can be measured.

In one embodiment, the absolute abundance of COCH, FGFR1, FST, OLFML3, CHGA, NID1, NPTX2 and/or SEMA3A is measured. Absolute abundance of these proteins is shown in FIG. 6. For example, absolute abundance of COCH, FGFR1, FST, OLFML3 is higher in cultures that have lost pluripotency compared to pluripotent cultures and absolute abundance of CHGA, NID1, NPTX2, SEMA3A is lower in cultures that have lost pluripotency compared to pluripotent cultures.

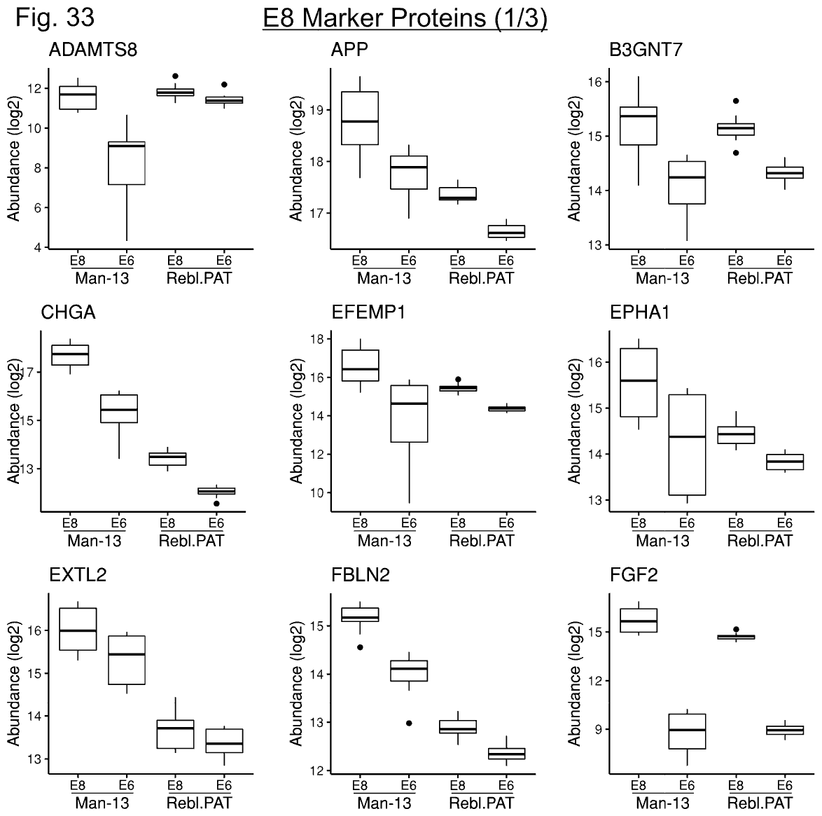

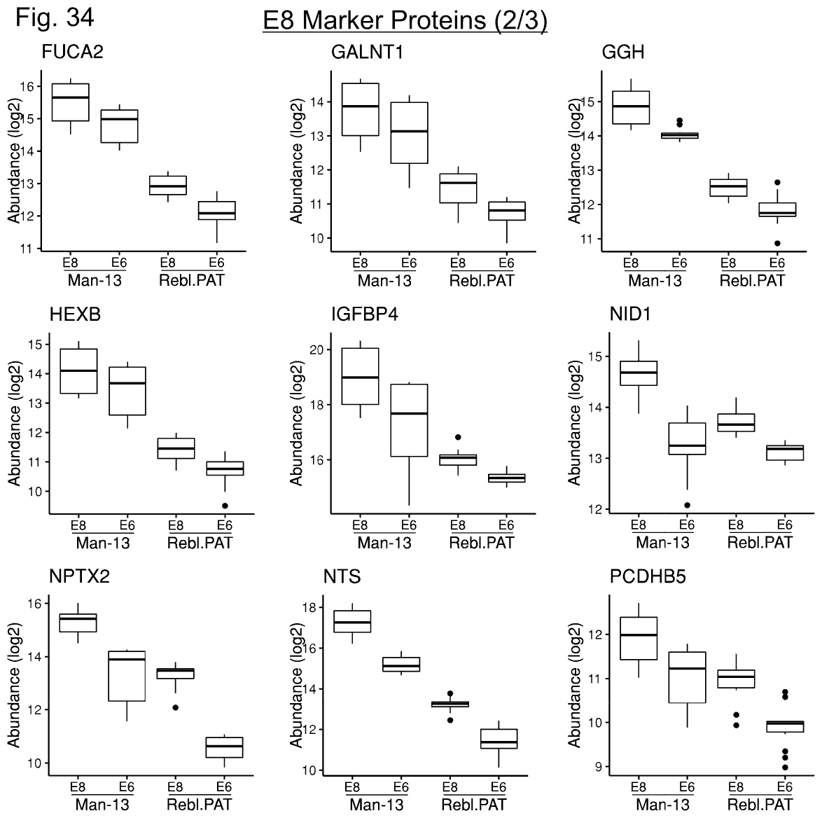

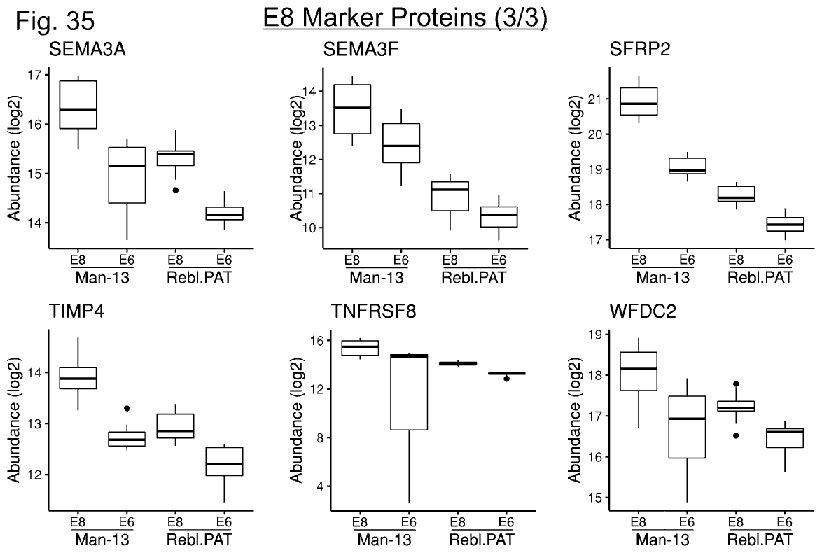

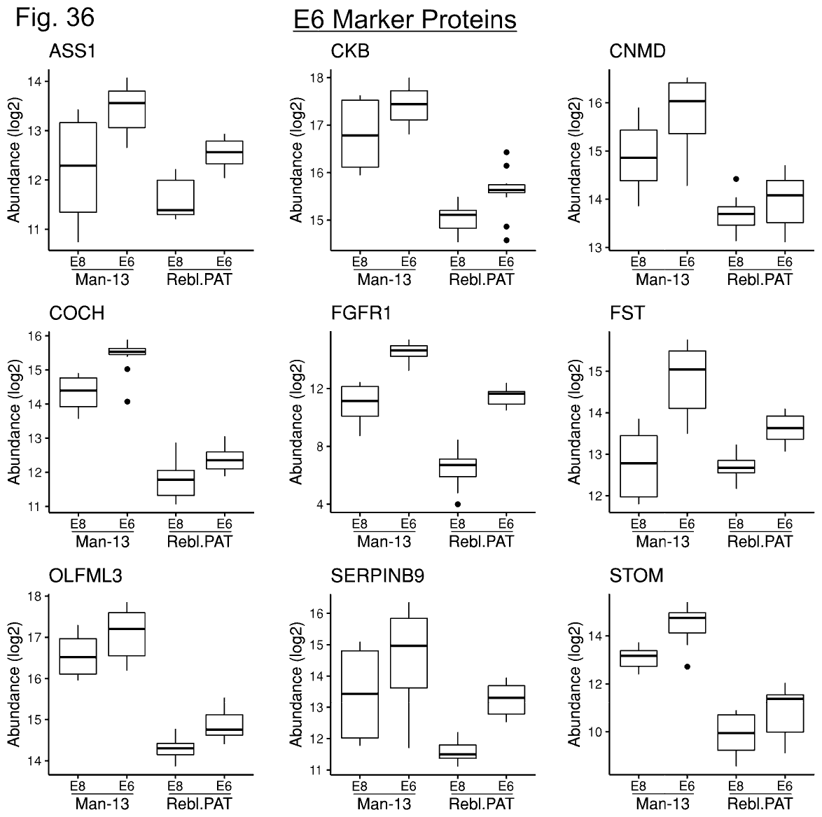

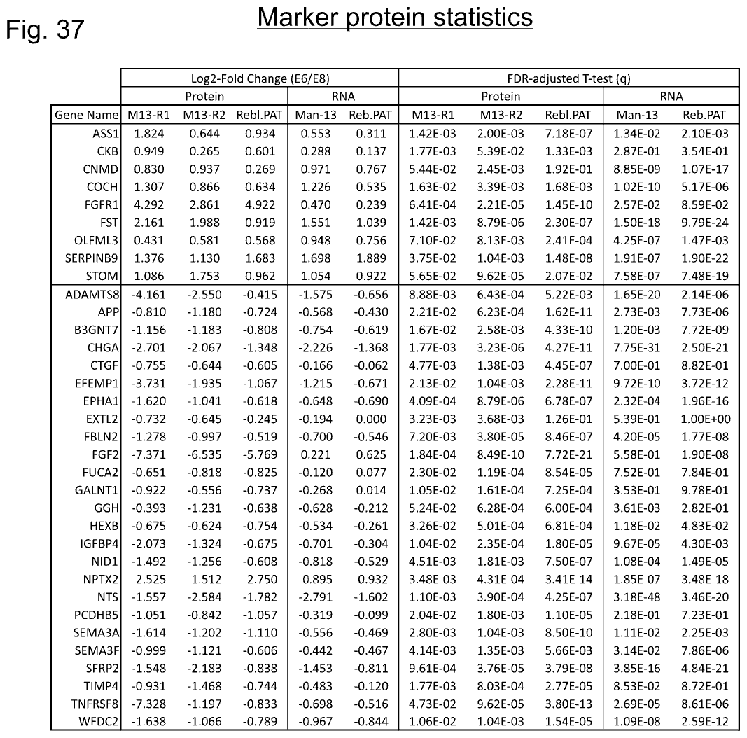

Absolute abundance of APP, ADAMTS8, B3GNT7, CHGA, EFEMP1, EPHA1, EXTL2, FBLN2, FGF2, FUCA2, GALNT1, GGH, HEXB, IGFBP4, NID1, NPTX2, NTS, PCDHBS, SEMA3A, SEMA3F, SFRP2, TIMP4, TNFRSF8 and WFDC2 is higher in cultures that are pluripotent compared to cultures that have lost pluripotency (see FIGS. 33-35). Absolute abundance of ASS1 CKB, CNMD, SERPINB9, Coch FGFr1,FSt and OLFML3 and STOM is higher in cultures that have lost pluripotency compared to pluripotent cultures (see FIG. 36).

In another embodiment, the method comprises measuring the relative protein abundance as described above and additionally measuring the absolute protein abundance of one or more of COCH, FGFR1, FST, OLFML3, CHGA, NID1, NPTX2, SEMA3A, ASS1, CKB, CNMD, SERINB9, STOM, APP, B3GNT7, EFEMP1, EPHA1 M, EXTL2, FBLN2, FGF2, FUCA2, GALNT1, GGH, HEXB, IGFBP4, NTS, PCDHBS, SEMA3A, SEMA3F, SRP2, TIMP4, TNFRSF8, WFDC2 and/or ADAMTS8. In another embodiment, the method comprises measuring the relative protein abundance as described above and additionally measuring the absolute protein abundance of one or more of COCH, FGFR1, FST, OLFML3, CHGA, NID1, NPTX2 and/or SEMA3A.

The method may comprise determining a reference value of relative abundance or absolute abundance of the test protein or protein pair. This step may form an initial step of the method so that as a first step of the method for determining the pluripotent state of a stem cell culture said method cells are cultured and the reference value is determined. Following this step, a test stem cell culture a can then be assessed. This may be the same culture as the reference culture, but which is then assessed over a time period, such as 24, 48 or 72 hours to evaluate pluripotency loss. As explained herein, the reference value may be determined in a pluripotent cell culture.

The proteins listed above are all part of the secretome of the cells, that is they are transported out of the cell or are located in the extracellular membrane. Thus, the proteins or peptide parts thereof can be measured in the culture media.

The stem cells may be in culture media and the abundance of proteins as herein above described may be the abundance of the proteins in the culture media. Advantageously, the proteins are secreted or otherwise released into the culture media by the cells and therefore spent culture media can be assessed by the present method.

The culture media may be assessed at a regular time interval, such as 12, 24, 48 and/or 72 hours or more; e.g. 4, 5, 6, 7, 8, 9, or 10 days after being seeded with stem cells. Alternatively, or additionally, the culture media may be assessed 12, 24, 48 and/or 72 hours or more; e.g. 4, 5, 6, 7, 8, 9, or 10 days after a change in growth conditions. The culture media may alternatively be assessed on a continuous basis e.g. for 12, 24, 48 and/or 72 hours or more; e.g. 4, 5, 6, 7, 8, 9, or 10 days.

It will be apparent to the skilled addressee that the abundance of the protein may be assessed by a number of means used to identify and quantify proteins. For example, the abundance of proteins may be assessed by means of Mass Spectrometry (MS). Marker protein may also be quantitated by processes involving gel electrophoresis (e.g. Western blot or two-dimensional gel electrophoresis), densitometry, fluorescence, luminescence, and/or radioactivity.

Alternatively or additionally, the abundance of the protein is assessed by means of one or more antibodies, including antibody fragments (e.g., a Fab, F(ab')2, Fv, a single chain Fv fragment (scFv) or single domain antibody, for example a VH or VHH domain) or antibody mimetic protein. It will also be apparent that a number of existing research kits could be adapted so as to assess the loss of pluripotency or potential loss of pluripotency in pluripotent cultures. For example, Enzyme-Linked ImmunoSorbent Assay (ELISA) and/or a similar technique could be employed to assess the abundance of the proteins.

In a related aspect, the method as herein above described may be for use in detecting the loss of pluripotency or potential loss of pluripotency, or the recovery of a stable pluripotent state, or indeed continuation of stable, healthy pluripotent culture growth.

In a further related aspect, the method as herein described may be for use in recovering loss of pluripotency or potential loss of pluripotency in pluripotent cultures. In this aspect, the method is used to assess the cells for the loss of pluripotency or potential loss of pluripotency and if such loss is determined, remedial agents can be provided (and/or actions taken) to the cells so as to recover them to the desired state of pluripotency, whereby pluripotency is subsequently assessed using the method as previously described. In a further related aspect, the method as herein described may be for use in detecting PSCs in a cell culture.

A method for maintaining PSCs cells in a pluripotent state, comprising causing them to express one or more of the following proteins at a higher level: APP, ADAMTS8, B3GNT7, CHGA, EFEMP1, EPHA1, EXTL2, FBLN2, FGF2FUCA2, GALNT1, GGH, HEXB, IGFBP4, NID1, NPTX2, NTS, PCDHB5, SEMA3A, SEMA3F, SFRP2, TIMP4, TNFRSF8, WFDC2 , CHGA, NID1, NPTX2, SEMA3A.

In accordance with a further aspect of the present invention, there is provided a method for growing PSCs comprising:

a) seeding a growth medium with PSCs and incubating the cells under conditions effective to enable growth;

b) testing the growth media for altered individual and/or relative abundance of one or more of the following proteins or protein pairs selected from two of the listed proteins: ASS1, CKB, CNMD, COCH, FGFR1, FST, OLFML3, SERPINB1, STOM, ADAMTS8, APP, B3GNT7, CHGA, EFEMP1, EPHA1, EXTL2, FBLN2, FGF2, FUCA2, GALNT, GGH, HEXB, IGFBP4, NID1, NPTX2, NTS, PCDHB5, SEMA3A, SEMA3F, SFRP2, TIMP4, TNFRSF8 and/or WFDC2, said altered individual and/or relative abundance is indicative of the loss of pluripotency or potential loss of pluripotency of the cells.

In a related aspect of the invention, the cells are recovered from the loss of pluripotency or potential loss of pluripotency by adding one or more agents to arrest loss of pluripotency or taking remedial action to arrest loss of pluripotency.

Pluripotency may be recovered by subculture of the cells, and/or replacement of culture medium with fresh medium, and/or the use of one or more agents to arrest or reverse loss of pluripotency.

In accordance with a further aspect of the present invention, there is provided a kit of parts for assessing the pluripotent state of pluripotent stem cell cultures, the kit comprising:

a) one or more components which can bind to or otherwise detect one or more of the following proteins: ASS1, CKB, CNMD, COCH, FGFR1, FST, OLFML3, SERPINB1, STOM, ADAMTS8, APP, B3GNT7, CHGA, EFEMP1, EPHA1, EXTL2, FBLN2, FGF2, FUCA2, GALNT, GGH, HEXB, IGFBP4, NID1, NPTX2, NTS, PCDHB5, SEMA3A, SEMA3F, SFRP2, TIMP4, TNFRSF8 and/or WFDC2; and optionally

b) means for quantifying binding and/or detection of the one or more components with the one or more proteins, such as an antibody or antibody fragment.

The kit may comprise a control representing the quantitative value of the proteins or protein abundance expected in cell media when cells are in a pluripotent state and may enable users to establish base-line values of pluripotent cells in their own culture system to increase the applicability and accuracy of the method.

The means for detecting quantitative binding of the one or more components with the one or more proteins may further indicate whether the one or more proteins are elevated or reduced in a sample.

The kit may comprise one, two or more components which can bind to on or two or more, e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33 separate proteins and the means for detecting quantitative binding detects the quantitative binding of the two or more components with the two separate proteins. Preferably, the kit will comprise a first component which can bind to a first protein selected from: ASS1, CKB, CNMD, COCH, FGFR1, FST, OLFML3, SERPINB1, STOM; and a second component which can bind to a second protein selected from: ADAMTS8, APP, B3GNT7, CHGA, EFEMP1, EPHA1, EXTL2, FBLN2, FGF2, FUCA2, GALNT, GGH, HEXB, IGFBP4, NID1, NPTX2, NTS, PCDHB5, SEMA3A, SEMA3F, SFRP2, TIMP4, TNFRSF8 and/or WFDC2.

The means for detecting quantitative binding may indicate the relative quantity of each of the two or more separate proteins so as to enable the individual and/or relative abundance of the two or more proteins to be calculated.

The kit may further comprise one or more reaction vessels so as to enable the components to be mixed with spent or conditioned growth media (or fractions thereof).

The one or more components may comprise an antibody or antibody fragment thereof or similar. The kit may comprise an Enzyme-Linked ImmunoSorbent Assay (ELISA) and/or similar technique. The kit may comprise components suitable for use in detecting proteins using Mass Spectrometry. In an aspect, the kit may be for use in the assessing the pluripotent state of pluripotent stem cell cultures in the spent or conditioned growth media of the cells.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification, including reference to sequence database identifiers, are incorporated herein by reference in their entirety. Unless otherwise specified, when reference to sequence database identifiers is made, the version number is 1. “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

The invention further relates to the following embodiments.

1. A method for assessing the pluripotent state of pluripotent stem cell cultures by determining the individual abundance, and/or relative abundance of one or more of the following proteins: Argininosuccinate synthetase 1 (ASS1), Creatine kinase B-type (CKB), chondromodulin-1 (CNMD), Cochlin (COCH), Fibroblast Growth Factor Receptor 1 (FGFR1), Follistatin (FST), Olfactomedin-like 3 (OLFML3), Serpin B1 (SERPINB1), Stomatin (STOM), A disintegrin and metalloproteinase with thrombospondin motifs (ADAMTS8), Amyloid Beta Precursor Protein (APP), UDP-GlcNAc:BetaGal Beta-1,3-N-Acetylglucosaminyltransferase 7 (B3GNT7), Chromogranin A (CHGA), EGF-containing fibulin-like extracellular matrix protein 1 (EFEMP1), Ephrin type-A receptor 1 (EPHA1), Exostosin-like 2 (EXTL2), Fibulin 2 (FBLN2), Fibroblast Growth Factor 2 (FGF2), Plasma alpha-L-fucosidase (FUCA2), Polypeptide N-acetylgalactosaminyltransferase 1 (GALNT), Gamma-Glutamyl Hydrolase (GGH), Beta-hexosaminidase subunit beta (HEXB), Insulin-like growth factor-binding protein 4 (IGFBP4), Nidogen 1 (NID1), Neuronal Pentraxin-2 (NPTX2), Neurotensin (NTS), Protocadherin beta-5 (PCDHB5), Semaphorin 3A (SEMA3A), Semaphorin 3F (SEMA3F), Secreted Frizzled Related Protein 2 (SFRP2), Metalloproteinase inhibitor 4 (TIMP4), Tumor necrosis factor receptor superfamily member 8 (TNFRSF8) and/or WAP four-disulfide core domain protein 2 (WFDC2).

2. The method of embodiment 1, wherein pluripotency is assessed by determining the individual abundance and/or relative abundance of one or more of the following proteins: Cochlin (COCH), Fibroblast Growth Factor Receptor 1 (FGFR1), Follistatin (FST), Olfactomedin-like3 (OLFML3), Chromogranin A (CHGA), Nidogen 1 (NID1), Neuronal Pentraxin-2 (NPTX2), and/or Semaphorin 3A (SEMA3A).

3. The method of embodiments 1 or 2, wherein the method comprises comparing the individual abundance and/or relative abundance of the one or more proteins to a known or baseline individual abundance and/or relative abundance of the one or more proteins.

4. The method of any preceding of embodiment wherein an elevated or reduced individual abundance and/or relative abundance of the one or more proteins indicates the loss of pluripotency or potential loss of pluripotency.

5. The method of any preceding of embodiment, wherein the method comprises determining a baseline individual abundance and/or relative abundance.

6. The method of embodiment 5, wherein the baseline individual abundance and/or the relative abundance may be determined by assessing the individual and/or relative protein abundance and/or secretome of cells that demonstrate pluripotency.

7. The method of embodiments 5 or 6, wherein a deviation of individual abundance and/or relative abundance from the baseline can indicate a loss of pluripotency or potential loss of pluripotency.

8. The method of any preceding of embodiment, wherein the method comprises determining the individual abundance and/or relative abundance between two or more of the proteins.

9. The method of embodiment 8, wherein the method comprises determining the individual and/or relative abundance between the two or more proteins, wherein a first protein is selected from a first set of proteins comprising: Argininosuccinate synthetase 1 (ASS1), Creatine kinase B-type (CKB), chondromodulin-1 (CNMD), Cochlin (COCH), Fibroblast Growth Factor Receptor 1 (FGFR1), Follistatin (FST), Olfactomedin-like 3 (OLFML3), Serpin B1 (SERPINB1), Stomatin (STOM); and a second protein is selected from a second set of proteins comprising: A disintegrin and metalloproteinase with thrombospondin motifs 8 (ADAMTS8), Amyloid Beta Precursor Protein (APP), UDP-GlcNAc:BetaGal Beta- 1,3-N-Acetylglucosaminyltransferase 7 (B3GNT7), Chromogranin A (CHGA), EGF-containing fibulin-like extracellular matrix protein 1 (EFEMP1), Ephrin type-A receptor 1 (EPHA1), Exostosin-like 2 (EXTL2), Fibulin 2 (FBLN2), Fibroblast Growth Factor 2 (FGF2), Plasma alpha-L-fucosidase (FUCA2), Polypeptide N-acetylgalactosaminyltransferase 1 (GALNT), Gamma-Glutamyl Hydrolase (GGH), Beta-hexosaminidase subunit beta (HEXB), Insulin-like growth factor-binding protein 4 (IGFBP4), Nidogen 1 (NID1), Neuronal Pentraxin-2 (NPTX2), Neurotensin (NTS), Protocadherin beta-5 (PCDHB5), Semaphorin 3A (SEMA3A), Semaphorin 3F (SEMA3F), Secreted Frizzled Related Protein 2 (SFRP2), Metalloproteinase inhibitor 4 (TIMP4), Tumor necrosis factor receptor superfamily member 8 (TNFRSF8) and/or WAP four-disulfide core domain protein 2 (WFDC2).

10. The method of embodiment 9, wherein preferably, the first protein is selected from a first set of proteins comprising: Cochlin (COCH), Fibroblast Growth Factor Receptor 1 (FGFR1), Follistatin (FST) and Olfactomedin-like3 (OLFML3); and a second protein is selected from a second set of proteins comprising: Chromogranin A (CHGA), Nidogen 1 (NID-1), Neuronal Pentraxin-2 (NPTX2), and/or Semaphorin 3A (SEMA3A).

11. The method of any preceding of embodiments, wherein the individual protein abundance and/or the relative protein abundance may indicate the loss of pluripotency or potential loss of pluripotency, and/or the recovery of pluripotency.

12. The method of embodiments 8 to 11, wherein a baseline relative abundance between the two or more proteins is determined.

13. The method of embodiment 12, wherein the baseline relative abundance between the two or more proteins may be determined by assessing the individual relative protein abundance and/or secretome of cells that demonstrate pluripotency.

14. The method of embodiments 12 or 13, wherein a deviation from the baseline indicates the loss of pluripotency or potential loss of pluripotency.

15. The method of any preceding of embodiment wherein individual and/or relative protein abundance indicates the loss of pluripotency and/or potential loss of pluripotency and/or the recovery of pluripotency.

16. The method of any preceding of embodiment, wherein the pluripotent stem cells are in culture media.

17. The method of any preceding of embodiment, wherein the abundance of proteins are the abundance of the proteins in the culture media.

18. The method of any preceding of embodiment, wherein the culture media is assessed 72 hours after being seeded with pluripotent stem cells.

19. The method of any preceding embodiment, wherein the culture media is assessed at a regular time interval or on a continuous basis.

20. The method of any preceding of embodiment, wherein the abundance of the proteins are assessed by means of Mass Spectrometry (MS).

21. The method of any preceding of embodiment, wherein the abundance of the proteins are assessed by means of one or more antibodies.

22. The method of embodiment 21, wherein the abundance of the proteins are assessed by means of an Enzyme-Linked ImmunoSorbent Assay (ELISA) and/or similar technique.

23. The method of any preceding of embodiment, for use in detecting the loss of pluripotency or potential loss of pluripotency and/or recovery of stable pluripotent state.

24. The method of any of embodiments 1 to 22, for use in recovering loss of pluripotency or potential loss of pluripotency in pluripotent stem cells, whereby the method is used to assess the cells for the loss of pluripotency or potential loss of pluripotency and if such loss is determined remedial agents can be provided to the cells, and/or remedial actions taken, so as to recover them to the desired state of pluripotency, whereby pluripotency is subsequently assessed using the method of embodiments 1 to 22.

25. A method for growing pluripotent stem cells comprising:

a) seeding a growth medium with pluripotent stem cells and incubating the cells under conditions effective to enable growth;

b) testing the growth media for altered individual and/or relative abundance of one or more of the following proteins: Argininosuccinate synthetase 1 (ASS1), Creatine kinase B-type (CKB), chondromodulin-1 (CNMD), Cochlin (COCH), Fibroblast Growth Factor Receptor 1 (FGFR1), Follistatin (FST), Olfactomedin-like 3 (OLFML3), Serpin B1 (SERPINB1), Stomatin (STOM), A disintegrin and metalloproteinase with thrombospondin motifs 8 (ADAMTS8), Amyloid Beta Precursor Protein (APP), UDP-GlcNAc:BetaGal Beta- 1,3-N-Acetylglucosaminyltransferase 7 (B3GNT7), Chromogranin A (CHGA), EGF-containing fibulin-like extracellular matrix protein 1 (EFEMP1), Ephrin type-A receptor 1 (EPHA1), Exostosin-like 2 (EXTL2), Fibulin 2 (FBLN2), Fibroblast Growth Factor 2 (FGF2), Plasma alpha-L-fucosidase (FUCA2), Polypeptide N-acetylgalactosaminyltransferase 1 (GALNT), Gamma-Glutamyl Hydrolase (GGH), Beta-hexosaminidase subunit beta (HEXB), Insulin-like growth factor-binding protein 4 (IGFBP4), Nidogen 1 (NID1), Neuronal Pentraxin-2 (NPTX2), Neurotensin (NTS), Protocadherin beta-5 (PCDHBS), Semaphorin 3A (SEMA3A), Semaphorin 3F (SEMA3F), Secreted Frizzled Related Protein 2 (SFRP2), Metalloproteinase inhibitor 4 (TIMP4), Tumor necrosis factor receptor superfamily member 8 (TNFRSF8) and/or WAP four-disulfide core domain protein 2 (WFDC2), said altered individual and/or relative abundance being indicative of the loss of pluripotency or potential loss of pluripotency of the cells.

26. The method of embodiment 25, wherein the method comprises comparing the individual abundance and/or relative abundance of the one or more proteins to a pre-determined baseline or establishing said baseline.

27. The method of embodiment 25 or 26, wherein the cells are recovered from the loss of pluripotency or potential loss of pluripotency by adding one or more agents to arrest loss of pluripotency or taking remedial action to arrest loss of pluripotency.

28. The method of any of embodiments 25 to 27, wherein pluripotency may be recovered by subculture of the cells, and/or replacement of culture medium with fresh medium, and/or the use of one or more agents to arrest loss of pluripotency.

The invention is further described in the following non-limiting examples.

### EXAMPLES

With a view to developing a non-invasive method for monitoring the healthy pluripotent state of human stem cells in culture, the inventors undertook proteomic analysis of the spent medium from cultured embryonic (Man-13) and induced (Rebl.PAT) pluripotent stem cells. Cells were grown in E8 medium to maintain pluripotency, and then transferred to E6 medium - identical but lacking the growth factors FGF2 and TGFβ—for 48 hours to replicate an early, undirected dissolution of pluripotency. Conditioned medium was harvested and analysed by LC-MS/MS after 48 hours of growth-factor starvation. The inventors identified a distinct proteomic footprint associated with early loss of pluripotency in both cell lines, and a strong correlation with changes in the transcriptome. Several high confidence protein biomarkers were identified which demonstrated consistent correlation with the pluripotent state, such that they may be utilised as a rapid diagnostic for pluripotency loss. Additionally, we sought to overcome the absence of a functional normalisation method for secreted protein quantification by developing a novel paired-protein ratio metric. We hypothesised that proteins which increased or decreased in response to the same stimulus would change more consistently and detectibly in relation to each other, than to other normalisation standards. We identified 4 E8-enriched and 4 E6-enriched proteins which were demonstrated to give 16 protein-pair ratios that consistently, robustly distinguished the two cell states in both cell lines, and confirmed this in 3 further lines by Western blotting. We further demonstrated that returning the, 48 hour, E6-cultured cells to E8 for 7 days recovered healthy levels of pluripotency and that the ratios of our pluripotency associated markers in the secretome reflected this. Further work to validate the prospective use of these protein markers will provide a rapid quantitative readout of pluripotent stem cell state and early warning of culture quality degradation at a stage when this is recoverable without sacrifice of stem cell product.

Although the clinical potential of regenerative medicine, including approaches using human pluripotent stem cells (hPSCs), remains unrivalled, major hurdles exist in translating promising treatments into scalable, reproducible commercial processes. In particular, maintaining hPSCs in their pluripotent state has proven challenging and labour intensive. Whilst advances have been made in automating the culture and expansion of high quality PSCs, such as by the development of the SelecT (TAP biosystems—Hertfordshire, UK), Freedom EVO (TECAN Trading AG, Switzerland) systems, or technologies developed by Tokyo Electron (Kyoto, Japan); an issue which remains to be overcome in PSC culture of all scales is the absence of a rapid, reproducible, non-invasive and quantitative metric for pluripotent cultures. The most commonly used techniques all have major drawbacks when it comes to their use in PSC manufacture. For instance, using immunofluorescence for assessing the core pluripotency transcription factors and cell surface markers does not accurately pick up initial loss of pluripotency in entire cultures, but rather relatively late loss of pluripotency in individual cells. Immunofluorescence is also imprecise, non- quantitative, cannot be integrated into the manufacturing process, and the tested cells are lost form the culture. Using flow cytometry for identifying loss of pluripotency markers shows profound run variability and also sacrifices cell product. Currently the most quantifiable metrics of pluripotency available are RNA-based assays on cells e.g. Pluri-test [3] and ScoreCard [4], or teratoma assays [5]. However, these metrics are not currently cost-effective, require sacrifice of cell product, and take too long to provide results on at- risk cultures which have likely been permanently compromised by the time the results are available. The most rapid and cost-effective tool researchers currently have is analysis of cell morphology, however this is inherently subjective. While attempts have been made to automate and quantify the characteristics of PSC morphology [6, 7], it remains a low-resolution way in which to measure pluripotency [8]. There are thus no convenient quantitative assays which can pick up very early loss of pluripotency.

To address these limitations the inventors sought to identify secreted protein biomarkers which could be identified in spent culture media within 48 hours of the onset of potential pluripotency loss. Although the secretome of hPSCs has been studied in the past [9-13], there is yet to be a comparative study aiming to identify proteins in media indicative of the pluripotent state, and it's loss. It is well established that hPSCs rely on the signalling pathways down stream of FGF-2 and TGFβ family members for their self-renewal [14- 16]. After removal of these growth factors from standard hPSC culture medium the cells lose the robust regulatory pluripotency network, a prerequisite for lineage differentiation [17]. Therefore, the inventors used the removal of FGF-2 and TGFβ from the medium to replicate culture deterioration, aiming to identify protein biomarkers which could be detected in spent medium while it is still possible to rescue cell product. Using LC-MS/MS of conditioned medium, we identified 4 E8- and 4 E6-enriched proteins in the secretome which, are highly indicative of healthy or unstable pluripotent cultures. Additionally, the inventors demonstrate that by multiplexing proteins enriched in E8 against those enriched in E6, we are able to increase the scale and robustness of the change between conditions in a manner which could lead to the development of a highly sensitive assay for very early signs of pluripotency dissolution and culture deterioration. the inventors further demonstrate the utility of these markers by returning E6 cultures to E8 medium, leading to a full recovery of pluripotency as determined both by our marker proteins, and teratoma assays. Such indicators in the spent medium will be particularly useful in a scale up culture setting in order to reverse culture deterioration while avoiding cell sampling and loss of product and are amenable to simple detection assays.

### Results

Pluripotency loss after 48 hours of FGF2/TGFβ removal is at the limits of detection by flow cytometry and immunocytochemistry (ICC).

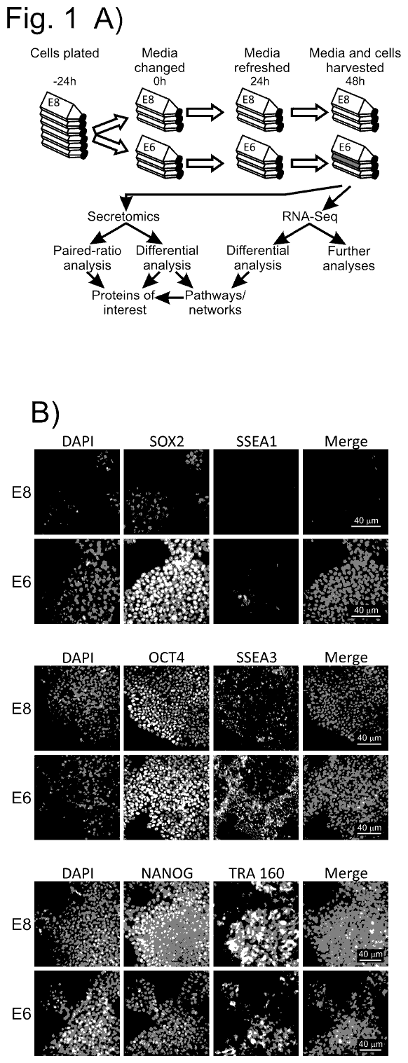

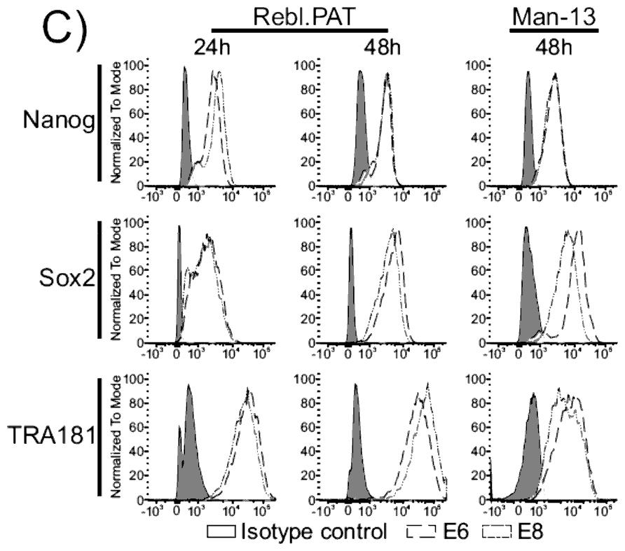

HESC (Man-13: [18]) and iPSC (Rebl.PAT: [19]) lines (collectively referred to as PSCs) were cultured in either pluripotency maintenance medium (E8 [20]), or identical medium lacking the pluripotency maintenance factors FGF2 and TGFβ1 (E6 [21]) for 48 hours, before analysis (FIG. 1.A). A previous study indicated that under these conditions, lineage specific marker protein mRNA is not definitively expressed until at least four days, while markers of pluripotency loss are detectable by RNA microarray after as little as 48 hours of FGF2/TGFβ loss [17]. Therefore, it was hypothesised that it should be possible to identify secretome changes associated with the onset of dissolution of pluripotency at this early time point, enabling cultures to be identified at a point at which they can be rescued.

After 48 hours, immunocytochemistry (ICC) indicated that cells in both E8 and E6 exhibited broadly comparable levels of pluripotency markers OCT4 and SSEA3 with a slight decrease in NANOG and TRA160 in E6 (FIG. 1B). SSEA1 (an early differentiation marker) was observed at slightly higher levels in E6 (FIG. 1B). Similar expression patterns were observed by flow cytometry, after both 24 and 48 hours of culture in E6, with pluripotency-associated markers NANOG and TRA1-81 very marginally decreased in E6 (FIG. 1C).

By both ICC and flow cytometry, Sox2 was observed to be moderately increased after 48 hours of FGF2/TGFβ loss. Although it is a pluripotency-associated marker, Sox2 expression increases upon SMAD2/3 inhibition and FGF2 deprivation [22]. Increases in Sox2 expression in PSCs can initiate differentiation [23, 24] and Sox2 has an important role in specification and differentiation of PSCs towards the neurectoderm lineage [25-27] (FIG. 1.B, C). Finally, when qPCR was performed on RNA from these cells, Oct4 and Nanog did not demonstrate significant changes, and Sox2 was consistently significantly upregulated. Additionally, no significant change in viability of cells between conditions was observed either by Apotox-Glo assay [28] or NucleoCounter NC-200 Via-1 cassettes (data not shown).

These data indicate that whilst current routine metrics can identify some subtle changes in pluripotency-associated markers after 48 hours of growth factor withdrawal, these are only distinguishable when comparing these samples with high quality pluripotent controls and would be almost impossible to identify in isolation as indicative of degradation of stem cell culture quality.

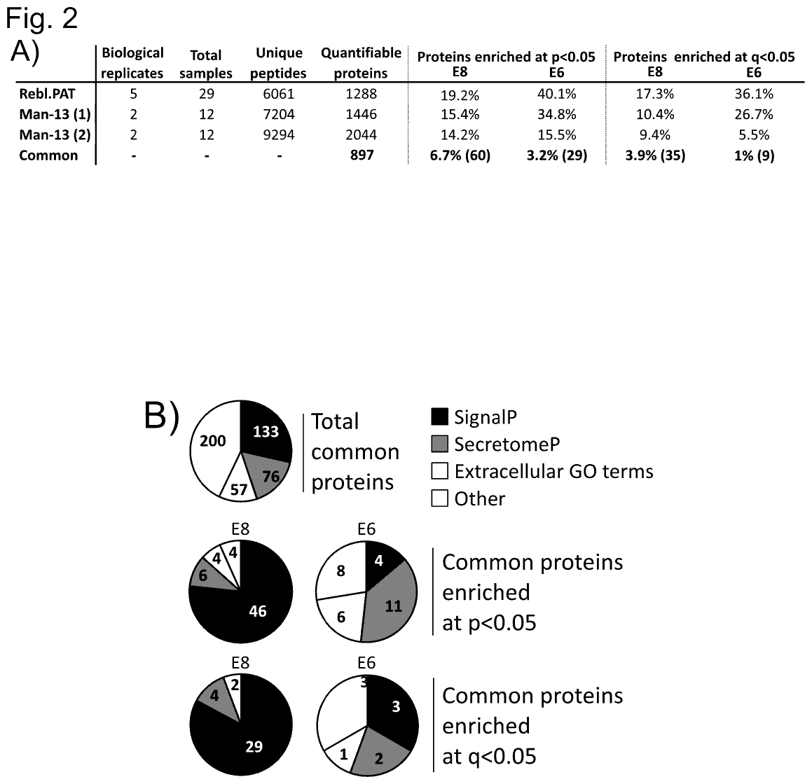

Consistent, detectible changes in the secretome are observable after 48 hours of FGF2/TGFβ removalMan-13 and Rebl.PAT cells were cultured in Vitronectin-N coated T75 flasks in E8 medium for 3 passages before being plated for proteomic analysis. The medium was removed, replaced, and collected, then the protein prepared and enriched for LC-MS/MS as described in FIG. 1A and Materials and Methods. 5 biological by three technical replicates of Rebl.PAT cells were performed, and all analysed together by label-free mass-spec (FIG. 2A). Man-13 samples were analysed by LC-MS/MS in two batches of two biological by three technical replicates to ensure that any identified biomarkers would be resilient to run variation (FIG. 2A). A subset of the cells at the 48 hour time-point were also collected and processed for RNA-Seq analysis.

In terms of general data trends between conditions, a smaller number of proteins were enriched (p<0.05) in E8 media compared to E6, and these proteins demonstrated a higher fold-change, and were more consistent between cell lines and experimental replicates than E6-enriched proteins (FIG. 2A). The distribution of the protein enrichment was asymmetrical between conditions, with more proteins enriched in E6 than E8, however E6-enrichment compared to E8 was smaller in magnitude and less consistent between samples, concomitant with an increased proportion of intracellular proteins (FIG. 2B).

E8-enriched proteins were primarily classically-secreted, as identified by the presence of an amino-acid secretion tag (SignalP), an amino-acid sequence indicative of non-classical secretion (SecretomeP), or by annotation with the cellular component gene ontology (GO) tags; extracellular space, extracellular vesicle, cell surface, or extracellular region (FIG. 2B). By contrast fewer E6 enriched (p<0.05) proteins were identified as classically secreted by the above metrics. However, classically secreted E6-enriched proteins exhibited a higher level of statistical consistency than other p<0.05 enriched proteins, suggesting that these changes may be more biologically rooted than the more stochastic enrichment of intracellular proteins in the secretome which may be caused by a small increase in cell lysis or leakage.

Finally, the secretome was analysed for the GO terms of significantly enriched proteins (p<0.05) across both cell lines. These identified the ‘Extracellular space’ category as the most common for E8 enriched proteins, whereas the same category was much lower in E6, in which the ‘membrane’ and ‘extracellular matrix’ categories ranked much higher (FIG. 2C).

### Correlation Between Changes in Protein and RNA Abundance Between Conditions

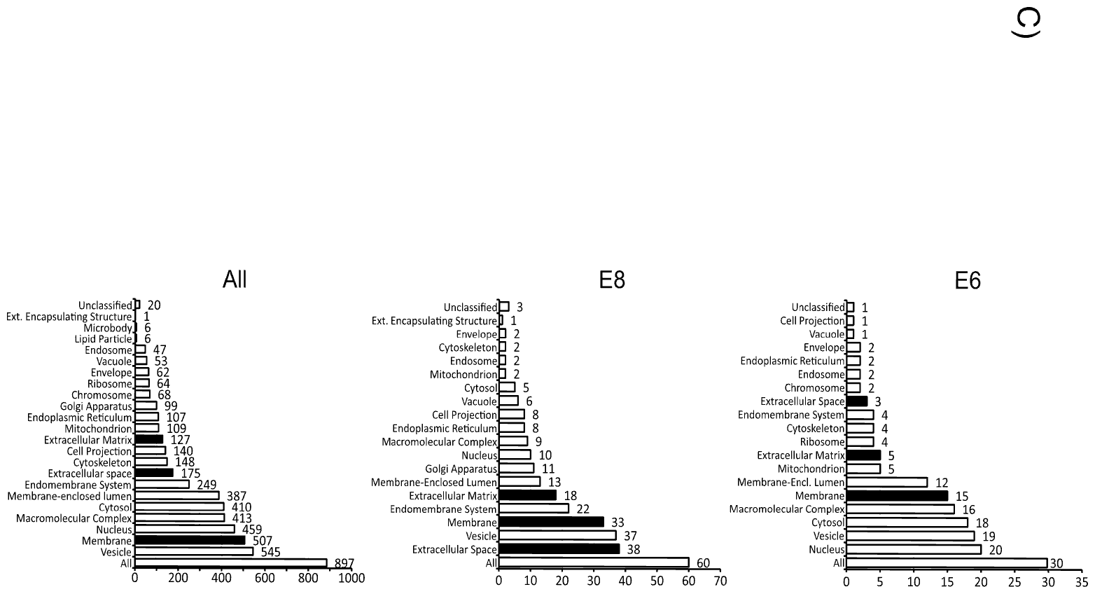

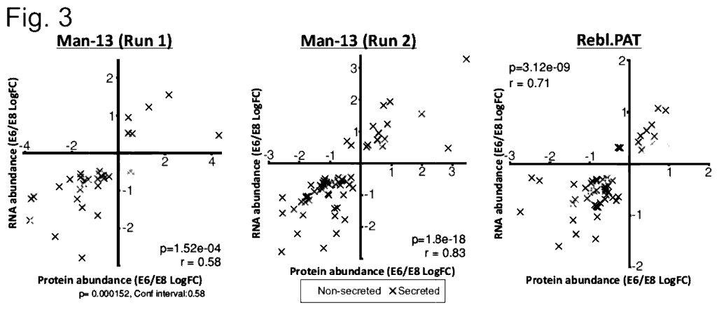

To identify whether the observed proteomic changes were indicative of fundamental changes in the pluripotent state of cells, rather than simply a transient effect restricted to the immediate downstream signalling environment of FGF2/TGFβ1 receptors, significant changes in the secretome (q<0.05) were compared against significant changes (q<0.05) in the transcriptome of the same cells (FIG. 3). The direction of significant changes between E8 and E6 cells demonstrated strong protein/RNA concomitance (M13R1 r=0.85; M13R2 r=0.78, Rebl.PAT r=0.66). Further, where disagreements in RNA/Protein enrichment occurred, these proteins were commonly intracellular, rather than secreted.

### Two-Proteins Ratio Markers

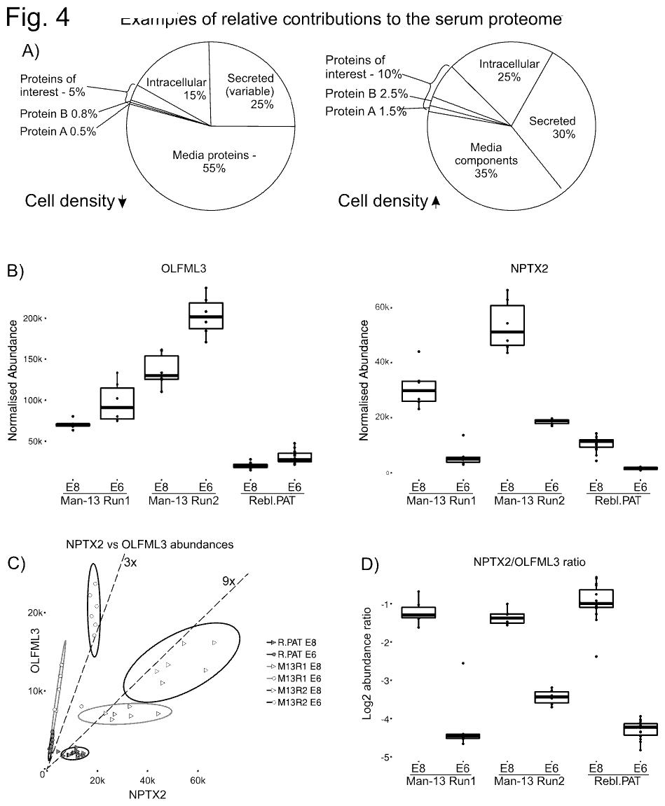

As E8 and E6 media both have high concentrations of transferrin, normalising the secreted proteome of stem cells against total protein content in the media is likely to introduce statistical uncertainty and error. Slight variabilities in the relative contributions of cells and media to the total protein content of conditioned media caused by differences in density, viability, or growth rate could drastically change the normalised abundance of secreted proteins despite consistent secretion by cells (FIG. 4A). To bypass this issue a novel analysis strategy was developed to identify proteins which were changing in response to the condition of interest. Instead of comparing all proteins to a single normalisation factor, identification of multiple pairs of proteins was sought. The relative abundances of these could be used to discriminate accurately between conditions. Identifying these protein pair ratios within each individual sample removes the issue of variable total protein abundance, as both proteins will be affected identically by cell density or similar cell variation. This is demonstrated in FIG. 4, a script was written in R which calculated the pair-wise ratio of abundance between every combination of proteins to identify those which were discriminatory between conditions. The ability of these pairs to differentiate between conditions was determined by their reaching an FDR-adjusted p-value of q<0.05. Proteins passing this threshold were identified and expressed as a network, with each protein as nodes and paired ratios as edges (FIG. 5A). The most interconnected proteins were selected for more rigorous analysis (FIG. 5B-C).

### Confirmation of Marker Protein-Pairs by Western Blotting

Eight proteins were selected for Western blot based upon their success across all analysis methodologies, their biological relevance, and the corresponding changes in their RNA levels (FIG. 5C). Additionally, it was demonstrated that in every case, relative abundances of the selected proteins provided an improved statistical difference between conditions, compared with each protein's abundance individually (data not shown). Selecting four proteins enriched in each condition, resulted in 16 ratio pairs which are all diagnostic for pluripotency loss across all samples in both cell lines (FIG. 6). Of these proteins; Cochlin (COCH), FGF Receptor-1 (FGFR1), Follistatin (FST) and Olfactomedin-like 3 (OLFML3) increased in E6 compared with E8, and Chromogranin A (CHGA), Nidogen 1 (NID1), Neuronal Pentraxin-2 (NPTX2) and Semaphorin 3A (SEMA3A) increased in E8 compared with E6. It was noteworthy that data from both Man-13 LC-MS/MS runs were successfully pooled in this analysis, confirming that these marker ratios are consistent not only across both technical and biological replicates, but also different label-free LC-MS/MS experiments, demonstrating the marker protein's resistance to run variance.

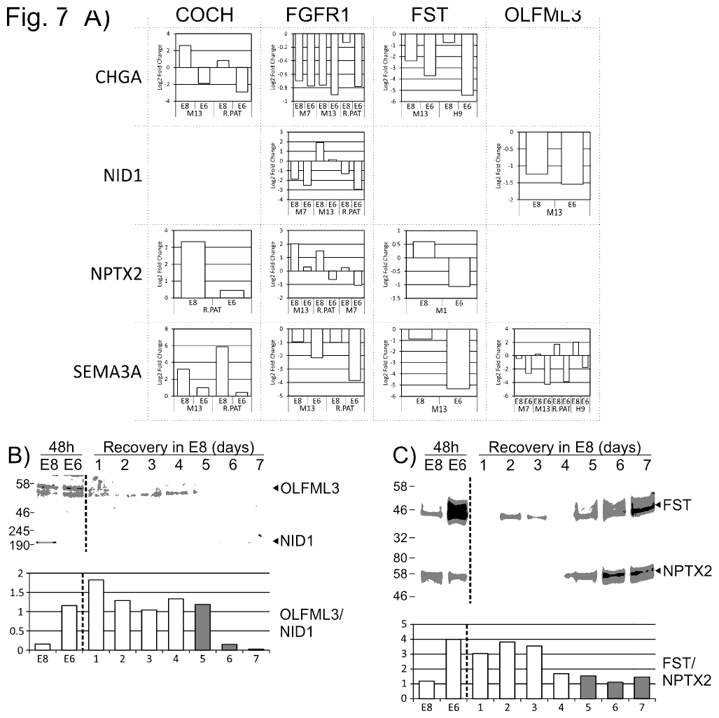

Based upon the strength of these markers in the LC-MS/MS data, conditioned media samples (collected as described in FIG. 1A) from the hESC lines Man-1, Man-7, Man-13, H9 and the iPSC line Rebl.PAT were probed by Western blot for the relative abundances of these marker proteins (FIG. 7). Each protein- pair shown was probed for, on the same membrane, and the abundance ratios calculated by densitometry in ImageJ. These Western blots confirmed that the changes observed by LC-MS/MS are highly robust and discriminatory across cells from different genetic backgrounds. The consistency of the ratio changes across multiple lines and between different combinations of proteins confirms the generalisability of this methodology as a discriminatory metric for incipient pluripotency loss across PSC lines.

### Recovery of Protein-Pair Ratio Abundances After 48 Hours in E6 Medium

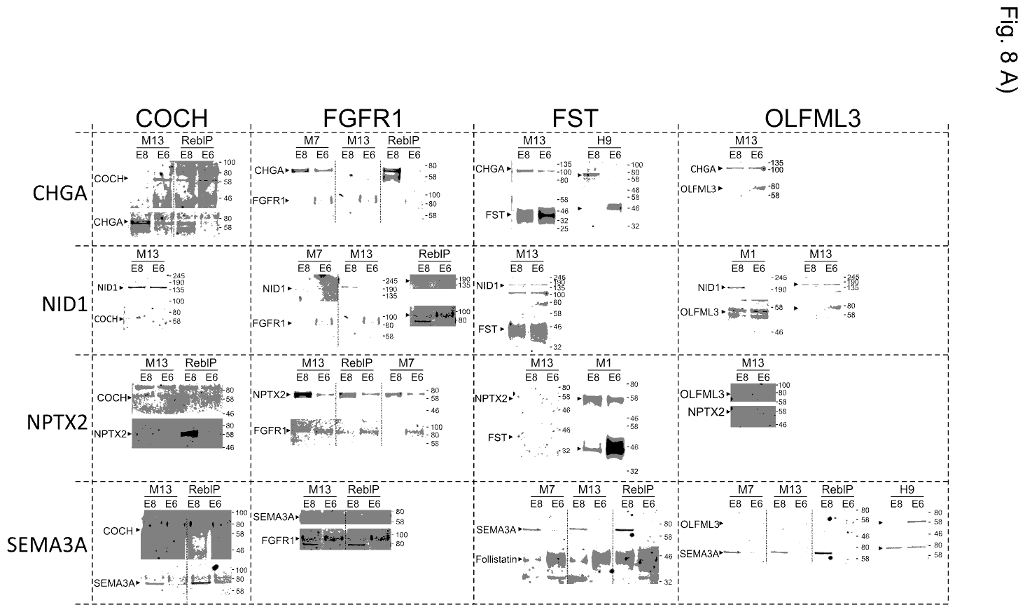

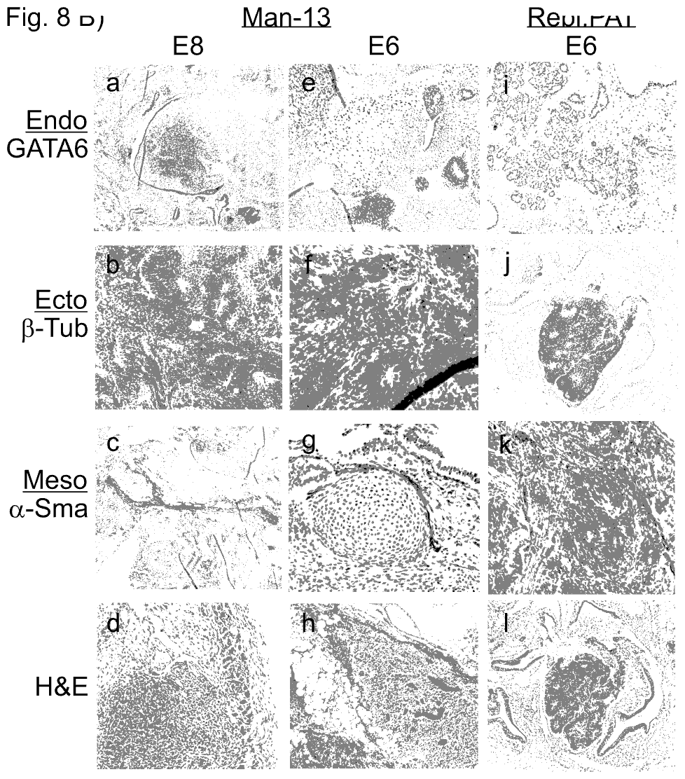

An important component of an assay for imminent loss of pluripotency and decreased PSC culture quality is the ability to detect a degradation of culture quality while the cells are recoverable. We therefore repeated the experiments indicated above but this time replaced some cells in E8 medium for 7 days after the 2 days in E6. Using Man-1 cells we demonstrated that the ratio between two pairs of the protein biomarkers selected from our analysis (OLFML3-NID1 and FST-NPTX2) returned to their pluripotent levels after 7 days of E8 recovery (FIG. 8 A, B). Moreover cells cultured for 48 hours in E6 and recovered for 7 days in E8 media can still form teratomas (FIG. 8B) demonstrating differentiation to tissues of all three germ layers. This suggests not only that the pluripotency of these cells can be fully recovered after 48 h in E6, but also that our marker proteins can robustly track this recovery. This confirms that the changes we observe are not simply molecular markers of the immediate loss of growth factor: the proteins take several days to return to their baseline, rather than returning immediately after the FGF2 and TGFβ1 are restored. Thus at the time pluripotency loss is recognised by these early marker changes, cultures can still be recovered.

### Discussion

In this study we identified a series of proteins which function as secreted, early marker proteins for degradation of a pluripotent stem cell culture. Of this long-list of marker proteins (summarised in the FIG. 5B network) a shortlist of eight proteins was identified for further analysis by Western blot. These proteome changes were also reflected by changes in gene transcription.

### Biological Relevance of Marker Proteins

Although the proteomes of both hESC and iPSC lines have been investigated by a variety of mass spectrometry techniques in the past [9-12] the protein secretome of these cells has been little investigated [13]. Using a combination of LC-MS/MS and bioinformatic analysis with confirmation by Western blotting we have identified several markers in the secretome indicative of healthy pluripotent stem cells or early (48h) pluripotency dissolution. Importantly these secretome markers were validated independently in hESC lines other than the iPSC and hESC lines Rebl.Pat and Man13 selected for the LC/MS interrogation. Moreover we have also shown that cells returned to E8 after 48h in E6 show recovery and exhibit secretome marker proteins correlating with the pluripotent state again.

The transmembrane FGF2 receptor FGFR1 was one of the most highly represented secreted markers for pluripotency dissolution. There are several possibilities to explain the increased incidence of FGFR1 in the conditioned E6 medium. Firstly, FGFR1 is known to be released into the extracellular space by cleavage in its membrane-adjacent extracellular domain by MMP2 [29]. As FGFR1 proteins are endocytosed by the cell upon substrate binding and degraded in lysosomes [30-32], it stands to reason that decreased FGF2 abundance may result in an increased surface abundance of FGFR1, and a corresponding increase in the amount of FGFR1 which is cleaved by surface metalloproteinases. Indeed, a majority of the FGFR1 peptides identified as significantly increased in E6 conditions in our proteomics study were N-terminal to the proposed MMP2 cleavage site. As it has previously been reported that a secreted form of the extracellular region of FGFR1 inhibits FGF2 activity [33] it could be speculated that the FGFR1 accumulation in FGF2 deficient media may further inhibit residual FGF2 signalling. Indeed, the biological functions of the E6-enriched protein biomarkers are consistent with a hypothesis that cells triggered towards differentiation release factors into the medium that bind and modulate growth factors, sequestering pluripotency promoting factors such as FGF2 and TGFβ1 reducing their local availability, and enhancing the signalling of other pro-differentiation factors. This will generate a feed forward amplification of the original pluripotency dissolution signal. Follistatin is a well-known stem cell differentiation factor, and TGFβ inhibitor, functioning through the binding and neutralisation of TGFβ superfamily members [16]. Similarly OLFML3, also enriched in E6, has been shown to bind to and stabilise BMP4, enhancing SMAD1/5/8 signalling in endothelial cells [34]. As BMP4 initiates differentiation of human embryonic stem cells to trophoblast cells in the absence of FGF [35-37], it could be concluded that after 48 hours of FGF2/TGFβ loss, these cells are secreting factors to promote, differentiation in nearby cells and commit them to this state [38].

SEMA3A, a neuronal signalling protein involved in axon guidance, is highly enriched in the E8 condition by both secretomics and RNAseq, whilst its transmembrane receptor, NRP1 is highly enriched (q<0.01) in the E6 transcriptome [39-41]. This is significant as NRP1 also binds to both FGF2 and TGFβ and modulates their signalling. In HUVEC cells, increased NRP1 expression was demonstrated to suppress Smad2/3 activation upon TGFβ-β stimulation, and expression of SMAD target genes in response to TGFβ was increased in NRP1 deficient HUVEC cells [42], a relationship which has been replicated in vivo in murine endothelial cells [43]. As NRP1 is a highly promiscuous receptor, ligands compete for NRP1 binding [44]. It has been demonstrated that ligand-competition exists between SEMA3A and VEGFA [44], and VEGFA and TGFβ [45]. Although experimental evidence would need to confirm this, this suggests that SEMA3A may compete with TGFβ for NRP1 binding in a manner which prevents TGFB sequestration and negative modulation by NRP1.

CHGA is a precursor protein for several neuroendocrine signalling proteins. Peptides from across the whole CHGA molecule were almost universally identified as being highly enriched in E8 conditioned medium over E6 conditioned medium. However the association with the pluripotent state is unclear. Generally speaking, CHGA drives formation and release of secretory granules [46, 47], and its decreased abundance in stem cells during pluripotency dissolution could reflect a reduction in overall secretion. This would be consistent with the greater number of secreted proteins identified in E8 conditioned medium compared with E6 (FIG. 2B).

NID1 is a secreted glycoprotein with two principle protein-binding domains (G2 and G3), separated by a flexible chain [48]. By domain-specific binding of different components of the basement membrane, NID1 stabilises the basement membrane by cross-linking its multiple components [49, 50]. NID1 is abundant in locations where there is a requirement for additional resilience against mechanical stress, and conversely, less abundant where more flexibility is required and tissue disposition is not fixed [51] for instance being actively degraded during basement membrane disassembly [52]. It has been demonstrated by proteomic analysis to be expressed at significant amounts by hESCs [13, 53]. In our current data we observe a dramatic reduction in the abundance of NID1 in E6 media compared with E8. As NID1 has a stabilising effect on the basement membrane, it is possible that it's abundance is decreased prior to the epithelial- mesenchymal transition inherent in the progression from a pluripotent stem cell to an early progenitor. This will involve the disassembly of a number of extracellular matrix complexes: in ovarian cancer cells Nidogen plays a key role in this EMT transition [54].

Although COCH is known to be secreted [55], relatively little is known about its function beyond its role in the extracellular matrix of the inner ear [56]. In murine ES cell COCH has been reported to be expressed in response to BMP4 signalling and this was suggested to support self-renewal [57]. In our study COCH is enhanced in E8 medium. The acknowledged molecular difference in the pluripotent ESC state between human and murine reflect an earlier naïve murine ESC phenotype and later primed human ESC phenotype [58] and are supported by the wealth of data on the differences in signalling required for murine and human stem cell maintenance [14, 15, 59, 60]. Thus BMP signalling has different effects in hESCs and murine ESCs, being active in stem cell maintenance in the latter [36, 60]. Since BMP signalling is strongly associated with differentiation to mesodermal and trophectodermal lineages in hESCs [35, 61-64], this would be in keeping with a correlation between targets such as COCH down stream of BMP and the dissolution of pluripotency, precipitating incipient lineage differentiation.

There is very little information on the function of NPTX2 outside of it's binding to α-amino-3-hydroxy-5-methyl-4-isoxazolepropionic acid (AMPA) receptor subunit Glutamate receptor 4 (GRIA4) in neuronal tissues [65]. It is certainly noteworthy that the transcript expression of GRIA4 is also much higher in E8 than E6 [data not shown].

### Discussion of Benefit of Paired-Ratio Method

A caveat of these analyses, which is encountered in all secretomic studies, is the lack of a suitable normalisation standard against which to compare proteins in order to robustly identify changes between conditions. Typically, ‘omics’ studies normalise to total protein or mRNA, or to a house-keeping gene or protein such as GAPDH or Actin. These controls allow for reliable, reproducible comparisons across conditions or experiments. When analysing the secreted complement of the cell proteome however, no such house-keeping protein exists, and the overall protein abundance between conditions is highly susceptible to variability due to the presence and concentration of medium proteins, the density of cells, differing cell viability, cytoplasmic leakage, and a host of other factors. In this study marker proteins were identified which could be multiplexed against each other to provide ratios which are much more discriminatory between the experimental conditions. It is believed that regardless of other changes in the cell secretome, the relative abundances of these proteins should remain relatively constant within an individual genetic background. As such, once an individual cell-line's baseline ratios are identified under control conditions, it is expected that these ratios would provide a rapid, quantitative warning of imminent pluripotency loss in cultured PSCs, above and beyond any current method in sensitivity, affordability and rapid timescale.

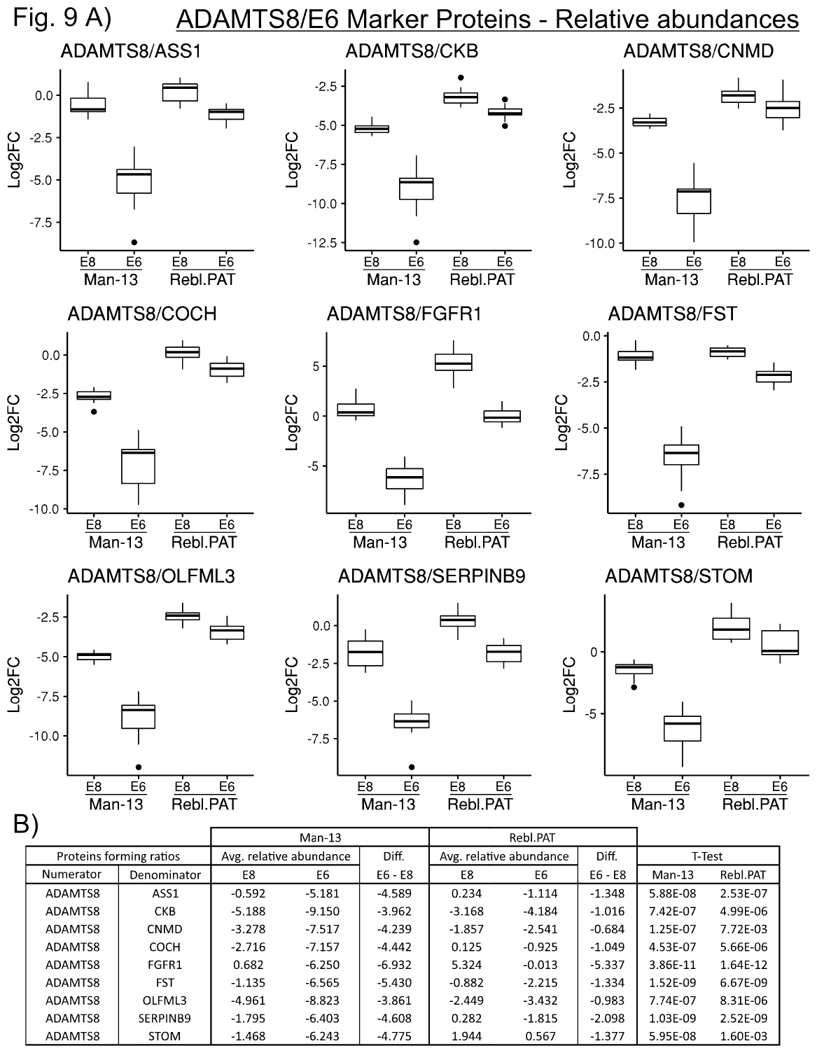

In summary it has been shown that there are a number of proteins in the secretome of PSCs that are rapidly decreased or increased during dissolution of pluripotency. By employing a matrix of four of each such species robust ratios of proteins could be generated which were indicative of stable pluripotency or its incipient loss. Additionally, further such ratios were identified by MS/MS (FIG. 9-32) and will make excellent targets for follow-up analyses. It is considered that such proteins will be invaluable as markers allowing intermittent or continuous culture monitoring during the scale up and manufacture of PSCs for cell therapy or for their use in pharmaceutical drug development and toxicity testing. However, given cost effective means of monitoring such markers they will be equally valuable in the research laboratory, allowing rapid evaluation of culture status without loss of cell product in either context.

### Methods

### Cell Culture

Cells were maintained in Essential 8 (E8) medium (Life Technologies) on Vitronectin-N (Life Technologies) and passaged using TrypLE dissociation reagent. Prior to sample collection for secretome analysis, cells were plated in E8 medium, supplemented for 24 hours with 10 μM Rock inhibitor, at a density of 20,000 (Rebl.PAT) or 25,000 (Man-13) cells per cm² in 75 cm² culture flasks (Corning). 24 hours after plating (experimental time 0), cells were either fed with fresh E8 medium, or transferred to Essential 6 (E6) medium (Life Technologies). Cells were fed at 48 and 72 hours (experimental time 24 h and 48 h respectively) after plating and at these time-points medium was collected, spun at 1,000×g at −9° C. for 5 minutes then aliquoted into lo-bind 2 ml centrifuge tubes (Eppendorf) and snap frozen in liquid nitrogen. At 72 hours after plating (experimental time 48 h), cells were dissociated with TrypLE, spun at 1,000×g in PBS for 5 minutes, the supernatant removed, and the pellet snap frozen in liquid nitrogen. A subset of the cells at this time-point were also collected and processed for RNA-Seq analysis. Five biological replicates were conducted for Rebl.PAT, each of which was composed of three technical replicates, and all samples were analysed by LC-MS/MS in a single run. In order to ensure quantitative reproducibility of the marker proteins identified across multiple analyses, four Man-13 biological replicates were collected, and these were analysed in two independent LC-MS/MS runs consisting of two biological replicates each. An important component of an assay for decreased PSC culture quality is the ability to detect a degradation of culture quality while the cells are recoverable. The experiments above were repeated but after 2 days in E6, cells were restored to E8 medium for 7 days to assess whether full pluripotency could be recovered. Medium was refreshed every 48 hrs with samples of conditioned medium being taken for analysis by Western Blot every 24 hours.

### Immunochemical Staining of Pluripotency Markers

Cells were seeded at a density of 20,000 (Rebl.PAT) or 25,000 (Man-13) cells per cm² in a 24 well plate and treated identically to samples prepared for proteomic and RNA-Seq analysis (FIG. 1A). Cell surface markers and transcription factors characteristic of pluripotent hESCs were detected using immunocytochemistry. The cells were fixed with 4% paraformaldehyde and incubated with antibodies against stage specific embryonic antigens SSEA-4, SSEA-1, TRA-1-60, TRA-1-81 (Abcam) and transcription factors SOX2, NANOG (Cell signalling Technologies), and OCT-4 (BD Biosciences) at 4° C. overnight. Secondary antibodies (Life Technologies), specific for the species and isotype of the primary antibody, conjugated to FITC or TRITC were used for detection using a BX51 microscope (Olympus, Hertfordshire, UK) equipped with a Q-Imaging camera (Micro Imaging Applications Group, Inc, Buckinghamshire, UK). Image processing was done with the aid of Q-Capture Pro software package (Micro Imaging Applications Group, Inc).

### Flow Cytometry

Cells were collected at the time of plating, and at the 48-hour time-point for each experiment, and at the 24- hour time-point for a subset of experiments. All flow cytometry analyses were performed on a BD LSRFortessa™ cell analyser (Becton-Dickinson, San Jose, CA). Cells were dissociated using TrypLE Express (Invitrogen), fixed in the dark for 7 minutes at room temperature in 4% paraformaldehyde, blocked for two hours in 5% FBS and were permeabilised for intracellular staining in ice-cold 70% methanol. All antibodies used were directly conjugated with fluorochrome. Species and fluorochrome-matched isotype controls were used for each antibody to control for non-specific binding.

Cells were incubated with primary antibodies for 30 minutes at room temperature, at the following dilutions; Phycoerythrin-conjugated mouse α-TRA 1-81 (Ebioscience 12-8883-80), 1:300; Phycoerythrin-conjugated mouse isotype control (Santa-Cruz, SC2870) 1:75; Alexa 488-conjugated Mouse α-Nanog (BD Bioscience, BD560791), 1:10; Alexa 488-conjugated mouse isotype control (BD Bioscience, BD557702) 1:100; Alexa 647-conjugated mouse α-Sox2 (BD bioscience, BD56139) 1:80; Alexa 647-conjugated mouse isotype control (BD Bioscience, BD557714) 1:80. Flow cytometry data was collected and preliminarily processed in FACSDIVA™ software followed by analysis and figure-preparation in FlowJo (both Becton-Dickinson, San Jose, Calif.).

### Proteomics Sample Preparation

Media samples were digested using a modified Filter-assisted Sample Preparation (FASP) method with the following modification: The samples were concentrated to approximately 50 μL with Microcon-10 kDa centrifugal filter units (Merck Millipore) at a speed of 14,000×g. This was then buffer-exchanged by washing and centrifuging three times with the addition of 100 μL of 25 mM ammonium bicarbonate before the proteins were reconstituted in 50 μL of 25 mM ammonium bicarbonate. The protein concentration was determined using Millipore Direct Detect® spectrometer and 50 μg (Man-13) or 25 μg (Rebl.PAT) of protein was added to a fresh 10 kDa filter tube with reduction, alkylation and digestion occurring using the filter tubes. After digestion peptides were collected by centrifugation and the samples were desalted with OLIGO™ R3 reversed-phase media on a microplate system and reconstituted in 5% acetonitrile and 0.1% formic acid.

### LC-MS/MS

Digested samples were analysed by LC-MS/MS using an UltiMate 3000 Rapid Separation LC (RSLC, Dionex Corporation, Sunnyvale, Calif.) coupled to an Orbitrap Elite (Thermo Fisher Scientific, Waltham, Mass.) mass spectrometer. Peptide mixtures were separated using a gradient from 5% to 33% acetonitrile in 0.1% formic acid over 44 min at 300 nL min⁻¹, using a 250 mm×75 μm i.d. 1.7 mM BEH C18, analytical column (Waters). Peptides were selected for fragmentation automatically in a data dependant manner.

### Identification and Quantification of Peptides

The acquired MS data was analysed using Progenesis LC-MS (v4.1, Nonlinear Dynamics). The retention times in each sample were aligned using one LC-MS run as a reference, then the “Automatic Alignment” algorithm was used to create maximal overlay of the two-dimensional feature maps, where necessary a minimal amount of manual adjustment was employed to increase alignment score to above 80%. Features with charges +5 were masked and excluded from further analyses, as were features with less than 3 isotope peaks. The resulting peak lists were searched against the SwissProt (release 2016-04) and Trembl (release 2016-04) databases using Mascot v2.5.1, (Matrix Science). Search parameters included a precursor tolerance of 5 ppm and a fragment tolerance of 0.6 Da. Enzyme specificity was set to trypsin and one missed cleavage was allowed. Carbamidomethyl modification of cysteine was set as a fixed modification while methionine oxidation was set to variable. The Mascot results were imported into Progenesis LC-MS for annotation of peptide peaks.

### Proteomics Data Processing

Swissprot and Trembl IDs were used to retrieve gene names, Uniprot IDs, peptide sequences and Entrez IDs from the Uniprot database. For Uniprot entries which lacked an Entrez ID these were identified either by searching gene names against the online annotation database DAVID, or by manual curation using gene names and descriptions. Duplicate Entrez IDs were consolidated into a single entry by the retention of the entry with the highest quantification confidence (presence of 2 or more unique peptides, or highest confidence score).

TABLE 1
Corresponding gene name, protein name and Uniprot ID

| GN.1 | Uniprot/Swissprot all | Man.13_R1_Accession | Man.13_R2_Accession | R.PAT_Accession |
|---|---|---|---|---|
| ADAMTS8 | 1::ATS8_HUMAN; | 1::ATS8_HUMAN | 1::ATS8_HUMAN | 2::Q5FWF1_ HUMAN |
| | 2::Q5FWF1_HUMAN | | | |
| APP | 1::A4_HUMAN; | 1::A4_HUMAN; | 1::A4_HUMAN; | 1::A4_HUMAN; |
| | 2::A0A0A0MRG2_HUMAN; | 2::A0A0A0MRG2_HUMAN; | 2::A0A0A0MRG2_HUMAN; | 2::A0A0A0MRG2_HUMAN; |
| | 2::B4DGD0_HUMAN; | 2::B4DGD0_HUMAN; | 2::B4DGD0_HUMAN; | 2::B4DGD0_HUMAN; |
| | 2::B4DJT9_HUMAN; | 2::B4DJT9_HUMAN; | 2::B4DJT9_HUMAN; | 2::B4DM00_HUMAN; |
| | 2::B4DM00_HUMAN; | 2::B4DM00_HUMAN; | 2::B4DM00_HUMAN; | 2::B4DMD5_HUMAN; |
| | 2::B4DMD5_HUMAN; | 2::B4DMD5_HUMAN; | 2::B4DMD5_HUMAN; | 2::B4DQM1_HUMAN; |
| | 2::B4DQM1_HUMAN; | 2::B4DQM1_HUMAN; | 2::B4DQM1_HUMAN; | 2::B7Z313_HUMAN; |
| | 2::B7Z313_HUMAN; | 2::B7Z313_HUMAN; | 2::B7Z313_HUMAN; | 2::E9PG40_HUMAN; |
| | 2::H7C0V9_HUMAN; | 2::H7C0V9_HUMAN; | 2::E9PG40_HUMAN; | 2::H7C0V9_HUMAN; |
| | 2::H7C2L2_HUMAN; | 2::H7C2L2_HUMAN; | 2::H7C0V9_HUMAN; | 2::H7C2L2_HUMAN |
| | 2::L7XE61_HUMAN; | 2::L7XE61_HUMAN | 2::H7C2L2_HUMAN; | |
| | 2::E9PG40_HUMAN | | 2::L7XE61_HUMAN | |
| B3GNT7 | 1::B3GN7_HUMAN | 1::B3GN7_HUMAN | 1::B3GN7_HUMAN | 1::B3GN7_HUMAN |
| CHGA | 1::CMGA_HUMAN; | 1::CMGA_HUMAN; | 1::CMGA HUMAN; | 1::CMGA_HUMAN; |
| | 2::G5E968_HUMAN | 2::G5E968_HUMAN | 2::G5E968_HUMAN | 2::G5E968_HUMAN |
| EFEMP1 | 2::A0A0S2Z4F1_HUMAN; | 2::A0A0S2Z4F1_HUMAN; | 2::A0A0S2Z4F1_HUMAN; | 2::A0A0S2Z4F1_HUMAN; |
| | 2::A0A0S2Z3V1_HUMAN; | 2::A0A0S2Z3V1_HUMAN; | 2::A0A0S2Z3V1_HUMAN; | 2::A0A0S2Z3V1_HUMAN; |
| | 2::A0A0U1RQV3_HUMAN; | 2::B2R6M6_HUMAN; | 2::A0A0U1RQV3_HUMAN; | 2::B3KS53_HUMAN; |
| | 2::B2R6M6_HUMAN; | 2::B3KS53_HUMAN; | 2::B2R6M6_HUMAN; | 2::C9J4J8_HUMAN; |
| | 2::B3KS53_HUMAN; | 2::C9J4J8_HUMAN; | 2::C9J4J8_HUMAN; | 2::C9JUM4_HUMAN; |
| | 2::C9J4J8_HUMAN; | 2::C9JUM4_HUMAN; | 2::C9JUM4_HUMAN; | 2::Q53TA7_HUMAN; |
| | 2::C9JUM4_HUMAN; | 2::Q53TA7_HUMAN | 2::Q53TA7_HUMAN; | 2::Q580Q6_HUMAN; |
| | 2::Q53TA7_HUMAN; | | 2 ::Q59G97_HUMAN | 2::Q59G97_HUMAN |
| | 2::Q58006HUMAN; | | | |
| | 2::Q59G97_HUMAN | | | |
| EPHA1 | 2::A8K2T6_HUMAN; | 2::A8K2T6_HUMAN; | 2::A8K2T6_HUMAN; | 1 ::EPHA1_HUMAN |
| | 1::EPHA1_HUMAN | 1::EPHA1_HUMAN | 1::EPHA1_HUMAN | |
| EXTL2 | 1::EXTL2_HUMAN; | 1::EXTL2_HUMAN; | 1::EXTL2_HUMAN; | 1::EXTL2_HUMAN; |
| | 2::B4DNZ2_HUMAN; | 2::B4DNZ2_HUMAN; | 2::B4DNZ2_HUMAN; | 2::B4DNZ2_HUMAN; |
| | 2::C9IYF5_HUMAN; | 2::C9IYF5_HUMAN; | 2::C9IYF5_HUMAN; | C9IY2::F5_HUMAN; |
| | 2::C9JEG3_HUMAN; | 2::C9JEG3_HUMAN; | 2::O00245_HUMAN | 2::C9JEG3_HUMAN; |
| | 2::O00245_HUMAN | 2::O00245_HUMAN | | 2::O00245_HUMAN |
| FBLN2 | 1::FBLN2_HUMAN; | 2::B7Z9B8_HUMAN; | 1::FBLN2_HUMAN; | 1::FBLN2_HUMAN; |
| | 2::B7Z9B8_HUMAN; | 2::B7Z6T9_HUMAN; | 2::B7Z6T9_HUMAN; | 2::B7Z6T9_HUMAN |
| | 2::B7Z6T9_HUMAN; | 2::C9JQS6_HUMAN | 2::C9JQS6_HUMAN | |
| | 2::C9JQS6_HUMAN | | | |
| FGF2 | 2::A0A087WUF6_HUMAN; | 2::A0A087WUF6_HUMAN | 2::D9ZGF5_HUMAN | 2::A0A087WUF6_HUMAN |
| | 2::D9ZGF5_HUMAN | | | |
| FUCA2 | 1 ::FUCO2_HUMAN; | 1 ::FUCO2_HUMAN; | 1::FUCO2_HUMAN | 1::FUCO2_HUMAN |
| | 2::Q7Z6V2_HUMAN | 2::Q7Z6V2_HUMAN | | |
| GALNT1 | 2::A0A024R048_HUMAN; | 2::A0A024R048_HUMAN; | 2::A0A024R048_HUMAN; | 2::A0A024R048_HUMAN; |
| | 2::A8KAJ7_HUMAN; | 2::K7EJV8_HUMAN; | 2::Q68VJ7_HUMAN | 2::A8KAJ7_HUMAN; |
| | 2::K7EJV8_HUMAN; | 2::Q68VJ7_HUMAN | | 2::K7EJV8_HUMAN; |
| | 2::Q68VJ7_HUMAN | | | 2::Q68VJ7_HUMAN |
| GGH | 1::GGH_HUMAN; | 1::GGH_HUMAN; | 1::GGH_HUMAN; | 1::GGH_HUMAN; |
| | 2::B4DVI2_HUMAN | 2::B4DVI2_HUMAN | 2::B4DVI2_HUMAN | 2::B4DVI2_HUMAN |
| HEXB | 2::A0A024RAJ6_HUMAN; | 2::A0A024RAJ6_HUMAN; | 2::A0A024RAJ6_HUMAN; | 2::H0YA83_HUMAN |
| | 2::D6REQ8_HUMAN; | 2::D6REQ8_HUMAN; | 2::D6REQ8_HUMAN; | |
| | 2::H0Y9B6_HUMAN | 2::H0Y9B6_HUMAN | 2::H0Y9B6_HUMAN | |
| IGFBP4 | 2::A0A024R1U8_HUMAN | 2::A0A024R1U8_HUMAN | 2::A0A024R1U8_HUMAN | 2::A0A024R1U8_HUMAN |
| NID1 | 1::NID1_HUMAN; | 1::NID1_HUMAN; | 1::NID1_HUMAN; | 1::NID1 HUMAN |
| | 2::B4DM05_HUMAN | 2::B4DM05_HUMAN | 2::B4DM05_HUMAN | |
| NPTX2 | 1::NPTX2_HUMAN | 1::NPTX2_HUMAN | 1::NPTX2_HUMAN | 1::NPTX2_HUMAN |
| NTS | 1::NEUT_HUMAN | 1::NEUT_HUMAN | 1::NEUT_HUMAN | 1::NEUT_HUMAN |
| PCDHB5 | 1::PCDB5_HUMAN; | 1::PCDB5_HUMAN; | 1::PCDB5_HUMAN; | 1::PCDB5_HUMAN |
| | 2::A0A096LP94_HUMAN | 2::A0A096LP94_HUMAN | 2::A0A096LP94_HUMAN | |
| SEMA3A | 1::SEM3A_HUMAN; | 1::SEM3A_HUMAN; | 1::SEM3A_HUMAN; | 1::SEM3A_HUMAN; |
| | 2::C9J9C4_HUMAN; | 2::C9J904_HUMAN; | 2::C9J9C4_HUMAN; | 2::C9J9C4_HUMAN; |
| | 2::Q75MQ2_HUMAN; | 2::Q75MQ2_HUMAN; | 2::Q75MQ2_HUMAN; | 2::Q75MQ2_HUMAN; |
| | 2::Q86UJ2_HUMAN | 2::Q86UJ2_HUMAN | 2::Q86UJ2_HUMAN | 2::Q86UJ2_HUMAN |
| SEMA3F | 2::Q59G50_HUMAN; | 2::Q59G50_HUMAN; | 2::Q59G50_HUMAN; | 1::SEM3F_HUMAN; |
| | 1::SEM3F_HUMAN; | 1::SEM3F_HUMAN; | 1::SEM3F_HUMAN; | 2::H704A2_HUMAN |
| | 2::C9J4H5_HUMAN; | 2::C9J4H5_HUMAN | 2::C9J1V2_HUMAN | |
| | 2::C9J1V2_HUMAN; | | | |
| | 2::H7C4A2_HUMAN | | | |
| SFRP2 | 1::SFRP2_HUMAN; | 1::SFRP2_HUMAN | 1::SFRP2_HUMAN | 1::SFRP2_HUMAN; |
| | 2::B3KSM5_HUMAN | | | 2::B3KSM5_HUMAN |
| TIMP4 | 1::TIMP4_HUMAN | 1::TIMP4_HUMAN | 1::TIMP4_HUMAN | 1::TIMP4_HUMAN |
| TNFRSF8 | 1::TNR8_HUMAN | 1::TNR8_HUMAN | 1::TNR8_HUMAN | 1::TNR8_HUMAN |
| WFDC2 | 1::WFDC2_HUMAN; | 1::WFDC2_HUMAN | 1::WFDC2_HUMAN | 1::WFDC2_HUMAN; |
| | 2::A8K2M3_HUMAN | | | 2::A8K2M3_HUMAN |
| GN.2 | Uniprot/Swissprot all | Man.13_R1_Accession | Man.13_R2_Accession | R.PAT_Accession |
| ASS1 | 1::ASSY_HUMAN; | 1::ASSY_HUMAN; | 1::ASSY_HUMAN; | 1::ASSY_HUMAN |
| | 2::A0A0S2Z3B6_HUMAN; | 2::A0A0S2Z3B6_HUMAN; | 2::A0A0S2Z3B6_HUMAN; | |
| | 2::B4E395_HUMAN | 2::B4E395_HUMAN | 2::B4E395_HUMAN | |
| CKB | 1::KCRB_HUMAN; | 1::KCRB_HUMAN; | 1::KCRB_HUMAN; | 1::KCRB_HUMAN; |
| | 2::A0A052Z471_HUMAN; | 2::B4DP56_HUMAN; | 2::A0A052Z471_HUMAN; | 2::A0A052Z471_HUMAN; |
| | 2::B4DP56_HUMAN; | 2::G3V461_HUMAN; | 2::B4DP56_HUMAN; | 2::B4DP56_HUMAN; |
| | 2::G3V2I1_HUMAN; | 2::G3V4N7_HUMAN; | 2::G3V461_HUMAN; | 2::G3V2I1_HUMAN; |
| | 2::G3V461_HUMAN; | 2::H0YJG0_HUMAN; | 2::G3V4N7_HUMAN; | 2::G3V461_HUMAN; |
| | 2::G3V4N7_HUMAN; | 2::H0YJK0_HUMAN | 2::H0YJG0_HUMAN | 2::G3V4N7_HUMAN; |
| | 2::H0YJG0_HUMAN; | | | 2::H0YJG0_HUMAN |
| | 2::H0YJK0_HUMAN | | | |
| CNMD | 1::LECT1_HUMAN; | 1::LECT1_HUMAN; | 1::LECT1_HUMAN; | 1::LECT1_HUMAN; |
| | 2::E9PKI9_HUMAN; | 2::E9PKI9_HUMAN; | 2::E9PKI9_HUMAN; | 2::E9PKI9_HUMAN; |
| | 2::Q6UJZ0_HUMAN | 2::Q6UJZ0_HUMAN | 2::Q6UJZ0_HUMAN | 2::Q6UJZ0_HUMAN |
| COCH | 1::COCH_HUMAN | 1::COCH_HUMAN; | 1::COCH_HUMAN; | 1::COCH_HUMAN; |
| | | 2::B3KVT3_HUMAN; | 2::B3KVT3_HUMAN; | 2::B3KVT3_HUMAN; |
| | | 2::G3V4C4_HUMAN; | 2::G3V4C4_HUMAN; | 2::G3V4C4_HUMAN; |
| | | 2::G3V5G6_HUMAN | 2::G3V5G6_HUMAN; | 2::G3V5V4_HUMAN; |
| | | | 2::G3V5V4_HUMAN; | 2::G3V5X3_HUMAN; |
| | | | 2::G3V5X3_HUMAN; | 2::H0YJW4_HUMAN |
| | | | 2::H0YJW4_HUMAN | |
| FGFR1 | 1::FGFR1_HUMAN; | 2::A0A0S2Z3P4_HUMAN | 2::A0A0S2Z3T4_HUMAN; | 2::A0A052Z3Q6_HUMAN; |
| | 2::A0A0S2Z3P4_HUMAN; | | 1::FGFR1_HUMAN; | 2::A0A0S2Z3P4_HUMAN; |
| | 2::A0A0S2Z3Q6_HUMAN; | | 2::B5A958_HUMAN; | 2::B5A958_HUMAN; |
| | 2::A0A0S2Z3T4_HUMAN; | | 2::E9PKF2_HUMAN; | 2::09J205_HUMAN; |
| | 2::B5A958_HUMAN; | | 2::E9PKV7_HUMAN; | 2::E9PNM3_HUMAN |
| | 2::C9J205_HUMAN; | | 2::E9PN14_HUMAN; | |
| | 2::E9PKF2_HUMAN; | | 2::E9PQ40_HUMAN | |
| | 2::E9PKV7_HUMAN; | | | |
| | 2::E9PN14_HUMAN; | | | |
| | 2::E9PNM3_HUMAN; | | | |
| | 2::E9P040_HUMAN | | | |
| FST | 2::A0A024QZU6_HUMAN; | 2::Q6FHE1_HUMAN | 2::A0A024QZU6_HUMAN; | 2::A0A024QZU6_HUMAN; |
| | 2::H0YA75_HUMAN; | | 2::H0YA75_HUMAN; | 2::H0YA75_HUMAN; |
| | 2::H0YAF9_HUMAN; | | 2::H0YAF9_HUMAN; | 2::H0YAF9_HUMAN; |
| | 2::Q6FHE1_HUMAN | | 2::Q6FHE1_HUMAN | 2::Q6FHE1_HUMAN |
| OLFML3 | 2::M1LAK4_HUMAN; | 2::M1LAK4_HUMAN; | 2::M1LAK4_HUMAN; | 2::M1LAK4_HUMAN |
| | 2::B4DNG0_HUMAN | 2::B4DNG0_HUMAN | 2::B4DNG0_HUMAN | |
| SERPINB9 | 2::A0A024QZT4_HUMAN; | 2::A0A024QZT4_HUMAN; | 2::A0A024QZT4_HUMAN; | 2::A0A024QZT4_HUMAN; |
| | 1::SPB8_HUMAN; | 1::SPB8_HUMAN; | 2::Q6N0A8_HUMAN | 2::Q6N0A8_HUMAN |
| | 2::Q6N0A8_HUMAN | 2::Q6N0A8_HUMAN | | |
| STOM | 2::A0A024R882_HUMAN; | 2::A0A024R882_HUMAN | 2::A0A024R882_HUMAN; | 2::A0A024R882_HUMAN; |
| | 2::B4E310_HUMAN; | | 2::B4E310_HUMAN | 2::B4E310_HUMAN; |
| | 2::F8VSL7_HUMAN | | | 2::F8VSL7_HUMAN |

### Secretion Pathways

Peptide sequences for all proteins were uploaded to the SignalP 4.1 server [66] to identify the proportion of proteins bearing a classical secretion signal-peptide sequence. Non-classical secretion was identified by uploading peptide sequences to the SecretomeP 2.0 server [67]; and the recommended cut-off score of 0.6 was used to quantify the proportion of proteins which were likely to be secreted through non-classical pathways. Extra-cellular-associated GO-terms were identified by uploading Entrez IDs for all proteins to DAVID Bioinformatics Resource 6.8 [68], and protein lists were retrieved for proteins with the GO terms; cell surface, extracellular region, extracellular space, extracellular matrix, and extracellular vesicle.

### Statistical Analyses

We Log2 transformed all abundance data to improve the normality of it's distribution, thus improving the power of the statistical tests and reducing data skew. Proteomics p-values (Welch's T-Test) and q-values (Benjamini-Hochberg method multiple comparison adjustment) were both calculated in R using functions t.test( ) and p.adjust(method=“fdr”).

### Marker Identification Using Protein-Protein Ratio Abundance

To identify novel protein-pair relationships which are indicative of change to cell pluripotency, all normalised protein abundance data for proteins with Progenesis confidence score over 30 were inputted into a custom R script which calculated the protein-pair combinations for every sample of every protein. The p-values and FDRs for every ratio between E8 and E6 were calculated. Ratio pairs with a highly significant change between E8 and E6 conditions were expressed as a network using Cytoscape [69]. The 30 proteins which formed the most altered protein-pair combinations with other proteins were isolated and their interactions, biological functions and relative abundances were assessed individually.

### Western blotting

Media from a variety of cell lines, including Man-1, Man-7 and H9 cells in addition to MAN-13 and Rebl.PAT, was collected as described in FIG. 1A, and 3 ml of media was concentrated to 50-100 μl in Microcon—10 kDa centrifugal filter units (Merck Millipore). Samples were run on 10% Bis-Tris gels (Thermo #NW00100BOX) run on a Mini Gel Tank (Thermo #A25977) using MES SDS Running Buffer (Thermo #B0002) alongside broad range markers (11-245KDa, NEB #P7712S). Each lane contained 20 μg of protein, heated in Pierce lane marker reducing buffer (Thermo #39000) at 95° C. for 10 minutes. Gels were transferred using the IBlot2 Gel Transfer device (Thermo #IB21001), using iBlot 2 Transfer stacks (nitrocellulose membrane, Thermo #IB23001).Cells were analysed by immunoblotting using the following antibodies: 1/200 Mouse α-Chromogranin A (Novus Biologicalis, NBP2-44774); 1/100 Rabbit α-COCH Abcam, ab170266); 1/500 mouse α-FGFR1 (R&D systems, MAB658); 1/1000 rabbit α-Follistatin (Abcam, ab157471); 1/1000 rabbit α-Entactin/NID1 (Abcam, ab133686); rabbit α-Neuronal Pentaxtrin 2 (Abcam, ab191563); 1/1000 rabbit α-Neurotensin (Abcam, ab172114); 1/300 rabbit α-Olfactomedin-like 3 (Abcam, ab111712); 1/500 mouse α-Semaphorin 3A (R&D, MAB1250); 1/500 mouse α-Secreted Frizzled Related Protein 2 (R&D, MAB6838). Secondary antibody staining was performed either using 1/20,000 IRDye 800CW Goat α-mouse, or 1/20,000 IRDye 680RD Donkey α-Rabbit (P/N 925-32210 and 925-68071 respectively, both from LI-COR). These fluorescent secondary antibodies were imaged using the Odyssey CLx imaging system (LI-COR) typically in pairs of rabbit and mouse antibodies together. Image brightness/contrast adjustment and densitometry quantification was all performed in ImageJ (NIH).

### Assessment in TeSR1 Media

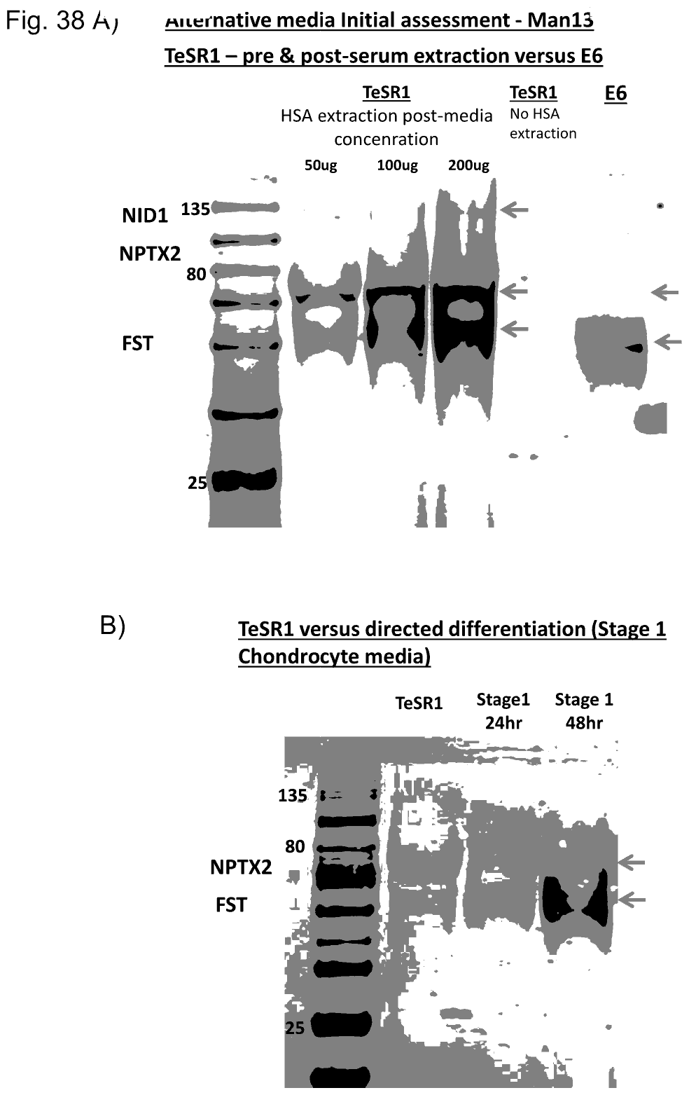

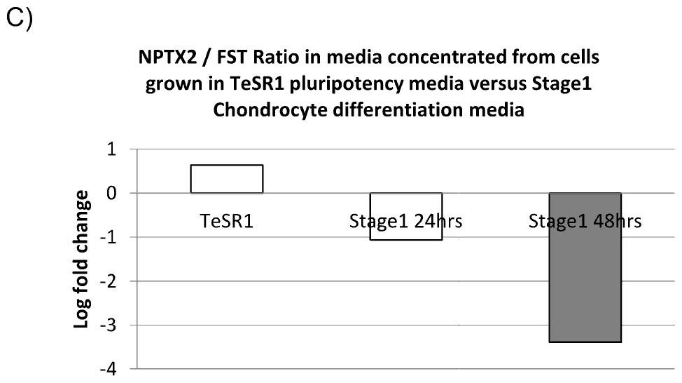

Man 13 Cell cultures were assessed in TeSR1 medium as shown in FIG. 38. This medium has high HSA which interferes with western blotting so it had to be removed before running the gel.

FIG. 38A) shows 3 different concentrations of this alternative, commonly used, medium TeSR1 (after HSA removal) at concentrations of 50 μug, 100 μg and 200 μg. NID1 and NPX2 can be seen in the TeSR medium although NID 1 was only detectable after running 200 μug after the HSA extraction (this may be partly removed with the HSA). E6 medium is detectable for only 20 μg protein loaded. There is a strong Follistatin band (this is increased on differentiation): which is also strongly detected using only 20 μg protein in the differentiation medium E6 and is stronger than with 50 μg TESR1.

FIG. 38B) shows TESR1 grown cells then 2 lanes after 24 and 48 h differentiation. The increased Follistin after 24 then 48 h. 38A) shows differentiation medium (a chondrogenic medium) with loss of NPTX2. The quantitation is in FIG. 38C and this shows NPTX2/FST Ratio in medum concentrated from cells grown in TeSR1 pluripotency media versus Stagel Chondrocyte differentiation media.

### REFERENCES

1. Trans-Proteomic Pipeline, a standardized data processing pipeline for large-scale reproducible proteomics informatics.

2. Schneider, C. A., W. S. Rasband, and K. W. Eliceiri, NIH Image to ImageJ: 25 years of image analysis. Nature Methods, 2012. 9 (7): p. 671-675.

3. Muller, F. J., et al., A bioinformatic assay for pluripotency in human cells. Nat Methods, 2011. 8 (4): p. 315-7.

4. Bock, C., et al., Reference Maps of human ES and iPS cell variation enable high-throughput characterization of pluripotent cell lines. Cell, 2011. 144 (3): p. 439-52.

5. Avior, Y., J. C. Biancotti, and N. Benvenisty, TeratoScore: Assessing the Differentiation Potential of Human Pluripotent Stem Cells by Quantitative Expression Analysis of Teratomas. Stem Cell Reports, 2015. 4 (6): p. 967-74.

6. Kerz, M., et al., A Novel Automated High-Content Analysis Workflow Capturing Cell Population Dynamics from Induced Pluripotent Stem Cell Live Imaging Data. J Biomol Screen, 2016. 21 (9): p. 887-96.

7. Perestrelo, T., et al., Pluri-IQ: Quantification of Embryonic Stem Cell Pluripotency through an Image-Based Analysis Software. Stem Cell Reports, 2017. 9 (2): p. 697-709.

8. Smith, D., K. Glen, and R. Thomas, Automated image analysis with the potential for process quality control applications in stem cell maintenance and differentiation. Biotechnol Prog, 2016. 32 (1): p. 215-23.

9. Munoz, J., et al., The quantitative proteomes of human-induced pluripotent stem cells and embryonic stem cells. Mol Syst Biol, 2011. 7: p. 550.

10. Phanstiel, D. H., et al., Proteomic and phosphoproteomic comparison of human ES and iPS cells. Nat Methods, 2011. 8(10): p. 821-7.

11. Yamana, R., et al., Rapid and deep profiling of human induced pluripotent stem cell proteome by one-shot NanoLC-MS/MS analysis with meter-scale monolithic silica columns. J Proteome Res, 2013. 12 (1): p. 214-21.

12. Pripuzova, N. S., et al., Development of a protein marker panel for characterization of human induced pluripotent stem cells (hiPSCs) using global quantitative proteome analysis. Stem Cell Res, 2015. 14 (3): p. 323-38.

13. Soteriou, D., et al., Comparative proteomic analysis of supportive and unsupportive extracellular matrix substrates for human embryonic stem cell maintenance. J Biol Chem, 2013. 288 (26): p. 18716-31.

14. Xu, R. H., et al., Basic FGF and suppression of BMP signaling sustain undifferentiated proliferation of human ES cells. Nat Methods, 2005. 2 (3): p. 185-90.

15. Beattie, G. M., et al., Activin A maintains pluripotency of human embryonic stem cells in the absence of feeder layers. Stem Cells, 2005. 23 (4): p. 489-95.

16. Vallier, L., M. Alexander, and R. A. Pedersen, Activin/Nodal and FGF pathways cooperate to maintain pluripotency of human embryonic stem cells. J Cell Sci, 2005. 118 (Pt 19): p. 4495-509.

17. Gonzales, K. A., et al., Deterministic Restriction on Pluripotent State Dissolution by Cell-Cycle Pathways. Cell, 2015. 162 (3): p. 564-79.

18. Ye, J., et al., High quality clinical grade human embryonic stem cell lines derived from fresh discarded embryos. Stem Cell Res Ther, 2017. 8 (1): p. 128.

19. Hammad, M., et al., Identification of polymer surface adsorbed proteins implicated in pluripotent human embryonic stem cell expansion. Biomater Sci, 2016. 4 (9): p. 1381-91.

20. Chen, G., et al., Chemically defined conditions for human iPSC derivation and culture. Nat Methods, 2011. 8 (5): p. 424-9.

21. Lippmann, E. S., M. C. Estevez-Silva, and R. S. Ashton, Defined human pluripotent stem cell culture enables highly efficient neuroepithelium derivation without small molecule inhibitors. Stem Cells, 2014. 32 (4): p. 1032-42.

22. Greber, B., et al., Conserved and divergent roles of FGF signaling in mouse epiblast stem cells and human embryonic stem cells. Cell Stem Cell, 2010. 6 (3): p. 215-26.

23. Kopp, J. L., et al., Small increases in the level of Sox2 trigger the differentiation of mouse embryonic stem cells. Stem Cells, 2008. 26 (4): p. 903-11.

24. Boer, B., et al., Elevating the levels of Sox2 in embryonal carcinoma cells and embryonic stem cells inhibits the expression of Sox2:Oct-3/4 target genes. Nucleic Acids Res, 2007. 35 (6): p. 1773-86.

25. Zhao, S., et al., SoxB transcription factors specify neuroectodermal lineage choice in ES cells. Mol Cell Neurosci, 2004. 27 (3): p. 332-42.

26. Thomson, M., et al., Pluripotency factors in embryonic stem cells regulate differentiation into germ layers. Cell, 2011. 145 (6): p. 875-89.

27. Wang, Z., et al., Distinct lineage specification roles for NANOG, OCT4, and SOX2 in human embryonic stem cells. Cell Stem Cell, 2012. 10 (4): p. 440-54.

28. Schultz, S. Determining the Predictive Mechanism of Toxicity Using a Single-Well Multiplexed Assay. 2009 [cited 2019; Available from: http://www.promega.co.uk/resources/pubhub/determining-the-predictive-mechanism-of-toxicity-with-apotox-glo/.

29. Levi, E., et al., Matrix metalloproteinase 2 releases active soluble ectodomain of fibroblast growth factor receptor 1. Proc Natl Acad Sci USA, 1996. 93 (14): p. 7069-74.

30. Haugsten, E. M., et al., Different intracellular trafficking of FGFI endocytosed by the four homologous FGF receptors. J Cell Sci, 2005. 118 (Pt 17): p. 3869-81.

31. Auciello, G., et al., Regulation of fibroblast growth factor receptor signalling and trafficking by Src and Eps8. J Cell Sci, 2013. 126 (Pt 2): p. 613-24.

32. Sorokin, A., et al., Internalization of fibroblast growth factor receptor is inhibited by a point mutation at tyrosine 766. J Biol Chem, 1994. 269 (25): p. 17056-61.

33. Guillonneau, X., et al., Fibroblast growth factor (FGF) soluble receptor 1 acts as a natural inhibitor of FGF2 neurotrophic activity during retinal degeneration. Mol Biol Cell, 1998. 9 (10): p. 2785-802.

34. Miljkovic-Licina, M., et al., Targeting olfactomedin-like 3 inhibits tumor growth by impairing angiogenesis and pericyte coverage. Mol Cancer Ther, 2012. 11 (12): p. 2588-99.

35. Pera, M. F., et al., Regulation of human embryonic stem cell differentiation by BMP-2 and its antagonist noggin. J Cell Sci, 2004. 117 (Pt 7): p. 1269-80.

36. Onishi, K., et al., Local BMP-SMAD1 signaling increases LIF receptor-dependent STAT3 responsiveness and primed-to-naive mouse pluripotent stem cell conversion frequency. Stem Cell Reports, 2014. 3 (1): p. 156-68.

37. Amita, M., et al., Complete and unidirectional conversion of human embryonic stem cells to trophoblast by BMP4. Proc Natl Acad Sci USA, 2013. 110 (13): p. E1212-21.

38. Xu, R. H., et al., BMP4 initiates human embryonic stem cell differentiation to trophoblast. Nat Biotechnol, 2002. 20 (12): p. 1261-4.

39. Janssen, B. J., et al., Neuropilins lock secreted semaphorins onto plexins in a ternary signaling complex. Nat

Struct Mol Biol, 2012. 19 (12): p. 1293-9.

40. Lepelletier, Y., et al., Immunosuppressive role of semaphorin-3A on T cell proliferation is mediated by inhibition of actin cytoskeleton reorganization. Eur J Immunol, 2006. 36 (7): p. 1782-93.

41. Catalano, A., The neuroimmune semaphorin-3A reduces inflammation and progression of experimental autoimmune arthritis. J Immunol, 2010. 185 (10): p. 6373-83.

42. Aspalter, I. M., et al., Alk1 and Alk5 inhibition by Nrp1 controls vascular sprouting downstream of Notch. Nat Commun, 2015. 6: p. 7264.

43. Hirota, S., et al., Neuropilin 1 balances beta8 integrin-activated TGFbeta signaling to control sprouting angiogenesis in the brain. Development, 2015. 142 (24): p. 4363-73.

44. Bachelder, R. E., et al., Competing autocrine pathways involving alternative neuropilin-1 ligands regulate chemotaxis of carcinoma cells. Cancer Res, 2003. 63 (17): p. 5230-3.

45. Glinka, Y. and G. J. Prud'homme, Neuropilin-1 is a receptor for transforming growth factor beta-1, activates its latent form, and promotes regulatory T cell activity. J Leukoc Biol, 2008. 84 (1): p. 302-10.

46. Stettler, H., et al., Determinants for chromogranin A sorting into the regulated secretory pathway are also sufficient to generate granule-like structures in non-endocrine cells. Biochem J, 2009. 418 (1): p. 81-91.

47. Hendy, G. N., et al., Targeted ablation of the chromogranin a (Chga) gene: normal neuroendocrine dense-core secretory granules and increased expression of other granins. Mol Endocrinol, 2006. 20 (8): p. 1935-47.

48. Fox, J. W., et al., Recombinant nidogen consists of three globular domains and mediates binding of laminin to collagen type IV. EMBO J, 1991. 10 (11): p. 3137-46.

49. Ho, M. S., et al., Nidogens-Extracellular matrix linker molecules. Microsc Res Tech, 2008. 71(5): p. 387-95.

50. McKee, K. K., et al., Role of laminin terminal globular domains in basement membrane assembly. J Biol Chem, 2007. 282 (29): p. 21437-47.

51. Poschl, E., et al., Collagen IV is essential for basement membrane stability but dispensable for initiation of its assembly during early development. Development, 2004. 131 (7): p. 1619-28.

52. Mayer, U., et al., Sites of nidogen cleavage by proteases involved in tissue homeostasis and remodelling. Eur J Biochem, 1993. 217 (3): p. 877-84.

53. Evseenko, D., et al., Identification of the critical extracellular matrix proteins that promote human embryonic stem cell assembly. Stem Cells Dev, 2009. 18 (6): p. 919-28.

54. Zhou, Y., et al., NID1, a new regulator of EMT required for metastasis and chemoresistance of ovarian cancer cells. Oncotarget, 2017. 8 (20): p. 33110-33121.

55. Robertson, N. G., et al., Subcellular localisation, secretion, and post-translational processing of normal cochlin, and of mutants causing the sensorineural deafness and vestibular disorder, DFNA9. J Med Genet, 2003. 40 (7): p. 479-86.

56. Nagy, I., M. Trexler, and L. Patthy, The second von Willebrand type A domain of cochlin has high affinity for type I, type II and type IV collagens. FEBS Lett, 2008. 582 (29): p. 4003-7.

57. Zhang, J., et al., BMP induces cochlin expression to facilitate self-renewal and suppress neural differentiation of mouse embryonic stem cells. J Biol Chem, 2013. 288 (12): p. 8053-60.

58. Tesar, P. J., et al., New cell lines from mouse epiblast share defining features with human embryonic stem cells. Nature, 2007. 448 (7150): p. 196-9.

59. Kunath, T., et al., FGF stimulation of the Erk1/2 signalling cascade triggers transition of pluripotent embryonic stem cells from self-renewal to lineage commitment. Development, 2007. 134 (16): p. 2895-902.

60. Ying, Q. L., et al., BMP induction of Id proteins suppresses differentiation and sustains embryonic stem cell self-renewal in collaboration with STAT3. Cell, 2003. 115 (3): p. 281-92.

61. Roberts, R. M., et al., Specification of trophoblast from embryonic stem cells exposed to BMP4. Biol Reprod, 2018. 99 (1): p. 212-224.

62. Oldershaw, R. A., et al., Directed differentiation of human embryonic stem cells toward chondrocytes. Nat Biotechnol, 2010. 28 (11): p. 1187-94.

63. Kurek, D., et al., Endogenous WNT signals mediate BMP-induced and spontaneous differentiation of epiblast stem cells and human embryonic stem cells. Stem Cell Reports, 2015. 4 (1): p. 114-28.

64. Loh, K. M., et al., Mapping the Pairwise Choices Leading from Pluripotency to Human Bone, Heart, and Other Mesoderm Cell Types. Cell, 2016. 166 (2): p. 451-467.

65. von Roemeling, C. A., et al., Neuronal pentraxin 2 supports clear cell renal cell carcinoma by activating the AMPA-selective glutamate receptor-4. Cancer Res, 2014. 74 (17): p. 4796-810.

66. Petersen, T. N., et al., SignalP 4.0: discriminating signal peptides from transmembrane regions. Nat Methods, 2011. 8 (10): p. 785-6.

67. Bendtsen, J. D., et al., Feature-based prediction of non-classical and leaderless protein secretion. Protein Eng Des Sel, 2004. 17 (4): p. 349-56.

68. Huang da, W., B. T. Sherman, and R. A. Lempicki, Bioinformatics enrichment tools: paths toward the comprehensive functional analysis of large gene lists. Nucleic Acids Res, 2009. 37 (1): p. 1-13.

69. Shannon, P., et al., Cytoscape: a software environment for integrated models of biomolecular interaction networks. Genome Res, 2003. 13 (11): p. 2498-504.

1. A method for determining the pluripotent state of a stem cell culture said method comprising culturing stem cells and
a) measuring the relative abundance in the culture media of two proteins selected from: Argininosuccinate synthetase 1 (ASS1), Creatine kinase B-type (CKB), chondromodulin-1 (CNMD), Cochlin (COCH), Fibroblast Growth Factor Receptor 1 (FGFR1), Follistatin (FST), Olfactomedin-like 3 (OLFML3), Serpin B1 (SERPINB1), Stomatin (STOM), A disintegrin and metalloproteinase with thrombospondin motifs 8 (ADAMTS8), Amyloid Beta Precursor Protein (APP), UDP-G1cNAc:BetaGal Beta-1,3-N-Acetylglucosaminyltransferase 7 (B3GNT7), Chromogranin A (CHGA), EGF-containing fibulin-like extracellular matrix protein 1 (EFEMP1), Ephrin type-A receptor 1 (EPHA1), Exostosin-like 2 (EXTL2), Fibulin 2 (FBLN2), Fibroblast Growth Factor 2 (FGF2), Plasma alpha-L-fucosidase (FUCA2), Polypeptide N-acetylgalactosaminyltransferase 1 (GALNT), Gamma-Glutamyl Hydrolase (GGH), Beta-hexosaminidase subunit beta (HEXB), Insulin-like growth factor-binding protein 4 (IGFBP4), Nidogen 1 (NID1), Neuronal Pentraxin-2 (NPTX2), Neurotensin (NTS), Protocadherin beta-5 (PCDHBS), Semaphorin 3A (SEMA3A), Semaphorin 3F (SEMA3F), Secreted Frizzled Related Protein 2 (SFRP2), Metalloproteinase inhibitor 4 (TIMP4), Tumor necrosis factor receptor superfamily member 8 (TNFRSF8) and WAP four-disulfide core domain protein 2 (WFDC2) and comparing said relative abundance to a reference value; and/or
b) measuring the absolute abundance of one or more protein in the culture media selected from Argininosuccinate synthetase 1 (ASS1), Creatine kinase B-type (CKB), chondromodulin-1 (CNMD), Cochlin (COCH), Fibroblast Growth Factor Receptor 1 (FGFR1), Follistatin (FST), Olfactomedin-like 3 (OLFML3), Serpin B1 (SERPINB1), Stomatin (STOM), A disintegrin and metalloproteinase with thrombospondin motifs 8 (ADAMTS8), Amyloid Beta Precursor Protein (APP), UDP- GlcNAc:BetaGal Beta-1,3-N-Acetylglucosaminyltransferase 7 (B3GNT7), Chromogranin A (CHGA), EGF-containing fibulin-like extracellular matrix protein 1 (EFEMP1), Ephrin type-A receptor 1 (EPHA1), Exostosin-like 2 (EXTL2), Fibulin 2 (FBLN2), Fibroblast Growth Factor 2 (FGF2), Plasma alpha-L-fucosidase (FUCA2), Polypeptide N-acetylgalactosaminyltransferase 1 (GALNT), Gamma-Glutamyl Hydrolase (GGH), Beta-hexosaminidase subunit beta (HEXB), Insulin-like growth factor binding protein 4 (IGFBP4), Nidogen 1 (NID1), Neuronal Pentraxin-2 (NPTX2), Neurotensin (NTS), Protocadherin beta-5 (PCDHB5), Semaphorin 3A (SEMA3A), Semaphorin 3F (SEMA3F), Secreted Frizzled Related Protein 2 (SFRP2), Metalloproteinase inhibitor 4 (TIMP4), Tumor necrosis factor receptor superfamily member 8 (TNFRSF8) and WAP four-disulfide core domain protein 2 (WFDC2) and comparing said absolute abundance to a reference value.

2. The method of claim 1 wherein the relative abundance of one or more of the following protein pairs is measured
a) COCH paired with CHGA, NID1, NPTX2, SEMA3A; FGFR1 paired with CHGA, NID1, NPTX2, SEMA3A; FST paired with CHGA, NID1, NPTX2, SEMA3A; and/or OLFML3 CHGA, NID1, NPTX2, SEMA3A CHGA, NID1, NPTX2, SEMA3A;
b) ADAMTS8 paired with ASS1, CKB, CNMD, COCH, FGFR1, FST, OLFML3, SERPINB9 or STOM;
c) APP paired with ASS1, CKB, CNMD, COCH, FGFR1, FST, OLFML3, SERPINB9 or STOM;
d) B3GNT7 paired with ASS1, CKB, CNMD, COCH, FGFR1, FST, OLFML3, SERPINB9 or STOM;
e) CHGA paired with ASS1, CKB, CNMD, COCH, FGFR1, FST, OLFML3, SERPINB9 or STOM;
f) EFEMP1 paired with ASS1, CKB, CNMD, COCH, FGFR1, FST, OLFML3, SERPINB9 or STOM;
g) EPHA1 paired with ASS1, CKB, CNMD, COCH, FGFR1, FST, OLFML3, SERPINB9 or STOM;
h) EXTL2 paired with ASS1, CKB, CNMD, COCH, FGFR1, FST, OLFML3, SERPINB9 or STOM;
i) FBLN2 paired with ASS1, CKB, CNMD, COCH, FGFR1, FST, OLFML3, SERPINB9 or STOM;
j) FGF2 paired with ASS1, CKB, CNMD, COCH, FGFR1, FST, OLFML3, SERPINB9 or STOM;
k) FUCA2 paired with ASS1, CKB, CNMD, COCH, FGFR1, FST, OLFML3, SERPINB9 or STOM;
l) GALNT1 paired with ASS1, CKB, CNMD, COCH, FGFR1, FST, OLFML3, SERPINB9 or STOM;
m) GGH paired with ASS1, CKB, CNMD, COCH, FGFR1, FST, OLFML3, SERPINB9 or STOM;
n) HEXB paired with ASS1, CKB, CNMD, COCH, FGFR1, FST, OLFML3, SERPINB9 or STOM;
o) IGFBP4 paired with ASS1, CKB, CNMD, COCH, FGFR1, FST, OLFML3, SERPINB9 or STOM;
p) NID1 paired with ASS1, CKB, CNMD, COCH, FGFR1, FST, OLFML3, SERPINB9 or STOM;
q) NPTX2 paired with ASS1, CKB, CNMD, COCH, FGFR1, FST, OLFML3, SERPINB9 or STOM;
r) NTS paired with ASS1, CKB, CNMD, COCH, FGFR1 , FST, OLFML3, SERPINB9 or STOM;
s) PCDHB5 paired with ASS1, CKB, CNMD, COCH, FGFR1, FST, OLFML3, SERPINB9 or STOM;
t) SEMA3A paired with ASS1, CKB, CNMD, COCH, FGFR, FST, OLFML3, SERPINB9 or STOM;
u) SEMA3F paired with ASS1, CKB, CNMD, COCH, FGFR, FST, OLFML3, SERPINB9 or STOM;
v) SFRP2 paired with ASS1, CKB, CNMD, COCH, FGFR, FST, OLFML3, SERPINB9 or STOM;
w) TIMP4 paired with ASS1, CKB, CNMD, COCH, FGFR1, FST, OLFML3, SERPINB9 or STOM;
x) TNFRSF8 paired with ASS1, CKB, CNMD, COCH, FGFR1, FST, OLFML3, SERPINB9 or STOM; and/or
y) WFDC2 paired with ASS1, CKB, CNMD, COCH, FGFR1, FST, OLFML3, SERPINB9 or STOM.

3. The method of claim 1 comprising measuring the absolute abundance of one or more protein selected from COCH, FGFR1, FST, OLFML3, CHGA, NID1, NPTX2, SEMA3 A.

4. The method of claim 1 comprising measuring the relative abundance of one or more protein pair selected from COCH paired with CHGA, NID1, NPTX2, SEMA3A; FGFR1 paired with CHGA, NID1, NPTX2, SEMA3A; FST paired with CHGA, NID1, NPTX2, SEMA3A; and/or OLFML3 paired with CHGA, NID1, NPTX2, SEMA3A.

5. The method of claim 1 wherein the relative abundance of two proteins is compared to a reference value and wherein said reference value is the relative abundance of the two proteins in a pluripotent stem cell (PSCs) culture.

6. The method of claim 1 wherein the absolute abundance of two proteins is compared to a reference value and wherein said reference value is the absolute abundance of the two proteins in a pluripotent stem cell (PSCs) culture.

7. The method of claim 1 further comprising correlating said relative or absolute abundance measured with the presence of undifferentiated cells in the culture.

8. The method of claim 1, wherein an elevated or reduced individual abundance of the one or more proteins and/or relative abundance the one or more protein pair compared to the reference value of indicates the loss of pluripotency or potential loss of pluripotency.

9. The method of a prcccding claim 1, wherein the method comprises determining a reference individual abundance and/or relative abundance.

10. (canceled)

11. The method of claim 1, wherein the PSCs are iPSCs.

12.-13. (canceled)

14. The method as claimed in claim 1, wherein the culture media is assessed:
a) at a regular time interval, e.g. 12, 24, 48 or 72 hours, after being seeded with pluripotent stem cells; or
b) on a continuous basis.

15. (canceled)

16. The method as claimed in claim 1, wherein the absolute or relative abundance of the protein is assessed by Mass Spectrometry (MS).

17. The method as claimed in claim 1, wherein the absolute or relative abundance of the protein is assessed by one or more antibody or antibody fragment, such as by an Enzyme-Linked ImmunoSorbent Assay (ELISA) and/or similar technique.

18. (canceled)

19. The method as claimed in claim 1, for detecting the loss of pluripotency or potential loss of pluripotency and/or recovery of stable pluripotent state.

20.-23. (canceled)

24. A kit comprising:
a) one or more components which can bind to or otherwise detect one or more of the following proteins: ASS1, CKB, CNMD, COCH, FGFR1, FST, OLFML3, SERPINB1, STOM, ADAMTS8, APP, B3GNT7, CHGA, EFEMP1, EPHA1, EXTL2, FBLN2, FGF2, FUCA2, GALNT, GGH, HEXB, IGFBP4, NID1, NPTX2, NTS, PCDHB5, SEMA3A, SEMA3F, SFRP2, TIMP4, TNFRSF8 and/or WFDC2; and optionally
b) means for quantifying binding and/or detection of the one or more components with the one or more proteins, such as an antibody or antibody fragment.

25. The kit of claim 24, wherein the kit comprises a control representing the quantitative value of the proteins and/or protein abundance expected when cells are in a pluripotent state, such as wherein the control allows a user to establish baseline values of pluripotent cells in their own culture system to increase the applicability and accuracy of the method.

26. (canceled)

27. The kit of claim 24, wherein the means for detecting quantitative binding of the one or more components with the one or more proteins:
a) further indicates whether the one or more proteins are elevated or reduced in a sample; and/or
b) indicates the relative quantity of each of the two or more separate proteins so as to enable the individual and/or relative abundance of the two or more proteins to be calculated.

28. The kit of claim 24, wherein the kit comprises:
a) two or more components which can bind to two or more separate proteins and the means for detecting quantitative binding detects the quantitative binding of the two or more components with the two or more separate proteins;
b) a first component which can bind to a first protein selected from: ASS1, CKB, CNMD, COCH, FGFR1, FST, OLFML3, SERPINB1, STOM; and a second component which can bind to a second protein selected from: ADAMTS8, APP, B3GNT7, CHGA, EFEMP1, EPHA1, EXTL2, FBLN2, FGF2, FUCA2, GALNT, GGH, HEXB, IGFBP4, NID1, NPTX2, NTS, PCDHB5, SEMA3A, SEMA3F, SFRP2, TIMP4, TNFRSF8 and/or WFDC2); and/or
c) one or more reaction vessels so as to allow components to be mixed with spent or conditioned growth medium (or fractions thereof).

29.-32. (canceled)

33. The kit as claimed in claim 24, wherein the kit comprises:
a) an Enzyme-Linked ImmunoSorbent Assay (ELISA) and/or similar immune assay; and/or
b) components suitable for use in detecting proteins using Mass Spectrometry.

34. (canceled)

35. The kit of claim 24, for assessing the pluripotent state of pluripotent stem cells in the spent or conditioned growth media of the cells.
